(19) United States
(12) Patent Application Publication
(10) Pub. No.: US 2021/0283597 A1
(43) Pub. Date: Sep. 16, 2021

(54) POLYMER-BASED BIOSPECIMEN COLLECTION DEVICES AND USES THEREOF

(71) Applicant: VAXESS TECHNOLOGIES, INC., Cambridge, MA (US)

(72) Inventors: Jonathan A. Kluge, Cambridge, MA (US); Alexander Beliveau, Cambridge, MA (US); Adrian Benton Li, Cambridge, MA (US); Matthew Dirckx, Cambridge, MA (US); Michael A. Schrader, Cambridge, MA (US); Kathryn M. Kosuda, Cambridge, MA (US); Elizabeth Whitney Johansen, Cambridge, MA (US)

(73) Assignee: VAXESS TECHNOLOGIES, INC., Cambridge, MA (US)

(21) Appl. No.: 17/157,451

(22) Filed: Jan. 25, 2021

Related U.S. Application Data
(63) Continuation of application No. PCT/US2019/043727, filed on Jul. 26, 2019.
(60) Provisional application No. 62/845,537, filed on May 9, 2019.
(60) Provisional application No. 62/711,269, filed on Jul. 27, 2018.

(51) Int. Cl. B01L 3/00; G01N 1/38

(52) U.S. Cl. CPC B01L 3/5023; G01N 1/38; B01L 2300/046; B01L 2300/0838; B01L 2300/069

(57) ABSTRACT

The present invention generally relates to the collection, storage, and stabilization of biological samples (e.g., bodily fluids, such as whole blood, blood serum, and blood plasma), and analytes thereof. More particularly, the present invention relates to polymer-based biological sample collection devices, and methods of making and using the same.

### GOVERNMENT SUPPORT

This invention was made with support from the federal government under Grant Nos. 2R44ES025516-02 and 1R43ES025516-01 awarded by the National Institute of Environmental Health Sciences. The U.S. government has certain rights in the invention.

### FIELD OF THE INVENTION

The present invention generally relates to the collection, storage, and stabilization of biological samples (e.g., bodily fluids, such as whole blood, blood serum, blood plasma, urine, and/or saliva), and analytes thereof. More particularly, the present invention relates to biological sample collection devices (e.g., polymer-based biological collection devices), stabilizing polymer compositions (e.g., substantially dried polymer compositions), and methods of making and using the same.

### BACKGROUND OF THE DISCLOSURE

Biological sample analysis remains a mainstay of human health and disease screening, diagnosis, and health optimization, and monitoring. Biological sample degradation, which can occur during sample storage and/or transport, can impact the effectiveness of downstream laboratory assays and instrumentation in the analysis of biological samples, e.g., to detect and/or measure analytes of interest. Many analytes that are useful in human disease screening, diagnosis, and therapeutic monitoring are known to be labile and/or sensitive to changes in environmental conditions, e.g., changes in temperature, light, and/or humidity. Use of the cold chain to stabilize biological samples during transportation and/or storage is not always feasible, e.g., due to energy requirements.

Accordingly, there remains a strong need for new methods and devices for the stabilization and storage of biological samples to reduce the incidence and/or effects of sample degradation, e.g., from changing environmental conditions. Further, there exists a need for new methods and devices that enable the transport and/or storage of biological samples at ambient conditions, and without the need for the cold chain.

### SUMMARY OF THE INVENTION

The present invention provides, at least in part, stabilizing polymer compositions (e.g., silk fibroin compositions) and biological sample collection, separation, and recovery and/or processing devices that can be configured to stabilize a biological sample or an analyte thereof, e.g., prior to downstream analysis by laboratory assays and/or instrumentation known in the art.

The devices and compositions described herein are configured to enable storage and/or transport of a biological sample, e.g., at ambient conditions without significant sample degradation and/or without the need for the cold chain. In some embodiments, the devices described herein are configured to enable the formation of a substantially dried polymer composition (e.g., a substantially dried silk fibroin composition) comprising a biological sample (e.g., or at least a fraction, a component, and/or an analyte of a biological sample).

The devices and stabilizing polymer compositions (e.g., silk fibroin compositions) described herein can confer increased stability to a biological sample (e.g., or at least a fraction, a component, and/or an analyte of a biological sample), e.g., as compared to a biological sample collected in a device in the absence of a stabilizing polymer composition (e.g., a silk fibroin composition) described herein. In some instances, the terms “stabilizing polymer compositions,” “polymer composition,” and “silk fibroin composition” are used interchangeably herein.

In some embodiments, the devices and stabilizing polymer compositions (e.g., silk fibroin compositions) described herein are configured to improve the stability of a biological sample, e.g., by reducing the degradation, misfolding, denaturation, aggregation, and/or inactivation of an analyte of the biological sample, e.g., over a period of time at ambient temperatures. Use of these devices and polymer compositions can improve downstream sample analysis, e.g., after prolonged storage and/or transport over multiple days, weeks, months, and/or years, by preserving the structure, integrity, configuration, function, and/or activity of the biological sample or an analyte thereof over the time period of storage and/or transport.

Further, the devices and stabilizing polymer compositions (e.g., silk fibroin compositions) described herein can be configured to be compatible with and to enable the analysis (e.g., recovery) of an analyte from a biological sample using laboratory assays and/or instrumentation known in the art. Accordingly, polymer-based (e.g., silk fibroin-based) biological sample collection, separation, and recovery and/or processing devices, as well as methods of making and using the same, are disclosed.

In one aspect, the invention features a biological sample collection and processing device comprising: (i) a sample collection and mixing portion configured to receive and mix a biological sample, the sample collection and mixing portion including a stabilizing polymer compositions (e.g., silk fibroin compositions) as described herein, the sample collection and mixing portion being configured to allow a precise and sufficient amount of the biological sample and the silk fibroin composition to form a mixture; and (ii) a drying and recovery portion configured to allow substantially drying the biological sample and/or the mixture, optionally, wherein the drying is by air-drying and/or desiccant facilitated air-drying.

In some embodiments, the device comprises about 0.01 mg to about 100 mg (e.g., about 0.05 mg to about 80 mg, about 0.75 mg to about 50 mg, about 0.75 mg to about 6 mg, about 1 mg to about 50 mg, about 1 mg to about 25 mg, about 1 mg to about 10 mg, about 2 to about 10 mg, about 3 to about 6 mg, or about 0.01 mg to about 10 mg) of a stabilizing polymer composition (e.g., a silk fibroin composition). In some embodiments, the stabilizing polymer composition (e.g., silk fibroin composition) is substantially dried (e.g., lyophilized, air-dried, or spray dried), or a liquid and disposed at a location of the device. In some embodiments, the stabilizing polymer composition is disposed in a sample collection and mixing portion, e.g., a capillary tube, of a device described herein.

In some embodiments, the stabilizing polymer composition (e.g., silk fibroin composition) comprises about 0.1% w/v to about 20% w/v polymer (weight of the polymer with respect to the volume of biological sample) (e.g., about 0.5% w/v to about 15% w/v, about 0.75% w/v to about 10% w/v, about 1% w/v to about 5% w/v, about 1% w/v to about 3% w/v, about 1% w/v to about 2% w/v, or about 5% w/v to about 10% w/v polymer).

In some embodiments, when the polymer composition comprises a silk fibroin composition, and the silk fibroin composition can comprises about 0.1% w/v to about 20% w/v silk fibroin (weight of the silk fibroin with respect to the volume of sample) (e.g., about 0.5% w/v to about 15% w/v, about 0.75% w/v to about 10% w/v, about 1% w/v to about 5% w/v, about 1% w/v to about 3% w/v, about 1% w/v to about 2% w/v, or about 5% w/v to about 10% w/v silk fibroin). The silk fibroin composition can comprise a population of silk fibroin having an average molecular weight of about 1 kDa to about 250 kDa (e.g., about 1 kDa to about 20 kDa, about 20 kDa to about 40 kDa, about 40 kDa to about 60 kDa, about 60 kDa to about 80 kDa, about 80 kDa to about 100 kDa, about 100 kDa to about 120 kDa, about 120 kDa to about 140 kDa, about 140 kDa to about 160 kDa, about 160 kDa to about 180 kDa, about 180 kDa to about 200 kDa). In some embodiments, the silk fibroin composition comprises: (i) a 10 minute boil (10 MB) silk fibroin composition, (ii) a 30 minute boil (30 MB) silk fibroin composition, (iii) a 60 minute boil (60 MB) silk fibroin composition, (iv) a 120 minute boil (120 MB) silk fibroin composition, (v) a 180 minute boil (180 MB) silk fibroin composition, (vi) a 240 minute boil (240 MB) silk fibroin composition, (vii) a 300 minute boil (300 MB) silk fibroin composition, (vii) a 360 minute boil (360 MB) silk fibroin composition, (ix) a 420 minute boil (420 MB) silk fibroin composition, and/or (x) a 480 minute boil (480 MB) silk fibroin composition.

In some embodiments, the stabilizing polymer composition (e.g., silk fibroin composition) can further include an excipient. The excipient may be chosen from, e.g., a sugar and/or a sugar alcohol (e.g., sucrose, trehalose, maltose, sorbitol, mannitol, glycerol, or a combination thereof), a protein (e.g, a silk fibroin protein, a gelatin protein, an albumin protein, an elastin protein, a collagen protein, a heparin protein, and/or a cellulose protein), a polymer (e.g., polyethylene glycol (PEG), polyethylene oxide (PEO), hyaluronic acid, carboxymethylcellulose (CMC), polyvinylpyrrolidone (PVP), polyvinyl alcohol (PVA)), a divalent cation (e.g., Ca²⁺, Mg²⁺, Mn²⁺, and Cu²⁺), a salt (e.g., monosodium glutamate), a buffer (e.g., a Tris buffer, a Tris EDTA buffer, a citrate buffer, a phosphate buffer, a MOPS buffer, and/or a HEPES buffer), an amino acid (e.g., L-glutamic acid and/or lysine), and solvents (e.g., DMSO, methanol, and/or ethanol). In some embodiments, the excipient is a sugar (e.g., sucrose). In some embodiments, the excipient is a buffer (e.g., HEPES).

In some embodiments, the excipient may be present in an amount between about 0.01 mg to about 100 mg excipient (e.g., about 0.05 mg to about 80 mg, about 0.75 mg to about 50 mg, about 0.75 mg to about 6 mg, about 1 mg to about 50 mg, about 1 mg to about 25 mg, about 1 mg to about 10 mg, about 2 to about 10 mg, about 3 to about 6 mg, or about 0.01 mg to about 10 mg).

In some embodiments, the excipient may be present in an amount between about 0.1% w/v to about 20% w/v excipient (weight of the excipient with respect to the volume of biological sample) (e.g., about 0.5% w/v to about 15% w/v, about 0.75% w/v to about 10% w/v, about 1% w/v to about 5% w/v, about 1% w/v to about 3% w/v, about 1% w/v to about 2% w/v, about 5% w/v to about 10% w/v), e.g., before drying.

In some embodiments, the w/v amount of an excipient described herein is with respect to the biological sample, the total liquid formulation, or the polymer (e.g., silk fibroin)).

In some embodiments, the sample collection and mixing portion includes a tube, e.g., a capillary tube. In some embodiments, the drying and recovery portion is a container, e.g., a removable container, configured to be releasably secured to the sample collection and mixing portion. In some embodiments, the sample and mixing portion and the drying and recovery portion include a common structure, e.g., a capillary tube.

In some embodiments, the device is configured to reconstitute a dried polymer composition (e.g., silk fibroin composition) to a pre-defined volume with a biological sample. In some embodiments, the collection and mixing portion and the drying and recovery portion (e.g., the capillary tube) is configured to meter the biological sample. In some embodiments, the collection and mixing portion and the drying and recovery portion (e.g., the capillary tube) is configured to meter about 10 μL, to about 300 μL, (e.g., about 10 μL, about 20 μL, about 30 μL, about 40 μL, about 50 μL, about 60 μL, about 70 μL, about 80 μL, about 90 μL, about 100 μL, about 110 μL, about 120 μL, about 130 μL, about 140 μL, about 150 μL, about 160 μL, about 170 μL, about 180 μL, about 190 μL, about 200 μL, about 210 μL, about 220 μL, about 230 μL, about 240 μL, about 250 μL, about 260 μL, about 270 μL, about 280 μL, about 290 μL, or about 300 μL) of the biological sample.

In some embodiments, the biological sample collection and recovery and/or processing device further comprising a porous membrane positioned in the collection and mixing portion. In some embodiments, the biological sample collection and recovery device further comprises a sample collector positioned in the collection and mixing portion, the sample collector being coated with anticoagulant (e.g., EDTA, heparin, sodium citrate, acid-citrate-dextrose, and oxalate). In some embodiments, the sample collector includes a finger wiper feature to facilitate transferring biological sample from finger into collector. In some embodiments, the collection and mixing portion includes a removable absorbent cap configured to absorb excess sample. In some embodiments, the collection and mixing portion includes a removable desiccant cap configured to include desiccant material.

In some embodiments, the drying and recovery portion includes a tube and a well positioned in the tube, the well being configured to contain the biological sample, the polymer composition (e.g., silk fibroin composition), and/or the mixture (e.g., the mixture for drying) for easy elution, e.g., of a dried and/or partially dried composition as described herein.

In some embodiments, the biological sample collection and recovery further comprises a plunger or material configured to bend to create a negative pressure in the well thereby drawing the sample through a porous membrane and into the well.

In some embodiments, the tube with sample well contains a desiccant, e.g., a desiccant configured to facilitate air-drying of the biological sample and/or the mixture. In some embodiments, the desiccant is chosen from the group comprising a molecular sieve, a silica gel, a montmorillonite clay, a calcium oxide, a calcium sulfate, and mixtures thereof. In some embodiments, the well includes a geometry to spread the collected sample over a large surface area. In some embodiments, the well includes a centrally located dimple and ribs to provide adequate sample depth for a pipette and guide the tip of a pipette to the dimple to enable a pipette to access sample. In some embodiments, the well includes posts to create a meniscus creating a larger surface area to facilitate faster sample drying. In some embodiments, the well is sufficiently narrow and deep to provide adequate sample depth to receive a tip of a pipette and can be tilted to spread sample over large side surface for efficient drying.

In one aspect, the invention features a sample collection device comprising: a tube; a well positioned in the tube, the well being configured to receive and dry a mixture of a biological sample and a silk fibroin composition; and a sample collection assembly configured to receive and mix the biological sample with the silk fibroin composition, and to deliver the mixture to the well, the sample collection assembly being configured to allow a sufficient amount of the biological sample and the silk fibroin composition to form the mixture.

In some embodiments, the sample collection assembly includes an absorbent cap that contains an absorbent material, capillary, or capillary array; a sample collector that is coated with anticoagulant; a plasma separation membrane; and a capillary that includes the silk fibroin composition and is coated with anticoagulant. In some embodiments, the sample collection assembly further comprises a capillary and plunger holder configured to facilitate the mixing of the biological sample and the silk fibroin composition. In some embodiments, the sample collection assembly includes a plunger configured to create a negative pressure in the well when rotating the capillary and plunger holder on a thread, pulling the plunger until the capillary and plunger holder hits a thread stop or engages a detent (e.g., a catch). In some embodiments, when rotating the capillary and plunger holder, the biological sample enters the capillary with lyophilized silk formulation and anticoagulant and exits into the well. In some embodiments, the thread stop is configured to be removed to enable the sample collection assembly to be unscrewed and removed from the well. In some embodiments, after removing the sample collection assembly, the tube is capped with a desiccant cap having a desiccant to assist in drying the biological sample and silk mixture in the well.

In one aspect, the invention features a method of collecting, mixing and storing a sample with a device described herein. In some embodiments, the device is configured to be shipped.

In one aspect, the invention features a biological sample collection and recovery device (e.g., a polymer-based device biological sample collection and recovery device), comprising (i) a sample collection portion configured to receive a biological sample; (ii) a mixing portion including a polymer composition as described herein, e.g., a silk fibroin composition, the mixing portion being configured to allow a sufficient amount of the biological sample and the polymer composition, e.g., the silk fibroin composition, to form a mixture (e.g., a mixture for stabilization by drying); and (iii) a drying and recovery portion configured to allow substantially drying the biological sample and/or the mixture, optionally, wherein the drying is by air-drying and/or by desiccant facilitated air-drying. In some embodiments, the drying and recovery portion can be configured to enable reconstitution of a substantially dried polymer composition for downstream analysis, e.g., by laboratory assays and/or instrumentation further described herein. In some embodiments, the recovery portion can be optionally detachable from the device. In some embodiments, the recovery portion is configured to be compatible with laboratory assays and/or instrumentation known in the art.

In some embodiments, the mixing portion is a tube, e.g., a capillary tube. The mixing portion can include a removable base, e.g., having a pair of snap fit members configured to be received within openings formed in the sample collection portion. In some embodiments, the mixing portion and the drying and recovery portion include a common structure, e.g., a capillary tube or a base.

In some embodiments, the drying and recovery portion can be a base, e.g., a removable base, configured to be releasably secured to the sample collection portion. The drying portion may also be configured to be a recovery portion. The device can further comprise a porous membrane, e.g., positioned in the removable base. In some embodiments, the drying and recovery portion, e.g., the removable base, includes a well configured to contain the biological sample, the polymer composition as described herein, and/or the mixture for easy elution, e.g., of a dried and/or partially dried biological sample or polymer composition as described herein. In some embodiments, the porous membrane is seated on the well.

Without wishing to be bound by theory, easy reconstitution of the dried and/or partially dried biological sample (e.g., a substantially dried polymer composition) enables the generation of a reaction mixture and/or an analysis sample that may be analyzed using laboratory assays and/or instrumentation well known in the art and described further herein.

In some embodiments, the sample collection portion includes a funnel-shaped collector having a scoop to aid in wiping biological sample into the collector. The collector may be configured to direct the biological sample to an opening that contains the mixing portion, e.g., a capillary tube, and wherein the mixing portion is further configured to direct the biological sample through the container, e.g., to the drying and recovery portion.

In some embodiments, the base contains a desiccant, and optionally wherein the desiccant is chosen from the group comprising a molecular sieve, a silica gel, a montmorillonite clay, a calcium oxide, a calcium sulfate, and mixtures thereof. The desiccant can be included in the device in a sufficient amount to facilitate air-drying (e.g., also referred to herein as desiccant facilitated air drying) of the biological sample and/or mixture, e.g., to produce a stabilized, substantially dried polymer composition as described herein.

In some embodiments, the sample collection portion and the mixing portion, e.g., the removable base, are fabricated from, e.g., a molded plastic, such as polypropylene, polystyrene, polyethylene terephthalate (PET), polydimethylsiloxane (PDMS), poly(methyl methacrylate) (PMMA), poly(tetrafluoroethylene) (PTFE), and/or from a paper.

In some embodiments, the device may further comprise a sample separation accessory configured to separate a fraction, a component, and/or an analyte of the biological sample (e.g., to separate plasma from a whole blood sample). The sample separation accessory can include, e.g., a disc-shaped top portion and a disc-shaped base portion, which can be configured to be releasably secured to one another. In some embodiments, the sample separation accessory is configured to be retrofitted into an end of a sample mixing portion, e.g., a capillary tube, of the collection device. In some embodiments, the disc-shaped base portion includes a well configured to receive a biological sample, e.g., a whole blood sample, separation membrane and a collection layer configured to passively receive a fraction, a component and/or an analyte of the biological sample, e.g., plasma and/or serum. The disc-shaped top portion and the disc-shaped base portion can be configured with a locking mechanism to releasably secure the disc-shaped top portion to the disc-shaped base portion. The locking mechanism can include a bayonet connector having cylindrical male side with one or more radial pins provided on the disc-shaped top portion, and a female receptor with matching L-shaped slot(s) provided on the disc-shaped bottom portion to keep the two parts locked together when assembled.

In some embodiments, each slot is L-shaped to receive the pin as the pin slides into a vertical arm of the L-shaped slot and is moved across the horizontal arm of the L-shaped slot.

In one aspect, the invention features a biological sample collection and recovery device comprising: (i) a sample collection portion configured to receive a biological sample; (ii) a mixing portion including a polymer composition as described herein, e.g., a silk fibroin composition, the mixing portion being configured to allow a sufficient amount of the biological sample and the polymer composition to form a mixture; (iii) a drying and recovery portion, e.g., a base portion, configured to be secured to the sample collection portion and further configured to allow substantially drying the biological sample and/or the mixture, optionally, wherein the drying is by air-drying and/or by desiccant facilitated air-drying; and (iv) a porous membrane provided in the base portion, the porous membrane being configured to enable airflow and prohibit fluid flow therethrough.

In some embodiments, the sample collection portion includes a top part and a bottom part that is disposed under the top part and is configured to support and position the sample mixing portion, e.g., a capillary tube. The top part of the sample collection portion can be configured to include a window to enable a user to monitor when collection of a sample is complete. The top part and the bottom part can optionally be assembled together to create a channel to hold the capillary tube. The capillary tube can be configured to include a polymer composition, e.g., a silk-fibroin composition as described herein, and/or an anticoagulant, e.g., EDTA, heparin, and/or sodium citrate, optionally wherein the anticoagulant is applied as a coating and/or as a layer.

The top part of the sample collection portion can include a funnel-shaped collector having a scoop to aid in biological sample collection. In some embodiments, the collector is configured to direct a sample to an opening that is in fluid communication with the mixing portion, e.g., a capillary tube.

In some embodiments, the device can further comprise a piston or plunger provided to pressurize fluid within the mixing portion, e.g., the capillary tube, to enhance the flow of the biological sample to the well. The drying and recovery portion, e.g., the base portion, optionally includes a well configured to contain the biological sample for easy elution. In some embodiments, the base portion contains a desiccant in part of the drying and recovery portion, e.g., the base portion, next to the well. The desiccant can be chosen from the group comprising a molecular sieve, a silica gel, a montmorillonite clay, a calcium oxide, a calcium sulfate, and mixtures thereof.

In one aspect, the invention features a plasma separation card comprising: (i) a top layer portion; (ii) a mixing portion, e.g., a bottom layer portion, including a polymer composition as described herein, e.g., a silk fibroin composition, the mixing portion being configured to allow a sufficient amount of the biological sample and the polymer composition to form a mixture; (iii) a tape layer; and (iv) a drying and recovery portion, e.g., a lateral flow plasma separation membrane comprising a polymer composition as described herein, e.g., a silk fibroin composition, configured to allow substantially drying the biological sample and/or the mixture, optionally, wherein the drying is by air-drying and/or by desiccant facilitated air-drying.

In some embodiments, the top layer portion includes a funneled-shaped scoop inlet to direct a biological sample into the card. The top layer portion can further include a window formed therein to evaluate when card is fully loaded with a biological sample.

In some embodiments, the mixing portion, e.g., the bottom layer portion, includes a pathway and a collection area formed therein to assist the flow of the biological sample, e.g., blood, from the pathway to the collection area. The mixing portion, e.g., the bottom layer portion, can include a coating and or a surface treatment to modify (e.g., enhance) surface wetting (e.g., modify hydrophilicity, e.g., to direct biological sample flow throughout the device) and/or reduce clotting and/or modify protein binding. Exemplary surface treatments include, but are not limited to, a silane chemistry treatment, an application of a coating comprising a hydrophobic and/or a hydrophilic polymer, an application of an anticoagulant coating (e.g., a heparin coating, a Cit coating, and/or an EDTA coating), and/or an application of a coating comprising a surfactant. In some embodiments, the surface treatment comprises Silwet. In some embodiments, the surface treatment comprises a hydrophobic polymer, e.g., Tensimorph, Tenistat, TecophiliTPU, HydroThane, and/or ARFlow film. In some embodiments, the mixing portion, e.g., the bottom layer portion, contains a polymer composition as described herein, such as a lyophilized, powdered, and/or solid composition. The mixing portion, e.g., the bottom layer portion, can include grooves to aid in mixing, and can be press-fit or glued with the top layer to create a seal.

In some embodiments, the drying and recovery portion, e.g., the lateral flow plasma separation membrane, includes paper that can be removed from the card and/or punched within the card for analysis of sample, e.g., using standard laboratory assays and/or instrumentation.

In some embodiments, the collection device is enclosed in a transport vessel with sealed desiccant to enhance drying.

In one aspect, the invention features a sample separation accessory configured to separate a fraction, a component, and/or an analyte, e.g., plasma, from a biological sample, e.g., a whole blood sample, the accessory comprising: (i) a disc-shaped top portion; (ii) a disc-shaped base portion configured to be releasably secured to the disc-shaped top portion; and (iii) a locking mechanism to releasably secure the disc-shaped top portion to the disc-shaped base portion.

In some embodiments, the locking mechanism includes a bayonet connector having cylindrical male side with one or more radial pins provided on the disc-shaped top portion, and a female receptor with matching L-shaped slot(s) provided on the disc-shaped bottom portion to keep the two parts locked together when assembled.

In some embodiments, the accessory is configured to be retrofitted into an end of a mixing portion, e.g., a capillary tube, of a collection device.

In some embodiments, the disc-shaped base portion includes a well configured to receive the mixing portion, e.g., a plasma separation membrane optionally impregnated with a polymer composition as described herein, and the collection layer being configured to passively receive the fraction, the component and/or the analyte of the biological sample, e.g., the plasma.

In some embodiments, a polymer-based biological sample collection and/or separation devices described herein can be configured to enable the drying (e.g., air-drying) of a biological sample and/or a mixture (e.g., a polymer mixture for drying) as described herein, such that the drying can occur in the device (e.g., in a drying and recovery portion of the device) within about 48 hours (e.g., within about 1-5 minutes, e.g., within about 1-10 minutes, e.g., within about 1-20 minutes, e.g., within about 1-30 minutes, e.g., within about 1-40 minutes, e.g., within about 1-50 minutes, e.g., within about 1-60 minutes, e.g., within about 1, 2, 3, 4, 5, 6, 7, 8, 9, 10, 20, 30, 40, 50, or 60 minutes, e.g., within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours). In some embodiments, the drying (e.g., air-drying and/or desiccant facilitated air-drying) can occur within about 1 to about 10 hours (e.g., about 1, about 2, about 3, about 4 hours, about 5 hours, about 6 hours, about 7 hours, about 8 hours, about 9 hours, or about 10 hours). In some embodiments, the drying (e.g., air-drying and/or desiccant facilitated air-drying) can occur within about 2 to about 8 hours (e.g., about 4 hours, about 5 hours, or about 6 hours). In some embodiments, the drying (e.g., air-drying and/or desiccant facilitated air-drying) can occur within about 4 to about 6 hours (e.g., about 4 hours, about 5 hours, or about 6 hours). In some embodiments, the drying (e.g., air-drying and/or desiccant facilitated air-drying) can occur within about 6 hours. In some embodiments, the drying (e.g., air-drying and/or desiccant facilitated air-drying) can occur within about 5 hours. In some embodiments, the drying (e.g., air-drying and/or desiccant facilitated air-drying) can occur within about 4 hours. In some embodiments, the drying (e.g., air-drying and/or desiccant facilitated air-drying) can occur within about 3 hours. In some embodiments, the drying (e.g., air-drying and/or desiccant facilitated air-drying) can occur within about 2 hours. In some embodiments, the drying (e.g., air-drying and/or desiccant facilitated air-drying) can occur within about 1 hour.

In some embodiments, the polymer-based biological sample collection and/or separation devices described herein can be configured to enable lyophilization (e.g., lyophilization of a composition described herein, e.g., a polymer composition described herein) and/or a biological sample as described herein.

In some embodiments, the polymer-based biological sample collection and/or separation devices described herein can be configured to produce and/or to retain a substantially dried polymer composition as described herein (e.g., in the drying and recovery portion of the device), which can stabilizes, e.g., the structure, integrity, configuration, function, and/or activity of a biological sample, or at least one fraction, component, and/or analyte thereof for a period of time, e.g., during storage and transport of the biological sample. In some embodiments, the polymer-based biological sample collection and/or separation devices described herein can be configured to stabilize, e.g., the structure, integrity, configuration, function, and/or activity of a biological sample, or at least one fraction, component, and/or analyte thereof for at least about 1 day (e.g., about 1 day, about 2 days, about 3 days, about 4 days, about 5 days, about 6 days, about 7 days), e.g., at a temperature between about 4° C. and about 45° C. (e.g., about 4° C., about 10° C., about 15° C., about 20° C., about 25° C., about 30° C., about 35° C., about 37° C., about 40° C., or about 45° C.). In some embodiments, the polymer-based biological sample collection and/or separation devices described herein can be configured to stabilize, e.g., the structure, integrity, configuration, function, and/or activity of a biological sample, or at least one fraction, component, and/or analyte thereof for at least about 1 day (e.g., about 1 day, about 2 days, about 3 days, about 4 days, about 5 days, about 6 days, about 7 days, or longer), e.g., at a temperature between about 4° C. and about 45° C. (e.g., about 4° C., about 10° C., about 15° C., about 20° C., about 25° C., about 30° C., about 35° C., about 37° C., about 40° C., or about 45° C.). In some embodiments, the polymer-based biological sample collection and/or separation devices described herein can be configured to stabilize, e.g., the structure, integrity, configuration, function, and/or activity of a biological sample, or at least one fraction, component, and/or analyte thereof for at least about 1 week (e.g., about 1 week, about 2 weeks, about 3 weeks, about 1 month, about 2 months, about 3 months, about 4 months, about 5 months, about 6 months, about 7 months, about 8 months, about 9 months, about 10 months, about 11 months, about 1 year, or longer), e.g., at a temperature between about 4° C. and about 45° C. (e.g., about 4° C., about 10° C., about 15° C., about 20° C., about 25° C., about 30° C., about 35° C., about 37° C., about 40° C., or about 45° C.). In some embodiments, the polymer-based biological sample collection and/or separation devices described herein can be configured to stabilize the structure, integrity, configuration, function, and/or activity of a biological sample, or at least one fraction, component, and/or analyte thereof to enable long-term storage (e.g., for biobanking purposes), e.g., for at least about 1 year or longer, e.g., at a temperature between about 4° C. and about 45° C. (e.g., about 4° C., about 10° C., about 15° C., about 20° C., about 25° C., about 30° C., about 35° C., about 37° C., about 40° C., or about 45° C.). In some embodiments the drying may occur in a mixing and/or a drying and recovery portion of any one of the devices described herein.

In some embodiments, any of polymer-based biological sample collection and/or separation devices described herein can comprises about 0.01 mg to about 10 mg (e.g., about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, or about 10 mg) of a polymer composition as described herein, e.g., a silk fibroin composition. In some embodiments, the polymer-based biological sample collection and/or separation devices described herein can comprises 25 μL to about 300 μL (e.g., about 25 μL, about 30 μL, about 35 μL, about 40 μL, about 45 μL, about 50 μL, about 55 μL, about 60 μL, about 65 μL, about 70 μL, about 75 μL, about 80 μL, about 85 μL, about 90 μL, about 95 μL, about 100 μL, about 125 μL, about 150 μL, about 175 μL, about 200 μL, about 225 μL, about 250 μL, about 275 μL, about 300 μL) of a polymer composition as described herein, e.g., a silk fibroin composition.

In some embodiments, the polymer-based biological sample collection and/or separation devices described herein can comprises a polymer composition comprising about 0.01% w/v to about 50% w/v of a polymer (weight of the polymer with respect to the volume of sample) described herein (e.g., about 0.01% w/v, about 0.02% w/v, about 0.03% w/v, about 0.04% w/v, about 0.05% w/v, about 0.06% w/v, about 0.07% w/v, about 0.08% w/v, about 0.09% w/v, about 0.1% w/v, about 0.2% w/v, about 0.3% w/v, about 0.4% w/v, about 0.5% w/v, about 0.6% w/v, about 0.7% w/v, about 0.8% w/v, about 0.9% w/v, about 1% w/v, about 2% w/v, about 3% w/v, about 4% w/v, about 5% w/v, about 6% w/v, about 7% w/v, about 8% w/v, about 9% w/v, about 10% w/v, about 11% w/v, about 12% w/v, about 13% w/v, about 14% w/v, about 15% w/v, about 16% w/v, about 17% w/v, about 18% w/v, about 19% w/v, about 20% w/v, about 21% w/v, about 22% w/v, about 23% w/v, about 24% w/v, about 25% w/v, about 26% w/v, about 27% w/v, about 28% w/v, about 29% w/v, about 30% w/v, about 31% w/v, about 32% w/v, about 33% w/v, about 34% w/v, about 35% w/v, about 36% w/v, about 37% w/v, about 38% w/v, about 39% w/v, about 40% w/v, about 41% w/v, about 42% w/v, about 43% w/v, about 44% w/v, about 45% w/v, about 46% w/v, about 47% w/v, about 48% w/v, about 49% w/v, or about 50% w/v).

In some embodiments, the polymer can be a natural or a synthetic polymer. In some embodiments, the polymer is a peptide or a polypeptide. In some embodiments, the polymer comprises a silk fibroin protein, a gelatin protein, an albumin protein, an elastin protein, a collagen protein, a heparin protein, and/or a cellulose protein. In some embodiments, the polymer comprises polyethylene glycol (PEG), polyethylene oxide (PEO), hyaluronic acid, carboxymethylcellulose (CMC), polyvinylpyrrolidone (PVP), and/or polyvinyl alcohol (PVA).

In some embodiments, when the polymer composition is a silk fibroin composition, the polymer composition comprises about 0.1% w/v to about 20% w/v silk fibroin (weight of the silk fibroin with respect to the volume of sample) (e.g., about 0.01% w/v, about 0.02% w/v, about 0.03% w/v, about 0.04% w/v, about 0.05% w/v, about 0.06% w/v, about 0.07% w/v, about 0.08% w/v, about 0.09% w/v, about 0.1% w/v, about 0.2% w/v, about 0.3% w/v, about 0.4% w/v, about 0.5% w/v, about 0.6% w/v, about 0.7% w/v, about 0.8% w/v, about 0.9% w/v, about 1% w/v, about 2% w/v, about 3% w/v, about 4% w/v, about 5% w/v, about 6% w/v, about 7% w/v, about 8% w/v, about 9% w/v, about 10% w/v, about 11% w/v, about 12% w/v, about 13% w/v, about 14% w/v, about 15% w/v, about 16% w/v, about 17% w/v, about 18% w/v, about 19% w/v, or about 20%).

In some embodiments, when the polymer composition is a gelatin composition, the polymer composition comprises about 0.1% w/v to about 20% w/v gelatin (e.g., about 0.01% w/v, about 0.02% w/v, about 0.03% w/v, about 0.04% w/v, about 0.05% w/v, about 0.06% w/v, about 0.07% w/v, about 0.08% w/v, about 0.09% w/v, about 0.1% w/v, about 0.2% w/v, about 0.3% w/v, about 0.4% w/v, about 0.5% w/v, about 0.6% w/v, about 0.7% w/v, about 0.8% w/v, about 0.9% w/v, about 1% w/v, about 2% w/v, about 3% w/v, about 4% w/v, about 5% w/v, about 6% w/v, about 7% w/v, about 8% w/v, about 9% w/v, about 10% w/v, about 11% w/v, about 12% w/v, about 13% w/v, about 14% w/v, about 15% w/v, about 16% w/v, about 17% w/v, about 18% w/v, about 19% w/v, or about 20% w/v gelatin).

In some embodiments, the devices described herein can comprises about 200 μL of a silk fibroin composition, wherein the silk fibroin composition comprises about 4% w/v silk fibroin. In some embodiments, any of silk-based biological sample collection and/or separation devices described herein can comprises about 500 μL of a silk fibroin composition, wherein the silk fibroin composition comprises about 0.1% w/v silk fibroin. In some embodiments, the devices described herein can comprises about 50 μL of a silk fibroin composition, wherein the silk fibroin composition comprises about 2% w/v silk fibroin. In some embodiments, the devices described herein can comprises about 100 μL of a silk fibroin composition, wherein the silk fibroin composition comprises about 2% w/v silk fibroin. In some embodiments, the devices described herein can comprises about 150 μL of a silk fibroin composition, wherein the silk fibroin composition comprises about 2% w/v silk fibroin. In some embodiments, the devices described herein can comprises about 200 μL of a silk fibroin composition, wherein the silk fibroin composition comprises about 2% w/v silk fibroin.

In some embodiments, the polymer-based biological sample collection and/or separation devices described herein can comprises a silk fibroin composition comprising a population of silk fibroin having an average molecular weight of about 1 kDa to about 250 kDa (e.g., about 1 kDa to about 20 kDa, about 20 kDa to about 40 kDa, about 40 kDa to about 60 kDa, about 60 kDa to about 80 kDa, about 80 kDa to about 100 kDa, about 100 kDa to about 120 kDa, about 120 kDa to about 140 kDa, about 140 kDa to about 160 kDa, about 160 kDa to about 180 kDa, about 180 kDa to about 200 kDa). In some embodiments, the silk fibroin compositions is (i) a 10 minute boil (10 MB) silk fibroin composition, (ii) a 30 minute boil (30 MB) silk fibroin composition, (iii) a 60 minute boil (60 MB) silk fibroin composition, (iv) a 120 minute boil (120 MB) silk fibroin composition, (v) a 180 minute boil (180 MB) silk fibroin composition, (vi) a 240 minute boil (240 MB) silk fibroin composition, (vii) a 300 minute boil (300 MB) silk fibroin composition, (vii) a 360 minute boil (360 MB) silk fibroin composition, (ix) a 420 minute boil (420 MB) silk fibroin composition, and/or (x) a 480 minute boil (480 MB) silk fibroin composition, wherein the silk fibroin composition of (i)-(x) is produced accordingly to a method known in the art and/or described herein.

In some embodiments, the silk fibroin compositions is (i) the 30 MB silk fibroin composition comprises a population of silk fibroin having an average molecular weight of about 250 kDa (e.g., 246.9±16.9 kDa); (ii) the 60 MB silk fibroin composition is according to FIG. 42 and/or comprises a population of silk fibroin having an average molecular weight of about 150 kDa (e.g., 152.8±11.6 kDa); (iii) the 120 MB silk fibroin composition comprises a population of silk fibroin having an average molecular weight of about 100 kDa (e.g., 99.7±0.02 kDa); (iv) the 180 MB silk fibroin composition is according to FIG. 42 and/or comprises a population of silk fibroin having an average molecular weight of about 70 kDa (e.g., 70.5±0.38 kDa); and/or (v) the 480 MB silk fibroin composition is according to FIG. 42 and/or comprises a population of silk fibroin having an average molecular weight of about 36 kDa (e.g., 35.9±0.03 kDa).

In some embodiments, the polymer-based biological sample collection and/or separation devices described herein can comprises a silk fibroin composition comprising a population of silk fibroin having an average dispersity index (e.g., polydispersity index (PDI)) of about 5 to about 20 (e.g., about 5, about 6, about 7, about 8 about 9, about 10, about 11, about 12, about 13, about 14, about 15, about 16, about 17, about 18, about 19, about 20).

In some embodiments, the polymer-based biological sample collection and/or separation devices described herein can comprises a polymer composition, e.g., a silk fibroin composition, comprising an excipient. The excipient may be present in the device (e.g., in the polymer fibroin composition) in an amount less than 70% w/v (e.g., less than 70% w/v, less than 60% w/v, less than 50% w/v, less than 40% w/v, less than 30% w/v), less than 20% w/v, less than 10% w/v, less than 9% w/v, less than 8% w/v, less than 7% w/v, less than 6% w/v, 5% w/v, 1% w/v or less), optionally before drying. In some embodiments, the excipient is present in an amount between about 1% w/v to about 10% w/v (e.g., about 1% w/v, about 2% w/v, about 3% w/v, about 4% w/v, about 5% w/v, about 6% w/v, about 7% w/v, about 8% w/v, about 9% w/v, or about 10% w/v), optionally before drying. The excipient can be chosen from the group comprising a sugar and/or a sugar alcohol (e.g., sucrose, trehalose, maltose, sorbitol, mannitol, glycerol, or a combination thereof), a protein (e.g., a silk fibroin protein, a gelatin protein, an albumin protein, an elastin protein, a collagen protein, a heparin protein, and/or a cellulose protein), a polymer (e.g., polyethylene glycol (PEG), polyethylene oxide (PEO), hyaluroniacid, carboxymethylcellulose (CMC), polyvinylpyrrolidone (PVP), polyvinyl alcohol (PVA)), a divalent cation (e.g., Ca²⁺, Mg²⁺, Mn²⁺, and Cu²⁺), a salt (e.g., monosodium glutamate), a buffer (e.g., a Tris buffer, a Tris EDTA buffer, a citrate buffer, a phosphate buffer, a MOPS buffer, and/or a HEPES buffer), an amino acid (e.g., L-glutamiacid and/or lysine), and solvents (e.g., DMSO, methanol, and/or ethanol). In some embodiments, the excipient is present in the device (e.g., in the polymer composition) in an amount of about 1% (w/v). In some embodiments, the excipient is a sugar (e.g., sucrose).

In some embodiments, the device comprises a polymer composition comprising a divalent cation selected from the group consisting of Ca2+, Mg2+, Mn2+, and/or Cu2+, optionally wherein the divalent cation is present in an amount between 0.1 mM and 100 mM and/or between 10-10 to 2×10-3 moles.

In some embodiments, the device comprises a polymer fibroin composition comprising a buffer, optionally wherein the buffer has buffering capacity between pH 3 and pH 8, between pH 4 and pH 7.5, or between pH 5 and pH 7. The buffer can be chosen from the group comprising a Tris buffer, a Tris EDTA buffer, a citrate buffer, a phosphate buffer, a MOPS buffer, and a HEPES buffer.

In some embodiments, the device comprises a polymer composition comprising a buffer, optionally wherein the buffer is at a concentration of about 1 mM to about 300 mM (e.g., about 1 mM to about 10 mM, about 10 mM to about 20 mM, about 20 mM to about 30 mM, about 30 mM to about 40 mM, about 40 mM to about 50 mM, about 50 mM to about 60 mM about 60 mM to about 70 mM, about 70 mM to about 80 mM, about 80 mM to about 90 mM, about 90 mM to about 100 mM, about 100 mM to about 125 mM, about 125 mM to about 150 mM, about 150 mM to about 175 mM, about 175 mM to about 200 mM, about 200 mM to about 225 mM, about 225 mM to about 250 mM, about 250 mM to about 275 mM, or about 275 mM to about 300 mM). The buffer can be chosen from the group comprising a Tris buffer, a Tris EDTA buffer, a citrate buffer, a phosphate buffer, a MOPS buffer, and a HEPES buffer.

In some embodiments, any of polymer-based biological sample collection and/or separation devices described herein can comprise a lyophilized polymer composition as described herein, e.g., a lyophilized silk fibroin composition. In some embodiments, any of polymer-based biological sample collection and/or separation devices described herein can comprise a liquid polymer mixture. In some embodiments, any of polymer-based biological sample collection and/or separation devices described herein can comprise a substantially dried polymer composition (e.g., a lyophilized, an air-dried, or a spray dried polymer composition) as described herein.

In some embodiments, any of polymer-based biological sample collection and/or separation devices described herein can be used to collect a biological sample, optionally chosen from the group comprising a bodily fluid (e.g., whole blood, blood plasma, blood serum, urine, feces, saliva, amniotic fluid, and cerebrospinal fluid), cells and/or tissues (e.g., a buccal swab sample, a vaginal swab sample, and biopsy material, such as a tumor biopsy sample), and cell culture samples (e.g., eukaryotic cell culture samples and/or bacterial cell culture samples). In some embodiments, the biological sample is from a subject (e.g., a human subject).

In some embodiments, any of polymer-based biological sample collection and/or separation devices described herein can be configured to be compatible with any standard laboratory assays and/or instrumentation, e.g., for the analysis of a biological sample.

In one aspect, the invention features a method of making a substantially dried composition (e.g., a substantially dried polymer composition) as described herein, said method comprising the steps of: (i) metering a pre-determined amount of a biological sample (e.g., a whole blood, a blood serum, and/or blood plasma sample) into a device described herein, wherein the device comprises a polymer composition as described herein (e.g., a lyophilized silk fibroin composition); (ii) mixing (e.g., to homogeneity) the biological sample of (i) with the polymer composition of (i) to form a mixture; (iii) drying (e.g., air drying) the biological sample and/or the mixture, optionally, wherein the drying (e.g., air-drying and/or desiccant facilitated air-drying) of the biological sample and/or the mixture occurs within about 48 hours (e.g., within about 1-5 minutes, e.g., within about 1-10 minutes, e.g., within about 1-20 minutes, e.g., within about 1-30 minutes, e.g., within about 1-40 minutes, e.g., within about 1-50 minutes, e.g., within about 1-60 minutes, e.g., within about 1, 2, 3, 4, 5, 6, 7, 8, 9, 10, 20, 30, 40, 50, or 60 minutes, e.g., within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours), thereby making a substantially dried composition. In some embodiments, the substantially dried polymer composition can retain the structure, integrity, configuration, function, and/or activity of a biological sample, or at least one fraction, component, and/or analyte thereof, for at least 1 day (e.g., about 1 day, about 2 days, about 3 days, about 4 days, about 5 days, about 6 days, about 7 days), e.g., at a temperature between about 4° C. and about 45° C. (e.g., about 4° C., about 10° C., about 15° C., about 20° C., about 25° C., about 30° C., about 35° C., about 37° C., about 40° C., or about 45° C.). In some embodiments, the substantially dried polymer composition can retain the structure, integrity, configuration, function, and/or activity of a biological sample, or at least one fraction, component, and/or analyte thereof, for at least about 1 week (e.g., about 1 week, about 2 weeks, about 3 weeks, about 1 month, about 2 months, about 3 months, about 4 months, about 5 months, about six months, about 7 months, about 8 months, about 9 months, about 10 months, about 11 months, about 1 year, or longer) or longer, e.g., at a temperature between about 4° C. and about 45° C. (e.g., about 4° C., about 10° C., about 15° C., about 20° C., about 25° C., about 30° C., about 35° C., about 37° C., about 40° C., or about 45° C.).

In one aspect, the invention features a method of identifying and/or measuring the level of at least one fraction, component, and/or analyte of a biological sample, said method comprising the steps of: (i) metering a pre-determined amount of a biological sample (e.g., a whole blood, a blood serum, and/or blood plasma sample) into a device described herein, wherein the device comprises a polymer composition as described herein (e.g., a lyophilized silk fibroin composition); (ii) mixing (e.g., to homogeneity) the biological sample of (i) with the polymer composition of (i) to form a mixture; (iii) drying (e.g., air-drying and/or desiccant facilitated air-drying) the biological sample and/or the mixture, optionally, wherein the drying (e.g., air-drying and/or desiccant facilitated air-drying) of the biological sample and/or the mixture occurs within about 48 hours (e.g., within about 1-5 minutes, e.g., within about 1-10 minutes, e.g., within about 1-20 minutes, e.g., within about 1-30 minutes, e.g., within about 1-40 minutes, e.g., within about 1-50 minutes, e.g., within about 1-60 minutes, e.g., within about 1, 2, 3, 4, 5, 6, 7, 8, 9, 10, 20, 30, 40, 50, or 60 minutes, e.g., within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours), thereby making a substantially dried polymer composition; (iv) reconstituting the substantially dried polymer composition of (iii) in water, a buffer (e.g., PBS, optionally further comprising a surfactant and/or a sugar), an organic solvent, or a combination thereof to form an analyte sample (e.g., a reaction mixture and/or an analysis sample); (v) optionally, performing an extraction step on the analyte sample to form an extraction sample; (vi) optionally, performing a precipitation step on the analyte sample to form a precipitation sample; (vii) optionally, performing an enrichment step on the analyte sample to form an enrichment sample; (viii) optionally, performing a combination of steps (v)-(vii) to form a combination sample; and (ix) analyzing the analyte sample of (iv), the extraction sample of (v), the precipitation sample of (vi), the enrichment sample of (vii), and/or the combination sample of (viii) using standard laboratory assays and/or instrumentation as described herein, thereby identifying and/or measuring the level of at least one fraction, component, and/or analyte of a biological sample.

In some embodiments, the extraction step comprises: (i) a liquid-liquid extraction (e.g., for the extraction of analytes that are small molecules and/or enzymes); and/or (ii) a solid-liquid or solid-phase extraction (e.g., for the extraction of analytes that are small molecules and/or hormones).

In some embodiments, the enrichment step comprises a column enrichment, e.g., by an RNeasy spin column, when the analytes are nucleic acids such as mRNA and/or gDNA.

In some embodiments, the analyzing is by a clinical chemical analyzer. In some embodiments, the analyzing is by an LC-MS/MS instrument, an NMR instrument, and/or an rt-PCR instrument.

In one aspect, the invention features a substantially dried polymer composition made by the use of a device and/or a method as described herein. The substantially dried polymer composition can comprise about 0.01 mg to about 10 mg (e.g., about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, or about 10 mg) of a polymer as described herein, e.g., a silk fibroin protein, e.g., before drying. In some embodiments, the substantially dried polymer composition comprises 25 μL to about 300 μL (e.g., about 25 μL, about 30 μL, about 35 μL, about 40 μL, about 45 μL, about 50 μL, about 55 μL, about 60 μL, about 65 μL, about 70 μL, about 75 μL, about 80 μL, about 85 μL, about 90 μL, about 95 μL, about 100 μL, about 125 μL, about 150 μL, about 175 μL, about 200 μL, about 225 μL, about 250 μL, about 275 μL, about 300 μL) of a polymer composition described herein, e.g., before drying.

In some embodiments, the substantially dried composition comprises about 0.01% w/v to about 50% w/v of a polymer described herein (e.g., about 0.01% w/v, about 0.02% w/v, about 0.03% w/v, about 0.04% w/v, about 0.05% w/v, about 0.06% w/v, about 0.07% w/v, about 0.08% w/v, about 0.09% w/v, about 0.1% w/v, about 0.2% w/v, about 0.3% w/v, about 0.4% w/v, about 0.5% w/v, about 0.6% w/v, about 0.7% w/v, about 0.8% w/v, about 0.9% w/v, about 1% w/v, about 2% w/v, about 3% w/v, about 4% w/v, about 5% w/v, about 6% w/v, about 7% w/v, about 8% w/v, about 9% w/v, about 10% w/v, about 11% w/v, about 12% w/v, about 13% w/v, about 14% w/v, about 15% w/v, about 16% w/v, about 17% w/v, about 18% w/v, about 19% w/v, about 20% w/v, about 21% w/v, about 22% w/v, about 23% w/v, about 24% w/v, about 25% w/v, about 26% w/v, about 27% w/v, about 28% w/v, about 29% w/v, about 30% w/v, about 31% w/v, about 32% w/v, about 33% w/v, about 34% w/v, about 35% w/v, about 36% w/v, about 37% w/v, about 38% w/v, about 39% w/v, about 40% w/v, about 41% w/v, about 42% w/v, about 43% w/v, about 44% w/v, about 45% w/v, about 46% w/v, about 47% w/v, about 48% w/v, about 49% w/v, or about 50% w/v). In some embodiments, the polymer can be a natural and/or a synthetic polymer. In some embodiments, the polymer can be a peptide or a polypeptide. In some embodiments, the polymer comprises a silk fibroin protein, a gelatin protein, an albumin protein, an elastin protein, a collagen protein, a heparin protein, and/or a cellulose protein. In some embodiments, the polymer comprises polyethylene glycol (PEG), polyethylene oxide (PEO), hyaluronic acid, carboxymethylcellulose (CMC), polyvinylpyrrolidone (PVP), and/or polyvinyl alcohol (PVA).

In particular embodiments, the substantially dried polymer composition comprises about 200 μL of a silk fibroin composition, wherein the silk fibroin composition comprises about 4% w/v silk fibroin, e.g., before drying. In particular embodiments, the substantially dried polymer composition comprises about 500 μL of a silk fibroin composition, wherein the silk fibroin composition comprises about 0.1% w/v silk fibroin, e.g., before drying.

The substantially dried polymer composition can comprises a silk fibroin composition comprising a population of silk fibroin having an average molecular weight of about 1 kDa to about 250 kDa (e.g., about 1 kDa to about 20 kDa, about 20 kDa to about 40 kDa, about 40 kDa to about 60 kDa, about 60 kDa to about 80 kDa, about 80 kDa to about 100 kDa, about 100 kDa to about 120 kDa, about 120 kDa to about 140 kDa, about 140 kDa to about 160 kDa, about 160 kDa to about 180 kDa, about 180 kDa to about 200 kDa), e.g., before drying. In some embodiments, the substantially dried composition can comprise (i) a 10 minute boil (10 MB) silk fibroin composition, (ii) a 30 minute boil (30 MB) silk fibroin composition, (iii) a 60 minute boil (60 MB) silk fibroin composition, (iv) a 120 minute boil (120 MB) silk fibroin composition, (v) a 180 minute boil (180 MB) silk fibroin composition, (vi) a 240 minute boil (240 MB) silk fibroin composition, (vii) a 300 minute boil (300 MB) silk fibroin composition, (vii) a 360 minute boil (360 MB) silk fibroin composition, (ix) a 420 minute boil (420 MB) silk fibroin composition, and/or (x) a 480 minute boil (480 MB) silk fibroin composition, wherein the silk fibroin composition of (i)-(x) is produced accordingly to a method known in the art and/or described herein. In some embodiments, (i) the 30 MB silk fibroin composition comprises a population of silk fibroin having an average molecular weight of about 250 kDa (e.g., 246.9±16.9 kDa); (ii) the 60 MB silk fibroin composition is according to FIG. 42 and/or comprises a population of silk fibroin having an average molecular weight of about 150 kDa (e.g., 152.8±11.6 kDa); (iii) the 120 MB silk fibroin composition comprises a population of silk fibroin having an average molecular weight of about 100 kDa (e.g., 99.7±0.02 kDa); (iv) the 180 MB silk fibroin composition is according to FIG. 42 and/or comprises a population of silk fibroin having an average molecular weight of about 70 kDa (e.g., 70.5±0.38 kDa); and/or (v) the 480 MB silk fibroin composition is according to FIG. 42 and/or comprises a population of silk fibroin having an average molecular weight of about 36 kDa (e.g., 35.9±0.03 kDa).

The substantially dried polymer composition can comprise a population of silk fibroin having an average dispersity index (e.g., polydispersity index (PDI)) of about 5 to about 20 (e.g., about 5, about 6, about 7, about 8 about 9, about 10, about 11, about 12, about 13, about 14, about 15, about 16, about 17, about 18, about 19, about 20), e.g., before drying.

The substantially dried polymer composition can comprise an excipient, e.g., before drying. The excipient can be chosen from the group comprising a sugar and/or a sugar alcohol (e.g., sucrose, trehalose, sorbitol, mannitol, glycerol, or a combination thereof), a protein excipient (e.g., gelatin, albumin, elastin, heparin, and/or collagen), a divalent cation (e.g., Ca2+, Mg2+, Mn2+, and Cu2+), a salt (e.g., monosodium glutamate), a buffer, an amino acid (e.g., L-glutamic acid and/or lysine), and solvents (e.g., DMSO, methanol, and/or ethanol). The excipient can be present in an amount less than 70% w/v (e.g., less than 70% w/v, less than 60% w/v, less than 50% w/v, less than 40% w/v, less than 30% w/v), less than 20% w/v, less than 10% w/v, less than 9% w/v, less than 8% w/v, less than 7% w/v, less than 6% w/v, 5% w/v, 1% w/v or less), e.g., before drying. In some embodiments, the excipient is present in an amount between about 1% w/v to about 10% w/v (e.g., about 1% w/v, about 2% w/v, about 3% w/v, about 4% w/v, about 5% w/v, about 6% w/v, about 7% w/v, about 8% w/v, about 9% w/v, or about 10% w/v), e.g., before drying. In some embodiments, the excipient can be present in an amount of about 1% (w/v), e.g., before drying. In some embodiments, the excipient can be present in an amount between 0.1 mM and 100 mM and/or between 10⁻¹⁰ to 2×10⁻³ moles, e.g., before drying. In particular embodiments, the excipient is a sugar (e.g., sucrose).

The substantially dried polymer composition can further comprise a buffer (e.g., a buffer salt), before drying. The buffer can have a buffering capacity between pH 3 and pH 8, between pH 4 and pH 7.5, or between pH 5 and pH 7, e.g., before drying. In some embodiments, the buffer is at a concentration of about 1 mM to about 300 mM (e.g., about 1 mM to about 10 mM, about 10 mM to about 20 mM, about 20 mM to about 30 mM, about 30 mM to about 40 mM, about 40 mM to about 50 mM, about 50 mM to about 60 mM about 60 mM to about 70 mM, about 70 mM to about 80 mM, about 80 mM to about 90 mM, about 90 mM to about 100 mM, about 100 mM to about 125 mM, about 125 mM to about 150 mM, about 150 mM to about 175 mM, about 175 mM to about 200 mM, about 200 mM to about 225 mM, about 225 mM to about 250 mM, about 250 mM to about 275 mM, or about 275 mM to about 300 mM). Optionally, the buffer can be chosen from the group comprising a Tris buffer, a Tris EDTA buffer, a citrate buffer, a phosphate buffer, a MOPS buffer, and a HEPES buffer.

The substantially dried polymer composition described herein can stabilize the structure, integrity, configuration, function, and/or activity of a biological sample, or at least one fraction, component, and/or analyte thereof for at least about 1 day (e.g., about 1 day, about 2 days, about 3 days, about 4 days, about 5 days, about 6 days, about 7 days, or longer), e.g., at a temperature between about 4° C. and about 45° C. (e.g., about 4° C., about 10° C., about 15° C., about 20° C., about 25° C., about 30° C., about 35° C., about 37° C., about 40° C., or about 45° C.). In some embodiments, substantially dried polymer composition described herein can stabilize the structure, integrity, configuration, function, and/or activity of a biological sample, or at least one fraction, component, and/or analyte thereof for at least about 1 week (e.g., about 1 week, about 2 weeks, about 3 weeks, about 1 month, about 2 months, about 3 months, about 4 months, about 5 months, about six months, about 7 months, about 8 months, about 9 months, about 10 months, about 11 months, about 1 year, or longer), e.g., at a temperature between about 4° C. and about 45° C. (e.g., about 4° C., about 10° C., about 15° C., about 20° C., about 25° C., about 30° C., about 35° C., about 37° C., about 40° C., or about 45° C.). In some embodiments, the substantially dried polymer composition described herein can stabilize the structure, integrity, configuration, function, and/or activity of a biological sample, or at least one fraction, component, and/or analyte thereof to enable long-term storage (e.g., for biobanking purposes). In particular embodiments, the substantially dried polymer composition described herein can stabilize the structure, integrity, configuration, function, and/or activity of a biological sample, or at least one fraction, component, and/or analyte thereof for at least about 1 year or longer, e.g., at a temperature between about 4° C. and about 45° C. (e.g., about 4° C., about 10° C., about 15° C., about 20° C., about 25° C., about 30° C., about 35° C., about 37° C., about 40° C., or about 45° C.).

In one aspect, the invention features a mixture (e.g., a polymer mixture for drying) made by the use of a device and/or a method described herein.

In one aspect, the invention features a reaction mixture and/or an analysis sample made by reconstituting a substantially dried polymer composition ad described herein and made by the use of a device and/or a method described herein. In some embodiments, the reaction mixture can be made by reconstituting a substantially dried polymer composition as described herein in a reconstitution solution, e.g., in water, a buffer (e.g., a sample buffer, such as phosphate buffered saline with added tween and/or sugars), and/or in a solvent (e.g., methanol, ethanol, acetone, acetonitrile, chloroform, trifluoroacetic acid (TFA), and/or formic acid (FA), e.g., a solvent comprising 0.1% TFA in acetonitrile, 0.1% TFA in water, 0.1% FA in acetonitrile, or 0.1% FA in water), and which is compatible is standard laboratory assays and/or instrumentation.

In one aspect, the invention features a kit comprising a device as described herein. Optionally, the kit may further comprise a capillary tube (e.g., to be used for metering of the biological sample, e.g., outside the device), an metering device (e.g., a syringe, a cuvette, and/or a pipette), a lancet or other form of a venipuncture device, a bandage for wound closure, a bag to contain sample and/or to ship sample, an additional amount of a desiccant described herein, a gauze to wipe initial blood drop, a biohazard sealable foil pouch/bag (e.g., for storage and/or shipping), and/or instructions for use.

Those skilled in the art will recognize, or be able to ascertain using no more than routine experimentation, many equivalents to the specific embodiments of the invention described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. Various aspects of at least one embodiment are discussed below with reference to the accompanying figures, which are not intended to be drawn to scale. Where technical features in the figures, detailed description or any claim are followed by reference signs, the reference signs have been included for the sole purpose of increasing the intelligibility of the figures, detailed description, and claims. Accordingly, neither the reference signs nor their absence are intended to have any limiting effect on the scope of any claim elements. In the figures, each identical or nearly identical component that is illustrated in various figures is represented by a like numeral. For purposes of clarity, not every component may be labeled in every figure. The figures are provided for the purposes of illustration and explanation and are not intended as a definition of the limits of the disclosure. In the figures:

FIG. 1 is a perspective view of a collection device of an embodiment of the present disclosure.

FIG. 2 is a perspective view of the collection device shown in FIG. 1 having transparent packaging to reveal component parts of the collection device.

FIG. 3 is a perspective view of a collection device of another embodiment of the present disclosure.

FIG. 4 is a cross-sectional perspective view of the collection device shown in FIG. 3.

FIG. 5 is an exploded perspective view of the collection device shown in FIGS. 3 and 4.

FIG. 6 is a perspective view of a plasma separation card of an embodiment of the present disclosure.

FIG. 7 is an exploded perspective view of the plasma separation card shown in FIG. 6.

FIG. 8 is a perspective view of a sample separation accessory of an embodiment of the present disclosure.

FIG. 9 is an exploded perspective view of the sample separation assembly shown in FIG. 8.

FIG. 10 illustrates two embodiments of a plasma separation membrane with polymer formulation dried into the membrane.

FIG. 11 illustrates a sample collection device of an embodiment of the disclosure.

FIG. 12 illustrates a sample collection device of an embodiment of the disclosure.

FIGS. 13-22 illustrate a sample collection device of an embodiment of the disclosure.

FIG. 23 illustrates a sample collection device of an embodiment of the disclosure.

FIG. 24 illustrates a sample collection device of an embodiment of the disclosure.

FIGS. 25-28 illustrate well structures of embodiments of the disclosure used in a sample collection device.

FIGS. 29-32 illustrate a sample collection device of an embodiment of the disclosure.

FIGS. 33-39 illustrate a sample collection device of an embodiment of the disclosure.

FIG. 40 illustrates a sample collection device of an embodiment of the disclosure.

FIGS. 41A-41E illustrate kit packaging of embodiments of the disclosure.

FIG. 42 is a series of charts that illustrate various molecular weight profiles of silk fibroin solutions useful in fabricating a silk-based biological sample collection device described herein.

FIG. 43 is an image showing 1% (w/v), 2% (w/v), and 4% (w/v) silk fibroin compositions pipetted into plastic capillary tubes having an internal diameter of 1.75 mm. The indicated volume of lyophilized silk was mixed with the indicated volume of blood to produce the 1% (w/v), 2% (w/v), and 4% (w/v) silk fibroin composition. Scale bar=10 mm.

FIG. 44A is a chart showing the rate of water loss from an initial 100 μL volume of 1% w/v silk casted on a polydimethylsiloxane (PDMS) disk of diameter 10 mm, 12 mm, or 14 mm. The PDMS disk was positioned on top of the indicated mass of molecular sieve desiccant inside a sealed 20 mL scintillation vial over 8-hours. The rate of water loss was calculated via linear regression. The data show that the 100 μL volume of 1% w/v silk dried significantly faster with larger casting surface areas, however, no significant increase in drying rate with increasing the desiccant amount from 2.5 g to 10 g. Two-way ANOVA with Tukey's post-bioanalysis used to determine significant difference (bars indicates p<0.05 between groups).

FIG. 44B is a chart showing the kinetics of drying from an initial 100 μL volume of 1% w/v silk casted on a polydimethylsiloxane (PDMS) disk of diameter 10 mm, 12 mm, or 14 mm. The PDMS disk was positioned on top of 2.5 g of molecular sieve desiccant, and stored inside a sealed 20 mL scintillation vial. Samples were weighed each hour until fully dried to determine the percent water loss from the sample. All samples containing 2.5 grams desiccant were fully dry within 6 hours. Two-way ANOVA with Tukey's post-bioanalysis used to determine significant difference (bars indicates p<0.05 between groups).

FIG. 45A is a chart showing the analyte recovery of total cholesterol (CHOL), high-density lipoprotein (HDL), triglyceride (TRIG), alkaline phosphatase (ALP), aspartate transaminase (AST), and alanine transaminase (ALT) concentrations in formulated plasma. Lithium heparinized plasma was used to reconstitute listed formulations and run on a Beckman Coulter AU680 chemistry analyzer. Data is normalized to baseline plasma analyte levels.

FIG. 45B is a series of charts showing the analyte alkaline phosphatase (ALP) and aspartate transaminase (AST) concentrations in formulated plasma films. Lithium heparinized plasma was used to reconstitute listed formulations and dried onto a 12 mm polydimethylsiloxane disk. Once dried, samples were transferred to 4° C. overnight. For analysis, samples were reconstituted with deionized water and run on a Beckman Coulter AU680 chemistry analyzer. Data is normalized to baseline liquid plasma analyte levels. One-way ANOVA with Dunnett's post-bioanalysis used to determine significant difference between formulated and non-formulated films (bars indicates p<0.05 between groups).

FIG. 46A is a series of graphs showing the stability of aKG in serum, dried serum spots (DSS), film A (2% silk), and film B (2% silk, 1% sucrose) at 4° C., 22° C., 37° C., and 45° over 7 days.

FIG. 46B shows that DSS overestimates concentration of aKG, presumably due to approximation of total serum amount in punch and that silk film recoveries were similar between Film A and Film B (left panel); and that silk film (2% silk, 1% sucrose) outperforms DBS stability profile by 20% (right panel).

FIG. 47A is a chart showing the analyte recovery of total cholesterol (CHOL), high-density lipoprotein (HDL), triglyceride (TRIG), alanine transaminase (ALT), gamma-glutamyl transferase (GGT), and C-reactive protein (CRP) concentrations in formulated plasma. EDTA anticoagulated plasma was used to reconstitute listed formulations and run on a Beckman Coulter AU680 chemistry analyzer. Data is normalized to baseline plasma analyte levels.

FIG. 47B is a chart showing the analyte recovery of gamma-glutamyl transferase (GGT, and C-reactive protein (CRP) in formulated plasma films. EDTA anticoagulated plasma was used to reconstitute listed formulations and dried onto a polydimethylsiloxane disk. Once dried, samples were transferred to 37° for 5 days. For analysis, samples were reconstituted with deionized water and run on a Beckman Coulter AU680 chemistry analyzer. Data is normalized to baseline plasma analyte levels. Films with formulation showed an enhanced protection compared to the control film. One-way ANOVA with Dunnett's post-bioanalysis used to determine significant difference between formulated and non-formulated films (bars indicates p<0.05 between groups). Additionally, a t-test was used to determine significant differences between the control liquid plasma and film groups (* indicates p<0.05 against control liquid).

FIG. 47C is a chart showing the alanine transaminase (ALT) concentrations in formulated plasma films. EDTA and lithium heparin anticoagulated plasma was used to reconstitute listed formulations and dried onto a polydimethylsiloxane disk. Once dried, samples were transferred to 4° for 5 days. For analysis, samples were reconstituted with deionized water and run on a Beckman Coulter AU680 chemistry analyzer. Data is normalized to baseline plasma analyte levels. One-way ANOVA with Dunnett's post-bioanalysis used to determine significant difference between formulated and non-formulated films (bars indicates p<0.05 between groups).

FIG. 48 is an image showing formulation compositions containing silk, sucrose, or HEPES after being pipetted and lyophilized into plastic capillary tubes having an internal diameter of 2.35 mm. Tubes were weighed before and after lyophilization to determine approximate moisture content within the tubes.

FIG. 49 is an image showing formulated whole blood applied to lateral flow separation membranes. Lithium heparinized whole blood was mixed with formulation and pipetted onto the side of a lateral flow strips (AdvanceDX (ADX100) or GE Healthcare's LF1 paper). The presence of silk formulation impedes flow of red blood cells, leaving a larger plasma/serum area. Length of paper=64 mm.

FIGS. 50A-50B are graphs showing pH kinetics as a plasma film dries over the course of 3 hours. Lithium heparinized plasma was mixed with MOPS or HEPES buffer to yield a final buffer concentration ranging from 0-100 mM (FIG. 50A). Samples were cast onto 16 mm polydimethylsiloxane disks and dried. Once dried, films were transferred to tubes and reconstituted with deionized water. The pH of the plasma was measured throughout the drying process and post-reconstitution (FIG. 50B).

FIGS. 51A-51B are graph showing alkaline phosphatase (ALP) HEPES buffer assay interference in liquid plasma (FIG. 51A) and recovery in plasma films when formulated with HEPES buffer at different concentrations (FIG. 51B). Lithium heparinized plasma was mixed with HEPES buffer before being cast and dried onto 16 mm polydimethylsiloxane disks. Once dried, samples were immediately reconstituted with deionized water and assayed for ALP concentration using a plate reader assay (Biovision, Cat. #: K412). Data is normalized to neat liquid plasma. ANOVA with Dunnett's post-bioanalysis used to determine significant significance (bars indicates p<0.05 compared to neat liquid plasma).

FIGS. 52A-52B are graphs showing the relation between alkaline phosphatase (ALP) recovery and total percent solids loading in plasma films. Lithium heparinized plasma was mixed with different ratios of gelatin, sucrose, and HEPES, before being cast and dried onto 16 mm polydimethylsiloxane disks. Once dried, samples were transferred to 37° overnight. For analysis, samples were reconstituted with deionized water and assayed on a plate reader ALP assay (Biovision, Cat. #: K412). FIG. 52A: Data is normalized to Day 0 liquid plasma baseline measurements. FIG. 52B: A sigmoidal 4-parameter logistic model was used to fit the data to a non-linear regression model.

FIGS. 53A-53B is a series of charts showing the analyte recovery of albumin (ALB), total protein (TP), creatinine (CREAT), blood urea nitrogen (BUN), bilirubin (BIL), calcium (CA), chloride (CL), sodium (NA), potassium (K), bicarbonate (CO2), glucose (GLUC), alkaline phosphatase (ALP), alanine transaminase (ALT), and aspartate transaminase (AST), triglycerides (TRIG), and c-reactive protein (CRP) concentrations in formulated plasma. Lithium heparinized plasma was used to reconstitute lyophilized formulations and run on a Beckman Coulter AU680 chemistry analytes (FIG. 53A) or a Cobas8000 series instrument (FIG. 53B). Data is normalized to baseline liquid plasma analyte levels. T-test analysis was used to determine significance (bars indicate p<0.05 compared to control).

FIG. 54 is a chart showing the analyte recovery of albumin (ALB), total protein (TP), creatinine (CREAT), blood urea nitrogen (BUN), bilirubin (BIL), calcium (CA), chloride (CL), sodium (NA), potassium (K), bicarbonate (CO2), glucose (GLUC), and alkaline phosphatase (ALP) concentrations in formulated and non-formulated plasma films. Lithium heparinized plasma was used to reconstitute listed formulations and dried onto a 16 mm polydimethylsiloxane disk. Once dried, samples were transferred to 4° overnight. For analysis, samples were reconstituted with deionized water back to the neat plasma concentration and run on a Beckman Coulter AU680 chemistry analyzer. Data is normalized to baseline liquid plasma analyte levels. ANOVA with Dunnett's post-bioanalysis used to determine significant significance (bars indicates p<0.05 compared to neat liquid plasma).

FIG. 55 is a series of charts showing the analyte recovery of aspartate transaminase (AST) and alanine transaminase (ALT) concentrations in neat liquid plasma, plasma dried on Whatman 903 paper (Dried Plasma Spot, DPS), and formulated plasma films (Formulation). For DPS samples, lithium heparinized plasma was dried on 16 mm punches of Whatman 903 paper. For formulated films, plasma used to reconstitute formulation and dried onto a 16 mm polydimethylsiloxane disk. Once dried, samples were transferred to 4° or 37° for stability assessment. For analysis, DPS samples were eluted in deionized water for 1 hour and formulated samples were reconstituted with deionized water back to the neat plasma concentration. All samples were analyzed on a Beckman Coulter AU680 chemistry analyzer. Data is normalized to Day 1 frozen plasma analyte levels. ANOVA with Dunnett's post-bioanalysis used to determine significance against neat liquid plasma (bars indicates p<0.05 compared to neat liquid plasma), and t-test used to determine significance against Day 1 for respective format (* indicate p<0.05).

FIG. 56 is a series of charts showing the analyte recovery of cardiovascular marker analytes total cholesterol (CHOL), high-density lipoproteins (HDL), triglycerides (TRIG), and c-reactive protein (CRP) concentrations in neat liquid plasma, air-dried plasma films (non-formulated), and formulated plasma films. Plasma was used to reconstitute formulation and dried onto 16 mm polydimethylsiloxane disks. Once dried, samples were transferred to 37° or 45° for 5 days for stability assessment. For analysis, samples were reconstituted with deionized water back to the neat plasma concentration. All samples were analyzed on Cobas 8000 series instrumentation. Samples were compared to neat liquid plasma, and normalized to −80° stored plasma. ANOVA with Dunnett's post-bioanalysis was used to determine significance against neat liquid plasma (bars indicate p<0.05), and t-tests were used to determine significance against −80° stored plasma (* indicate p<0.05).

FIGS. 57A-57C is a series of images and charts showing the behavior and separation of whole blood on GE Healthcare's LF1 paper, and Ahlstrom-Munksjö's 1660 and 1667 lateral flow paper. Neat blood (Control) or formulated blood was blotted onto the end of the strips of paper and allowed to dry. FIG. 57A: Representative images of membranes post-separation and fully dried. FIG. 57B: Image analysis was used to determine total areas of the red blood cell and plasma region of test strips. T-tests were used to determine significance between groups (bars indicate p<0.05. FIG. 57C: Mass of dried plasma/area of isolated plasma fragments of paper. T-tests were used to determine significance between groups (bars indicate p<0.05).

FIGS. 58A-58C is a series of charts showing the behavior and separation of whole blood on GE Healthcare's LF1 paper when formulated with silk, sucrose, HEPES, gelatin, and/or bovine serum albumin (BSA). Whole blood was used to reconstitute lyophilized formulations before being pipetted onto the paper membranes. Membranes were allowed to separate and dry overnight. FIG. 58A-58B: Image analysis was used to determine total areas of the red blood cell and plasma region of test strips. ANOVA with Dunnett's post-bioanalysis was used to determine significance against non-formulated groups (bars indicate p<0.05). FIG. 58C: Mass of dried plasma/area of isolated plasma fragments of paper. ANOVA with Dunnett's post-bioanalysis was used to determine significance against non-formulated groups (bars indicate p<0.05).

FIG. 59 is a series of charts showing the mass of dried formulation after spread onto GE Healthcare's LF1 paper. Deionized water was used to reconstitute silk, sucrose, and/or HEPES formulations before being pipetted onto the end of the membranes. The charts above show a heat map of the masses along the membrane, with the formulation being deposited on the left end of the strip.

FIG. 60 is a series of charts showing the analyte recovery of liver enzymes aminotransferase (AST) and alanine aminotransferase (ALT) in neat liquid plasma, air-dried plasma (non-formulated films), AdvanceDX 100, and formulated films. Samples were stored at 4° C. or 37° C. for 3 days. Percent recovery samples were compared 4° C. stored plasma. N=3 replicates per donor.

### DETAILED DESCRIPTION OF THE DISCLOSURE

The present disclosure is directed to methods and devices used to simplify the collection, shipment and storage of biological samples (e.g., bodily fluids, such as whole blood, blood plasma, and/or blood serum). Specifically, a polymer composition (e.g., a silk fibroin composition) is provided to include a formulation and drying platform to reduce or eliminate the need for cold storage and shipment of a biological sample, including a blood sample, e.g., whole blood, serum, and/or plasma. With this platform, a biological sample, e.g., a blood sample, is deposited on and mixed with a polymer composition (e.g., a polymer matrix, such as a lyophilized silk fibroin composition), dried (e.g., air-dried) at room temperature, shipped and re-solubilized for analysis at a final destination, e.g., using standard laboratory assays and/or instrumentation. In one embodiment, silk can be used as a standalone substrate and/or integrated into an existing platform, e.g., DBS cards, so as not to interfere with downstream analytical tools. Further examples of devices used to collect, store and transport blood samples are contemplated. One set of devices employ methods that use paper and takes advantage of lateral flow. Another set of devices uses free film and takes advantage of vertical flow.

### Definitions

All scientific and technical terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent or later-developed techniques which would be apparent to one of skill in the art. In addition, in order to more clearly and concisely describe the subject matter which is the invention, the following definitions are provided for certain terms which are used in the specification and appended claims.

The articles “a” and “an” are used herein to refer to one or to more than one (i.e., to at least one) of the grammatical object of the article. By way of example, “an element” means one element or more than one element.

As used herein, the term “about” means +/−10% of the recited value.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

As used herein, the term “gelatin” refers to a water-soluble protein derived from collagen. In some embodiments, the term “gelatin” refers to a sterile nonpyrogenic protein preparation (e.g., fractions) produced by partial acid hydrolysis (type A gelatin) or by partial alkaline hydrolysis (type B gelatin) of animal collagen, most commonly derived from cattle, pig, and fish sources. Gelatin can be obtained in varying molecular weight ranges. Recombinant sources of gelatin may also be used.

As used herein, the term “silk fibroin” includes silkworm fibroin and insect or spider silk protein. Any type of silk fibroin can be used according to various aspects described herein. Silk fibroin produced by silkworms, such as Bombyx mori, is the most common and represents an earth-friendly, renewable resource. For instance, silk fibroin used in silk fibroin composition as described herein, e.g. may be obtained by removing sericin from the cocoons of B. mori. In some embodiments, the silk fibroin is a regenerated silk fibroin, e.g., a silk fibroin obtained after extraction of sericin from the cocoons of B. mori, and an additional processing e.g. via a boiling step. Organic silkworm cocoons are also commercially available. There are many different silks, however, including spider silk (e.g., obtained from Nephila clavipes), transgenic silks, recombinant and/or genetically engineered silks, such as silks from bacteria, yeast, mammalian cells, transgenic animals, or transgenic plants (see, e.g., WO 97/08315; U.S. Pat. No. 5,245,012), and variants thereof, that can be used.

As used herein, the term “silk fibroin fragments” refers to peptide chains or polypeptides having an amino acid sequence corresponding to fragments derived from silk fibroin protein or variants thereof. In some embodiments, the term “silk fibroin fragments” refers to fibroin peptide chains or polypeptides that are smaller than the naturally occurring full length silk fibroin counterpart, such that one or more of the silk fibroin fragments within a population or composition are less than 300 kDa, less than 250 kDa, less than 200 kDa, less than 175 kDa, less than 150 kDa, less than 120 kDa, less than 100 kDa, less than 90 kDa, less than 80 kDa, less than 70 kDa, less than 60 kDa, less than 50 kDa, less than 40 kDa, less, than 30 kDa, less than 25 kDa, less than 20 kDa, less than 15 kDa, less than 12 kDa, less than 10 kDa, less than 9 kDa, less than 8 kDa, less than 7 kDa, less than 6 kDa, less than 5 kDa, less than 4 kDa, or less than 3.5 kDa.

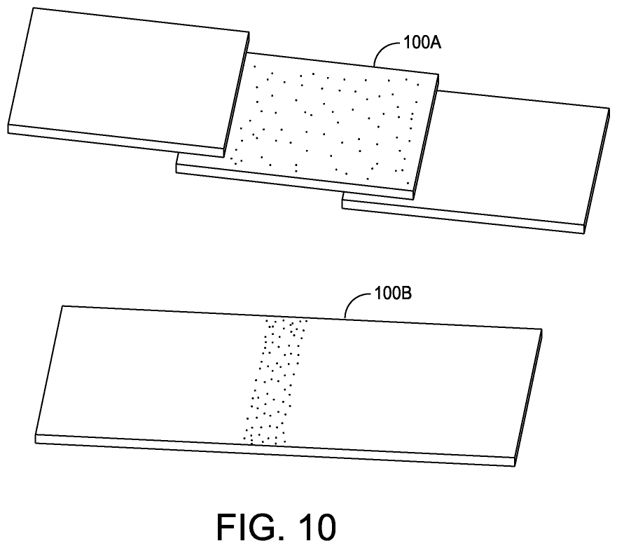

In some embodiments, the silk fibroin fragments can be derived by degumming silk cocoons, e.g., under conditions that remove sericin, e.g., as a step in the process used to produce silk fibroin fragments having the desired range of molecular weights and/or molecular weight profile, e.g., as shown in FIG. 10 or described herein. In some embodiments, the silk fibroin compositions described herein comprise less than about 5% sericin w/w.

In some embodiments, silk fibroin fragments can be derived by degumming silk cocoons at or close to (e.g., within 5% around) an atmospheric boiling temperature and/or in an aqueous solution at about 90° C. to about 110° C. (e.g., 90° C., 91° C., 92° C., 93° C., 94° C., 95° C., 96° C., 97° C., 98° C., 99° C., 100° C., 102° C., 103° C., 104° C., 105° C., 106° C., 107° C., 108° C., 109° C., or 110° C.) for at least about 10 minutes or longer (e.g., about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, about 500 minutes).

As used herein, the terms “stabilizing,” “stabilize,” “stability,” and “stabilization,” refer to retaining the structure, integrity, configuration, function, and/or activity of a biological sample, or at least one analyte thereof, as described herein, such that the biological sample, or at least one analyte thereof, displays less degradation, loss (e.g., loss of quantity), misfolding, denaturation, aggregation, and/or inactivation by use of a silk-based biological sample collection device as described herein. In some embodiments, at least one analyte of a biological sample can be stabilized within a silk-based biological sample collection device as described herein such that, e.g., an activity, structural feature, or pre-determined amount (e.g., a detectable amount) of the at least one analyte is partially or completely maintained in a silk-based biological sample collection device over a period of time, e.g., as determined by standard laboratory methods.

In some embodiments, the silk-based biological sample collection devices described herein can be used, e.g., to stabilize a biological sample, a fraction, a component, and/or an analyte thereof, for at least about 1 day (e.g., about 1 day, about 2 days, about 3 days, about 4 days, about 5 days, about 6 days, about 7 days), e.g., at a temperature between about 4° C. and about 45° C. (e.g., about 4° C., about 10° C., about 15° C., about 20° C., about 25° C., about 30° C., about 35° C., about 37° C., about 40° C., or about 45° C.). In some embodiments, the silk-based biological sample collection devices described herein can be used, e.g., to stabilize a biological sample, a fraction, a component, and/or an analyte thereof, for at least about 1 week, e.g., about 1 week to about 2 weeks (e.g., 1, 2, 3, 4, 5, 6, 7, 8, 9, 10, 11, 12, 13, or 14 days) at a temperature between about 4° C. and about 45° C. (e.g., about 4° C., about 10° C., about 15° C., about 20° C., about 25° C., about 30° C., about 35° C., about 37° C., about 40° C., or about 45° C.), as further described herein.

With respect to stabilization of an activity and/or a function, in some embodiments, at least about 10% or more (e.g., at least about 10%, at least about 20%, at least about 30%, at least about 40%, at least about 50%, at least about 60%, at least about 70%, at least about 80%, at least about 90% or more, e.g., up to about 100%) of a biological sample, or at least one analyte thereof, can retain at least a partial or complete activity and/or function over a period of time, e.g., compared to a reference sample.

With respect to stabilization of structure, configuration, and/or integrity, in some embodiments, at least about 10% or more (e.g., at least about 10%, at least about 20%, at least about 30%, at least about 40%, at least about 50%, at least about 60%, at least about 70%, at least about 80%, at least about 90% or more, e.g., up to about 100%) of a biological sample, or at least one analyte thereof, can retain its structure, configuration, and/or integrity over a period of time, e.g., compared to a reference sample.

With respect to stabilization of quantity (e.g., a detectable amount and/or measurable amount), in some embodiments, at least about 10% or more (e.g., at least about 10%, at least about 20%, at least about 30%, at least about 40%, at least about 50%, at least about 60%, at least about 70%, at least about 80%, at least about 90% or more, e.g., up to about 100%) of at least one analyte present in a biological sample can be retained over a period of time, e.g., regardless of whether the at least one analyte is active or inactive, or intact or non-intact.

As used herein, the term an “off-the-shelf,” component of a device described herein, e.g., a capillary, can refer to a component and/or a portion of a device described herein that was not designed or made to order but, e.g., taken from existing stock or supplies.

As used herein, the term “reference sample,” can refer, in some embodiments, to the presence, quantity, and/or activity of an analyte in a biological sample as measured, e.g., immediately after the biological sample is obtained, e.g., from a subject. In some embodiments, a reference sample can refer to the presence, quantity, and/or activity of an analyte in a biological sample as measured before and/or after the biological sample is mixed or entrapped within a silk fibroin composition by use of a silk-based biological sample collection device as described herein. In some embodiments, a reference sample can refer to the presence, quantity, and/or activity of an analyte in a control sample, e.g., a control stabilized by use of non-silk-based biological sample collection device.

As used herein, a “subject” means a human or animal. Usually the animal is a vertebrate such as a primate, rodent, domestic animal or game animal. Primates include chimpanzees, cynomologous monkeys, spider monkeys, and macaques (e.g., Rhesus). Rodents include mice, rats, woodchucks, ferrets, rabbits and hamsters. Domestic and game animals include cows, horses, pigs, deer, bison, buffalo, feline species (e.g., domestic cat), canine species (e.g., dog, fox, wolf), avian species (e.g., chicken, emu, ostrich), and fish (e.g., trout, catfish and salmon). In certain embodiments of the aspects described herein, the subject is a mammal (e.g., a primate, e.g., a human). A subject can be male or female. In certain embodiments, the subject is a mammal. The mammal can be a human, non-human primate, mouse, rat, dog, cat, horse, or cow, but are not limited to these examples. In addition, the methods and formulations described herein can be used to treat domesticated animals and/or pets.

As used herein, a “substantially dry” or “substantially dried” composition, formulation, or preparation (e.g., of silk fibroin as described herein) refers to a composition in which there is, e.g., 20% (w/w) or less residual moisture content (RMC). A substantially dry formulation or preparation may, in some cases, be prepared by substantially removing the water from a mixture (e.g., a mixture comprising lyophilized silk and a biological sample as described herein) that has been formulated in a solution or liquid mixture. The removal of the liquid can be accomplished by various means (e.g., by air drying, by desiccant facilitated air drying, by passive evaporation, by evaporation assisted by vacuum or other conditions, and/or by sublimation such as by lyophilization (freeze-drying)). In particular embodiments, the substantially dried compositions described herein comprise 5% to 20% (w/w), or at least 4.6% (w/w) (e.g., 4% to 10%), e.g., residual moisture content. In particular embodiments, the substantially dried compositions described herein comprise 0.5% to 5% (w/w) residual moisture content. In the context of a silk fibroin composition as described herein, e.g., dried, e.g., in a device described herein, the substantially dried silk fibroin composition comprises at least about 10% or more silk fibroin by weight (e.g., at least about 10%, at least about 20%, at least about 30%, at least about 40%, at least about 50%, at least about 60%, at least about 70%, at least about 80%, at least about 90%, at least about 95% or more silk fibroin by weight).

As used herein, the term “viruses” refers to an infectious agent composed of a nucleic acid encapsidated in a protein, e.g., that may be stabilized and/or analyzed using the device, composition, and methods descried herein. Such infectious agents are incapable of autonomous replication (i.e., replication requires the use of the host cell's machinery). Viral genomes can be single-stranded (ss) or double-stranded (ds), RNA or DNA, and can or cannot use reverse transcriptase (RT). Additionally, ssRNA viruses can be either sense (+) or antisense (−). Exemplary viruses include, but are not limited to, dsDNA viruses (e.g., Adenoviruses, Herpesviruses, Poxviruses), ssDNA viruses (e.g., Parvoviruses), dsRNA viruses (e.g., Reo viruses), (+)ssRNA viruses (e.g., Picomaviruses, Toga viruses), (−)ssRNA viruses (e.g., Orthomyxoviruses, Rhabdoviruses), ssRNA-RT viruses, i.e., (+)sense RNA with DNA intermediate in life-cycle (e.g., Retroviruses), and dsDNA-RT viruses (e.g., Hepadnaviruses). In some embodiments, viruses can also include wild-type (natural) viruses, killed viruses, live attenuated viruses, modified viruses, recombinant viruses or any combinations thereof. Other examples of viruses include, but are not limited to, enveloped viruses, respiratory syncytial viruses, non-enveloped viruses, bacteriophages, recombinant viruses, and viral vectors. The term “bacteriophages” as used herein refers to viruses that infect bacteria.

In some embodiments, the viruses described herein may be stabilized, thereby allowing for their measurement and/or identification (e.g., by viral load and/or by viral titer). In some embodiments, an infection and/or exposure to a virus described herein can be determined by measuring and/or identifying antibodies to the virus in a biological sample described herein.

As used herein, the term “enterovirus” refers to a virus within the enterovirus genus of positive-sense single-stranded RNA viruses within the picornavirus family. An enterovirus can be a live wild-type virus, a live attenuated virus, an inactivated virus, a chimeric virus, or a viral vector or viral subunit comprising a peptide or protein derived from an enterovirus capsid or genome. Examples of enteroviruses include, but are not limited to, the polio viruses, coxsackie viruses, rhinoviruses and echo viruses.

As used herein, the term “rotavirus” refers to a virus within the rotavirus genus of double-stranded RNA viruses within the Reoviridae family. A rotavirus can be a live wild-type virus, a live attenuated virus, an inactivated virus, a reassortant or chimeric virus, or a viral vector or viral subunit comprising a peptide or protein derived from an rotavirus capsid or genome.

As used herein, the term “flavivirus” refers to a virus within the flavivirus genus of positive-sense single-stranded RNA viruses within the Flaviviridae family. A flavivirus can be a live wild-type virus, a live attenuated virus, an inactivated virus, a chimeric virus, or a recombinant virus. Examples of flaviviruses include, but are not limited to, yellow fever virus, Japanese encephalitis virus, dengue virus, and Zika virus.

As used herein, the term “measles virus” refers to a virus within the morbillivirus genus of single-stranded, negative-sense, enveloped (non-segmented) RNA viruses within the Paramyxovirus family. A measles virus can be a live wild-type virus, a live attenuated virus, an inactivated virus, a chimeric virus, or a recombinant virus.

As used herein, the term “mumps virus” refers to a virus within the rubulavirus genus of linear, single-stranded, negative-sense RNA viruses within the Paramyxoviridae family. A mumps virus can be a live wild-type virus, a live attenuated virus, an inactivated virus, a chimeric virus, or a recombinant virus.

As used herein, the term “rubella virus” refers to a virus within the rubivirus genus of single-stranded, positive-sense RNA viruses within the Togaviridae family. A rubella virus can be a live wild-type virus, a live attenuated virus, an inactivated virus, a chimeric virus, or a recombinant virus.

As used herein, the term “influenza virus” refers to a negative-sense ssRNA virus within the Orthomyxoviridae family. An influenza virus can be a live wild-type virus, a live attenuated virus, an inactivated virus, a chimeric virus, or a recombinant virus. Examples of influenza viruses include influenza A, influenza B, and influenza C.

As used herein, the term “parasite” refers to an organism that lives in or on another organism (e.g., its host) and benefits, e.g., by deriving nutrients at the host's expense. In some embodiments, the parasites described herein may be stabilized and/or analyzed using the device, composition, and methods descried herein, thereby allowing for their measurement and/or identification using art known laboratory assays and instrumentation. In some embodiments, a parasitic infection and/or exposure can be determined, e.g., by measuring and/or identifying a parasite derived analyte from a biological sample described herein (e.g., a stool sample). Exemplary parasites include, but are not limited to, intestinal parasites (e.g., Giardia species) and parasites associate with recreational water-related disease outbreaks (e.g., cryptosporidium), and Entamoeba histolytica. Additional parasites, parasite-related diseases, and/or risk factors for parasite infection include, but are not limited to, Acanthamoeba Infection, Acanthamoeba Keratitis Infection, African Sleeping Sickness (African trypanosomiasis), Alveolar Echinococcosis (Echinococcosis, Hydatid Disease), Amebiasis (Entamoeba histolytica Infection), American Trypanosomiasis (Chagas Disease), Ancylostomiasis (Hookworm), Angiostrongyliasis (Angiostrongylus Infection), Anisakiasis (Anisakis Infection, Pseudoterranova Infection), Ascariasis (Ascaris Infection, Intestinal Roundworms), Babesiosis (Babesia Infection), Balantidiasis (Balantidium Infection), Balamuthia Baylisascariasis (Baylisascaris Infection, Raccoon Roundworm), Bed Bugs, Bilharzia (Schistosomiasis) Blastocystis hominis Infection, Body Lice Infestation (Pediculosis), Capillariasis (Capillaria Infection), Cercarial Dermatitis (Swimmer's Itch), Chagas Disease (American Trypanosomiasis), Chilomastix mesnili Infection (Nonpathogenic Intestinal Protozoa), Clonorchiasis (Clonorchis Infection) CLM (Cutaneous Larva Migrans, Ancylostomiasis, Hookworm), “Crabs” (Pubic Lice), Cryptosporidiosis (Cryptosporidium Infection), Cutaneous Larva Migrans (CLM, Ancylostomiasis, Hookworm), Cyclosporiasis (Cyclospora Infection), Cysticercosis (Neurocysticercosis), Cystoisospora Infection (Cystoisosporiasis) formerly Isospora Infection, Dientamoeba fragilis Infection, Diphyllobothriasis (Diphyllobothrium Infection), Dipylidium caninum Infection (dog or cat tapeworm infection), Dirofilariasis (Dirofilaria Infection), DPDx, Dracunculiasis (Guinea Worm Disease), Drinking water, Dog tapeworm (Dipylidium caninum Infection), Echinococcosis (Cystic, Alveolar Hydatid Disease), Elephantiasis (Filariasis, Lymphatic Filariasis), Endolimax nana Infection (Nonpathogenic Intestinal Protozoa), Entamoeba coli Infection (Nonpathogenic Intestinal Protozoa), Entamoeba dispar Infection (Nonpathogenic Intestinal Protozoa), Entamoeba hartmanni Infection (Nonpathogenic Intestinal Protozoa), Entamoeba, histolytica Infection (Amebiasis), Entamoeba polecki, Enterobiasis (Pinworm Infection), Fascioliasis (Fasciola Infection), Fasciolopsiasis (Fasciolopsis Infection), Filariasis (Lymphatic Filariasis, Elephantiasis), Foodborne Diseases, Giardiasis (Giardia Infection), Gnathostomiasis (Gnathostoma Infection), Guinea Worm Disease (Dracunculiasis), Head Lice Infestation (Pediculosis), Heterophyiasis (Heterophyes Infection), Hookworm Infection, Human, Hookworm Infection, Zoonotic (Ancylostomiasis, Cutaneous Larva Migrans), Hydatid Disease (Cystic, Alveolar Echinococcosis), Hymenolepiasis (Hymenolepis Infection), Intestinal Roundworms (Ascariasis, Ascaris Infection), Iodamoeba buetschlii Infection (Nonpathogenic Intestinal Protozoa), Isospora Infection (see Cystoisospora Infection), Kala-azar (Leishmaniasis, Leishmania Infection), Keratitis (Acanthamoeba Infection), Leishmaniasis (Kala-azar, Leishmania Infection), Lice Infestation (Body, Head, or Pubic Lice, Pediculosis, Pthiriasis), Liver Flukes (Clonorchiasis, Opisthorchiasis, Fascioliasis), Loiasis (Loa loa Infection), Lymphatic filariasis (Filariasis, Elephantiasis), Malaria (Plasmodium Infection), Microsporidiosis (Microsporidia Infection), Mite Infestation (Scabies), Myiasis, Naegleria Infection, Neurocysticercosis (Cysticercosis), Neglected Parasitic Infections in the U.S., Neglected Tropical Diseases, Nonpathogenic (Harmless) Intestinal Protozoa, Ocular Larva Migrans (Toxocariasis, Toxocara Infection, Visceral Larva Migrans), Onchocerciasis (River Blindness), Opisthorchiasis (Opisthorchis Infection), Paragonimiasis (Paragonimus Infection), Pediculosis (Head or Body Lice Infestation), Pthiriasis (Pubic Lice Infestation), Pinworm Infection (Enterobiasis), Plasmodium Infection (Malaria), Pneumocystis jirovecii Pneumonia, Pseudoterranova Infection (Anisakiasis, Anisakis Infection), Pubic Lice Infestation (“Crabs,” Pthiriasis), Raccoon Roundworm Infection (Baylisascariasis, Baylisascaris Infection), Recreational water, River Blindness (Onchocerciasis), Sappinia, Sarcocystosis (Sarcocystosis Infection), Scabies, Schistosomiasis (Bilharzia), Sleeping Sickness (Trypanosomiasis, African; African Sleeping Sickness), Soil-transmitted Helminths, Strongyloidiasis (Strongyloides Infection), Swimmer's Itch (Cercarial Dermatitis), Swimming Pools, Taeniasis (Taenia Infection, Tapeworm Infection), Tapeworm Infection (Taeniasis, Taenia Infection), Toxocariasis (Toxocara Infection, Ocular Larva Migrans, Visceral Larva Migrans), Toxoplasmosis (Toxoplasma Infection), Trichinellosis (Trichinosis), Trichinosis (Trichinellosis), Trichomoniasis (Trichomonas Infection), Trichuriasis (Whipworm Infection, Trichuris Infection), Trypanosomiasis, African (African Sleeping Sickness, Sleeping Sickness), Trypanosomiasis, American (Chagas Disease), Visceral Larva Migrans (Toxocariasis, Toxocara Infection, Ocular Larva Migrans), Waterborne Diseases, Whipworm Infection (Trichuriasis, Trichuris Infection), Zoonotic Diseases (Diseases spread from animals to people), and/or Zoonotic Hookworm Infection (Ancylostomiasis, Cutaneous Larva Migrans).

As used herein, the term a “polymer” refers to a molecule or macromolecule composed of two or more repeated subunits. In some embodiments, the polymer is composed of the same subunits, also referred to herein as a “homopolymer.” In other embodiments, the polymer is composed of different subunits, also referred to herein as a “heteropolymer.” In some embodiments, the polymer, e.g., a protein polymer, comprises a peptide or polypeptide, e.g., a silk fibroin protein, a gelatin protein, an albumin protein, an elastin protein, a collagen protein, a heparin protein, and/or a cellulose protein. In some embodiments, the polymer comprises polyethylene glycol (PEG), polyethylene oxide (PEO), hyaluronic acid, carboxymethylcellulose (CMC), polyvinylpyrrolidone (PVP), and/or polyvinyl alcohol (PVA). In some embodiments, the polymer can be a natural or a synthetic polymer.

As used herein, the term “total solids” refers to a measure of the suspended and/or dissolved solids in a liquid composition, e.g., a composition, mixture, and/or biological sample as described herein. In some embodiments, total solids can be measured by weighing the amount of solids present in a known volume of a liquid composition, e.g., a composition, mixture, and/or biological sample as described herein. In some embodiments, the amount of total solids in a liquid composition, e.g., a composition, mixture, and/or biological sample as described herein, can be optimized and/or adjusted to stabilize, e.g., the structure, integrity, configuration, function, and/or activity of a biological sample, or at least one fraction, component, and/or analyte thereof. In some embodiments, the amount of total solids in a liquid composition, e.g., a composition, mixture, and/or biological sample as described herein, can be optimized and/or adjusted to increase the recovery, e.g., after reconstitution, of a biological sample, or at least one fraction, component, and/or analyte thereof.

The patent, scientific and technical literature referred to herein establish knowledge that was available to those skilled in the art at the time of filing. The entire disclosures of the issued U.S. patents, published and pending patent applications, and other publications that are cited herein are hereby incorporated by reference to the same extent as if each was specifically and individually indicated to be incorporated by reference. In the case of any inconsistencies, the present disclosure will prevail.

### I. Polymer-Based Biological Sample Collection, Separation, and Recovery Devices

Several models are contemplated. One model incorporates the use of a polymer-infused membrane, e.g., silk-infused membrane, e.g., paper, in which the polymer is deposited into paper by coating, printing, dipping, spraying, or similar processes, and then dried. The biological sample reconstitutes the polymer, e.g., silk, and re-dries on the paper, and is eluted in the lab. In certain embodiments, the polymer, e.g., silk, can be deposited on other types of substrates, such as nylon, polypropylene, polyester, rayon, cellulose, cellulose acetate, mixed cellulose ester, glass microfiber filters, cotton, quartz microfiber, polytetrafluoroethylene, polyvinylidene fluoride, and the like. In some embodiments, the substrate can be chemically treated to assist sample retention, mobility, test preparation, or increase longevity.

Another model includes a polymer composition, e.g., a polymer matrix, such as a silk fibroin composition or matrix, contained in a collection device that is configured to receive, dry and support the biological sample for transport and/or storage. With this model, the device is particularly configured to precisely meter, dissolve, dry, solubilize, and recover the biological sample. Specifically, the polymer, e.g., a lyophilized silk fibroin composition as described herein, can be in a collection tube, channel or well in which the biological sample dissolves the polymer, e.g., the silk. A capillary tube is used to meter volume upstream from the polymer matrix, e.g., silk matrix, where the biological sample is deposited on the polymer matrix, e.g., silk matrix. The device includes desiccant to facilitate air-drying of the biological sample. Such a device can be further configured to separate a biological sample, e.g., a blood sample into red blood and plasma. A filter membrane can be employed to separate, e.g., plasma from the whole blood. The membrane can be fabricated from any suitable material, such as plastic, rubber or silicone. The device can be further configured to remove the biological sample within a lab environment by means of lock fittings and threaded parts. In one embodiment, the device can employ a vertical orientation to use gravity to aid in sample flow within the device. In another embodiment, the device can be horizontally oriented to enable more controlled metering with a pressure device, e.g., a plunger, used to generate a positive pressure to move the biological sample through the device.

As mentioned above, in one embodiment, the device includes a capillary tube or channel to draw specimen into the device via capillary action. The capillary tube or channel can include different dimensions to accommodate and meter desired volumes. A fill indicator can be provided on the capillary tube, e.g., markings provided on the tube or device, to enable the user to determine when enough specimen is collected. In another embodiment, a coating as described herein, e.g., an anticoagulant, can be coated on an interior of the tube.

In a certain embodiment, polymer, e.g., silk is lyophilized, in the capillary tube or channel in which the polymer, e.g., silk, is dried inside the tube or channel using lyophilization. Alternatively, polymer, e.g., silk, is lyophilized in a sample collection area, such as a well, trough, or the like. Optional features may include grooves or pillars that can be added into tube or channel to enhance mixing of silk with specimen.

Drying can include air drying the sample prior to analysis. In one embodiment, samples can be dried in ambient conditions or with the aid of desiccant as described herein to increase the drying rate. Desiccant can be provided in the device, and exemplary desiccants include, but are not limited to, a molecular sieve, silica gel, montmorillonite clay, calcium oxide, calcium sulfate, and mixtures thereof.

When employing the polymer-infused (e.g., silk-infused) membrane, the sample can be punched, similar to dried blood spots, prior to elution of analytes. When employing the collection device having a detachable component in the shape of a well, trough or channel, the detachable component can be coated with a surfactant or a hydrophilic coating or material to aid in the spreading of liquid on a surface of the component. For analysis, the biological sample can be reconstituted in solution, e.g., in water, a buffer, or an organic solvent. Elution time can range anywhere from a short time, e.g., about 5 minutes (e.g., about 5 minutes, about 10 minutes, about 15 minutes, about 20 minutes, about 25 minutes, about 30 minutes, about 35 minutes, about 40 minutes, about 45 minutes, about 50 minutes, about 55 minutes, or about 60 minutes), to a long time, e.g., about 2 hours to overnight (e.g., about 2 hours, about 3 hours, about 4 hours, about 5 hours, about 6 hours, about 7 hours, about 8 hours, about 9 hours, about 10 hours, about 11 hours, about 12 hours or longer). In some embodiments, the sample can then be transferred into a separate tube or fit into an instrument for analysis. In some embodiments, the device (e.g., a portion of a device described herein, such as a drying and recovery portion) is compatible with laboratory assays and/or instrumentation, e.g., such that use of such assays and/or instruments would not require transfer of the polymer composition (e.g., a substantially dried and/or reconstituted polymer fibroin composition) out of the device. In some embodiments, device (e.g., a portion of a device described herein, such as a drying and recovery portion and/or a recovery portion) can be fit into an instrument for analysis.

Optionally, the collection device can be configured to fractionalize the biological sample. A single device can be provided for the sole purpose of plasma isolation, or accessories can be retrofit into the device to enable separation of plasma prior to drying. Lateral flow (e.g., Whatman LF1, Ahlstrom CytoSep®, etc.) or cross flow (e.g., Pall Vivid™) separation technologies can be employed. For cross flow products, an additional collection membrane can be used to wick the plasma from the separation membrane. Polymer, e.g., silk can also be impregnated into the plasma separation or collection membranes.

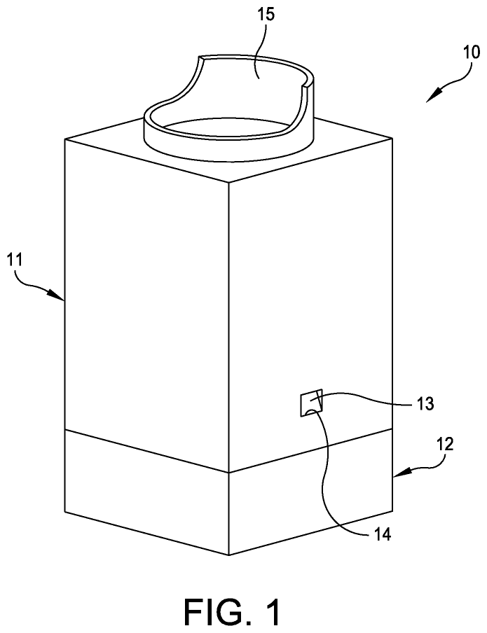

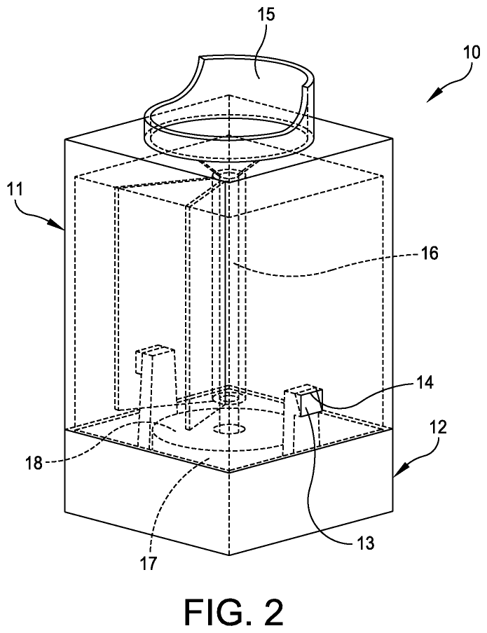

Referring to the drawings, exemplary devices are shown and described herein. In one embodiment, shown in FIGS. 1 and 2, a collection device is generally indicated at 10. As shown, the collection device 10 is vertically oriented, having a sample collection portion, generally indicated at 11, which can be transparent to aid in the metering of a biological sample, e.g., blood, into the device. The sample collection device 10 further includes a removable base, generally indicated at 12, which is configured to snap fit to the sample collection portion when releasably securing the sample collection portion 11 to the removable base. As shown, the sample collection portion 11 and the removable base 12 are generally square in construction; however, the structure of these component parts may embody any suitable shape and/or size. For example, the sample collection portion and the removable base can be cylindrical in construction.

As best shown in FIG. 2, the removable base 12 can include a pair of snap fit members, each indicated at 13, which are configured to be received within openings, each indicated at 14, formed in the sample collection portion 11. To remove the sample collection portion 11 from the removable base 12, the snap fit members 13 are moved inboard to release the members from their respective openings 14 thereby enabling the lifting of the sample collection portion from the removable base.

The sample collection portion 11 includes a funnel-shaped collector 15 having a scoop to aid in sample collection. The collector 15 is configured to direct the sample to an opening that contains a capillary tube 16 to direct the sample through the device. In a certain embodiment, the capillary tube 16 is configured to meter the sample through the device, e.g., 50 μL to 400 μL of, e.g., blood, depending on the size of the capillary tube. In one embodiment, silk is lyophilized in the capillary tube 16 in which the silk is dried inside the tube using lyophilization. In another embodiment, polymer, e.g., silk, can be lyophilized in a sample collection area, such as a well, trough, or the like, associated with the capillary tube 16. Optional features may include grooves or pillars that can be added into tube to enhance mixing of polymer, e.g., silk, with specimen. The sample collection portion 11 further includes a ribbed portion that separates desiccant contained within the collection device from visual metering. The removable base 12 includes a well 17 configured to contain the biological sample for easy elution. The removable base 12 can also contain the desiccant described above. The sample collection device 11 further includes a porous membrane 18, e.g., fabricated from mesh material, that is seated on the well 17. The porous membrane 18 also separates the desiccant from the well 17. The sample collection device 10 may include external packaging to cover the working components of the sample collection portion 11. The sample collection portion 11, the removable base 12 and the external packaging can be fabricated from molded plastic, such as polypropylene, polystyrene, polyethylene terephthalate (PET), polydimethylsiloxane (PDMS), poly(methyl methacrylate) (PMMA), poly(tetrafluoroethylene) (PTFE) and/or from paper. The membrane 18, which can be in the form of a pouch, and the desiccant can be purchased as off-the-shelf components.

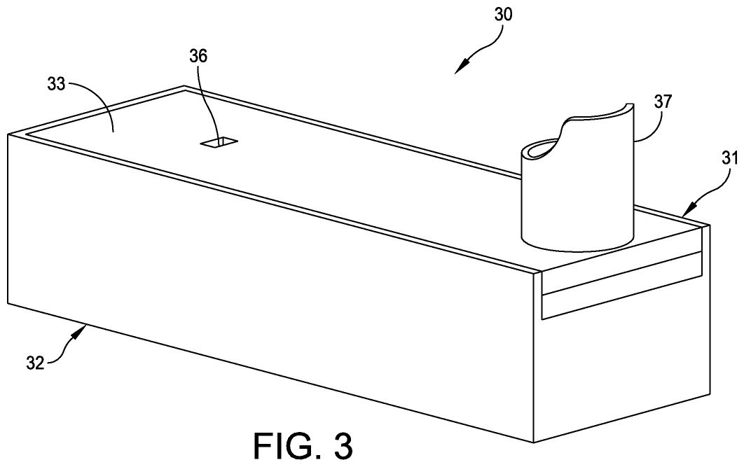

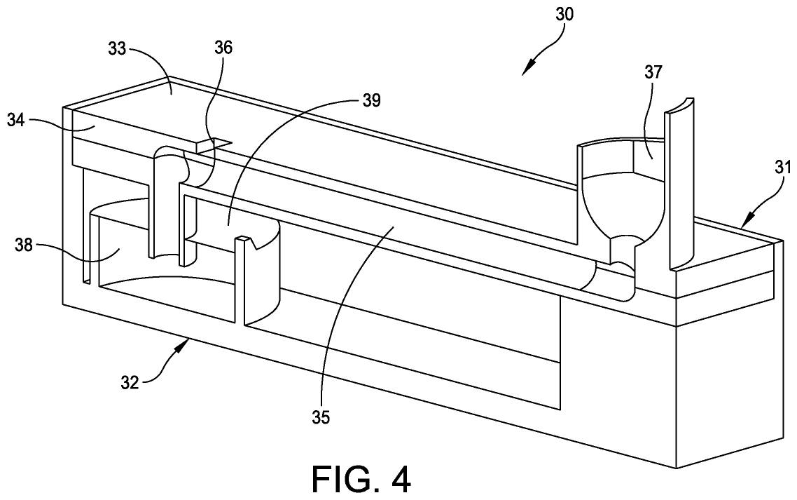

In another embodiment, shown in FIGS. 3-5, a collection device is generally indicated at 30. As shown, the collection device 30 is horizontally oriented, having a sample collection portion, generally indicated at 31, and a base portion, generally indicated at 32, which is configured to be secured to the sample collection portion. As shown, the sample collection portion 31 includes a top part 33 and a bottom part 34 that is disposed under the top part and is configured to support and position a capillary tube 35. The top part 33 of the sample collection portion 31 is configured to include a window 36 to enable a user to monitor when collection of a sample, e.g., blood, is complete. In one embodiment, once fully assembled, the sample collection portion 31 can be permanently affixed, e.g., by adhesive, to the base portion 32 to achieve the configuration shown in FIG. 3. In another embodiment, the sample collection portion 31 and the base portion 32 can be configured to be releasably secured to one another in a suitable manner.

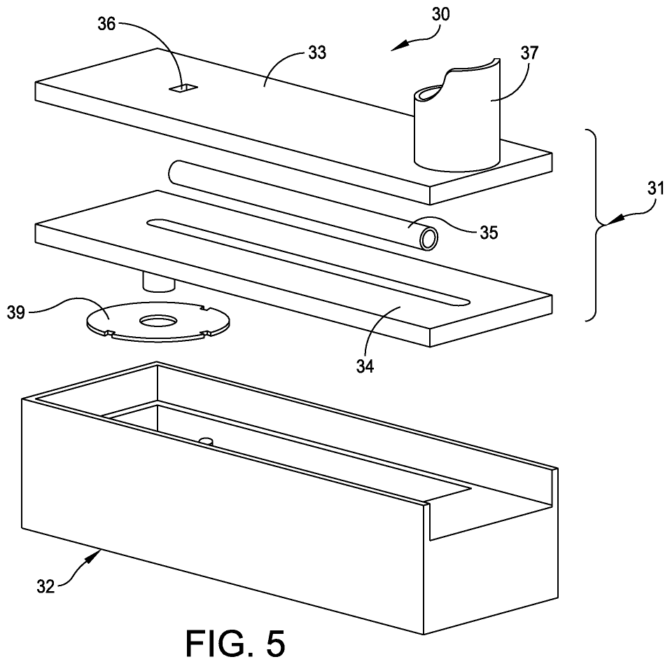

In one embodiment best shown in FIG. 5, the sample collection portion 31 includes the top part 33 and the bottom part 34 that are assembled together to create a channel to hold or otherwise create the capillary tube 35. In a certain embodiment, the capillary tube 35 is configured to include silk and/or an anticoagulant coating. In one embodiment, polymer, e.g., silk, is lyophilized in the capillary tube 35 in which the polymer, e.g., silk, is dried inside the tube using, e.g., lyophilization. In another embodiment, polymer, e.g., silk, can be lyophilized in a sample collection area, such as a well, trough, or the like, associated with the capillary tube 35. Optional features may include grooves or pillars that can be added into tube to enhance mixing of polymer, e.g., silk, with specimen. The top part 33 of the sample collection portion 31 includes a funnel-shaped collector 37 having a scoop to aid in sample collection. The collector 37 is configured to direct the sample to an opening that is in fluid communication with the capillary tube 35. The base portion 32 includes a well 38 configured to contain the biological sample for easy elution. The base portion 32 can also contain the desiccant described above in part of the base portion next to the well 38. The sample collection device 30 further includes a porous membrane 39, e.g., fabricated from mesh material, provided in the well 38. In one embodiment the membrane 39 can be configured to separate the desiccant from the well 38. A lid may also be fabricated out of desiccant.

Although not shown, a piston or plunger can be provided to pressurize fluid within the capillary tube 35 to enhance the flow of the biological sample to the well 38. The arrangement is such that pressurized air in fluid communication in the capillary tube 35 forces the biological sample through the capillary tube. The sample collection portion 31 and the base portion 32 can be fabricated from molded plastic. The capillary tube 35, the membrane 39, which can be in the form of a pouch, and the desiccant can be purchased as off-the-shelf components.

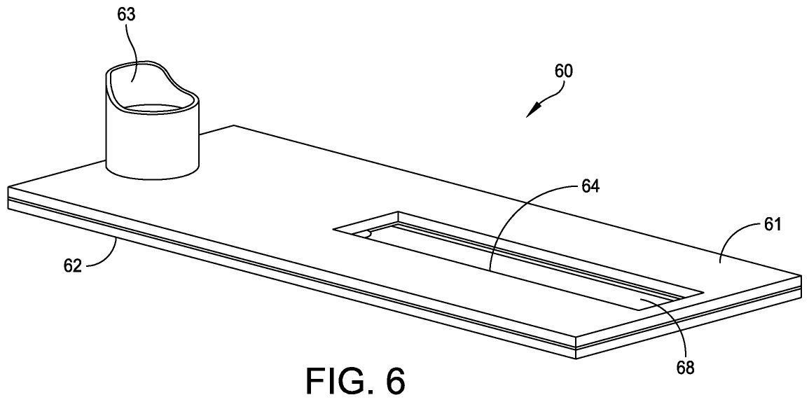

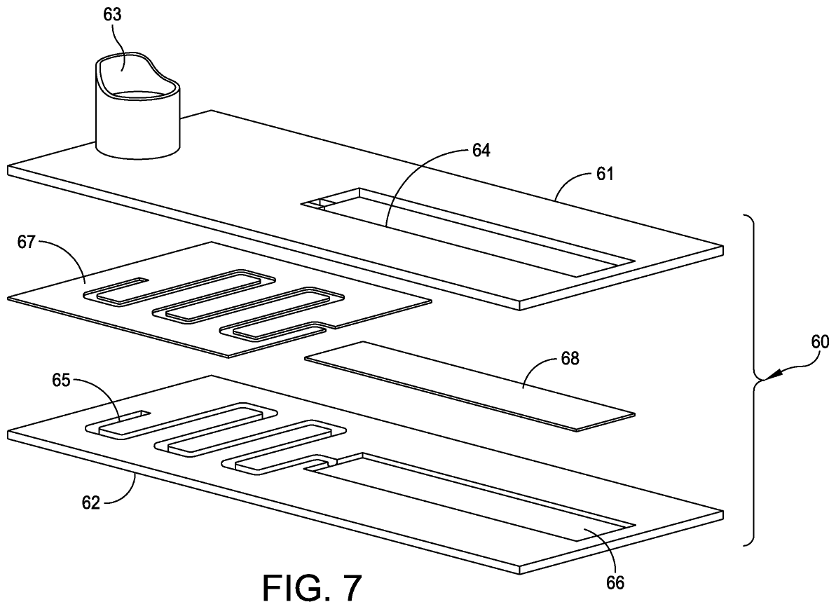

In another embodiment, shown in FIGS. 6 and 7, a collection device is generally indicated at 60. As shown, the collection device 60 embodies a plasma separation card that includes a top layer portion 61 and a bottom layer portion 62 that together form the shape of the plasma separation card. The top layer portion 61 includes a funneled-shaped scoop inlet 63 to direct a biological sample into the plasma separation card 60. For example, blood from a finger prick wicks into the plasma separation card 60 via capillary action. The top layer portion 61 further has a window 64 formed therein to evaluate when the card is fully loaded with a biological sample. The bottom layer portion 62 includes a pathway 65 and a collection area 66 formed therein to assist the flow of blood from the pathway to the collection area. In one embodiment, the bottom layer portion 62 can include a coating, such as an anticoagulant coating or a surfactant coating, to enhance wetting. The bottom layer portion 62 can also contain a sample collection area, such as a well, trough, or the like, associated with the bottom layer portion 62. Optional features may include grooves or pillars that can be added into bottom layer portion to enhance mixing of polymer, e.g., silk, with specimen. In another embodiment, the bottom layer 62 can include grooves to aid in mixing, and can be press-fit or glued with the top layer 61 to create a seal.

The plasma separation card 60 further includes a tape layer 67 to enhance sealing. In another embodiment the tape layer 67 can be ultrasonically welded to the bottom layer 62. The plasma separation card 60 further includes a lateral flow plasma separation membrane 68, which in one embodiment may have paper that can be removed from the plasma separation card 60 or punched within the card for analysis of sample. In one embodiment, the plasma separation card 60 can be enclosed in a transport vessel with sealed desiccant to enhance drying. The top layer 61 and the bottom layer 62 can be fabricated from molded plastic. The tape layer 67 and the separation membrane 68 can be purchased as off-the-shelf components. In some embodiments, a silk fibroin composition is impregnated into the plasma separation paper 68.

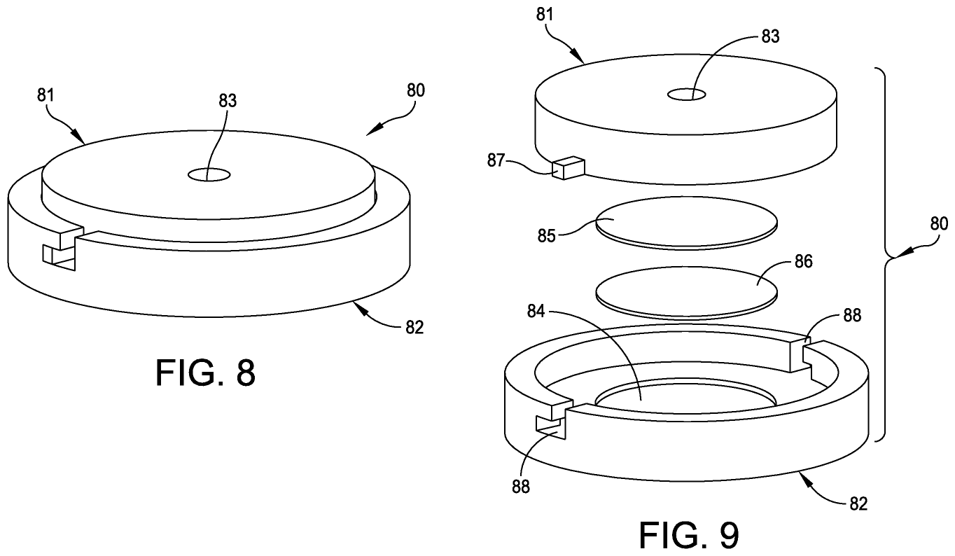

In another embodiment, shown in FIGS. 8 and 9, a sample separation accessory is generally indicated at 80. As shown, the sample separation accessory 80 is configured to separate plasma from whole blood, and includes a disc-shaped top portion, generally indicated at 81, and a disc-shaped base portion, generally indicated at 82, which are configured to be releasably secured to one another. Although disc-shaped, the top portion 81 and the bottom portion 82 can embody other shapes and sizes. The disc-shaped top portion 81 of the sample separation accessory can be configured to be retrofitted into an end of the capillary tube 16 of the collection device 10 described above, for example, via opening 83. The disc-shaped base portion 82 includes a well 84 configured to receive a vertical flow plasma separation membrane 85 and a collection layer 86 configured to passively receive plasma through wicking. Collection layer 86 can be a polymer-infused (e.g. paper containing a polymer, e.g., a lyophilized silk fibroin composition) or simply a stand-alone polymer composition, such as a lyophilized silk fibroin composition. Collection layer 86 can also be entirely comprised of a polymer composition as described herein, e.g., a silk fibroin composition. Pressure from a plunger may be used to displace the plasma from the membrane to the polymer composition, e.g., the lyophilized silk fibroin composition.

As shown, the disc-shaped top portion 81 and the disc-shaped base portion 82 are configured with a locking mechanism to releasably secure the disc-shaped top portion to the disc-shaped base portion. In one embodiment, the locking mechanism can include a bayonet connector having cylindrical male side with one or more radial pins 87 provided on the disc-shaped top portion 81, and a female receptor with matching L-shaped slots, each indicated at 88, provided on the disc-shaped bottom portion 82 to keep the two parts locked together when assembled. Each slot 88 is L-shaped to receive its respective pin 87 as the pin slides into a vertical arm of the L-shaped slot and is moved across the horizontal arm of the L-shaped slot. The disc-shaped top portion 81 and the disc-shaped base portion 82 can be fabricated from molded plastic. The membrane 85 and the collection layer 86 can be purchased as off-the-shelf components.

### Lateral Flow Devices

With the paper and lateral flow approach, methods for adding a polymer as described herein, e.g., silk, gelatin, or a combination thereof, to paper may be employed. For example, in some embodiments, the polymer, e.g., silk, may be added by spray, stripe, dot, and dip processes. Methods of drying polymer formulation on paper may include in-line freezing then lyophilization, in-line lyophilization, or air drying. When applying the polymer, e.g., silk, to paper, patterns may be used to ensure homogenous mixing between the polymer, e.g., silk and the sample. Such patterns include even distribution, stripes along flow, stripes perpendicular to flow, array of dots, and adding in only part of the paper (beginning, middle, or end).

In some embodiments, a device with various porous materials in contact with each other for best polymer mixing and plasma separation is implemented. For example, the material used in the device can be configured to perform plasma separation and another material used in the device can be configured to enable mixing with silk and dry down. In still other embodiments, a third material can be used for dry-down.

In some embodiments, the addition of the polymers, e.g., silk, to lateral plasma separation membrane yields a larger plasma sample area from which to draw sample.

In some embodiments, a cassette is provided to enclose lateral flow plasma separation membrane that provides a guide to where sample should be deposited, prevents finger from touching membrane, and provides a clear window indicating sufficient sample.

In some embodiments, blood is collected in a capillary or vial with lyophilized polymer, e.g., silk, formulation and anticoagulant and dispensed onto a lateral plasma separation membrane. This configuration provides precise metering of the sample, and homogenous mixing with silk and anticoagulant before deposit. This approach also includes a benefit of larger plasma sample area from which to draw sample.

Exemplary lateral flow devices are disclosed herein below in the context of silk. It shall be understood that any other polymer as described herein can be used in the devices, alone or in combination with silk.

Lateral Flow with Silk Collection Device

Referring to FIG. 10, a device 100 includes a bound glass fiber filter (such as LF1) (device 100A) or a plasma separation membrane material (device 100B), each of which has a silk formulation applied. The bound glass fiber filter includes a plasma separation membrane applied to a backing card and hydrophobic backer. Silk is applied to absorbent material. In some embodiments, patterns may be employed by spraying, dipping, or depositing lines or dots of the silk formulation to various regions of the membrane. Additional embodiments may include using multiple separate materials to separate plasma, mix with silk, then dry down (not necessarily in that order).

In some embodiments, a blot (e.g., 200 uL) of whole blood is applied to the device. The blood separates into plasma, with the plasma region cut away. The weighed plasma region is punched (e.g., 5 mm samples) and transferred to a well, where the sample is eluted for one-hour at RT on an orbital shaker.

Once processed, the samples are analyzed. Silk formulations A and B showing that the formulated whole blood increases the plasma yield on glass fiber separation membranes.

Lateral Flow with Silk in Cassette Collection Device

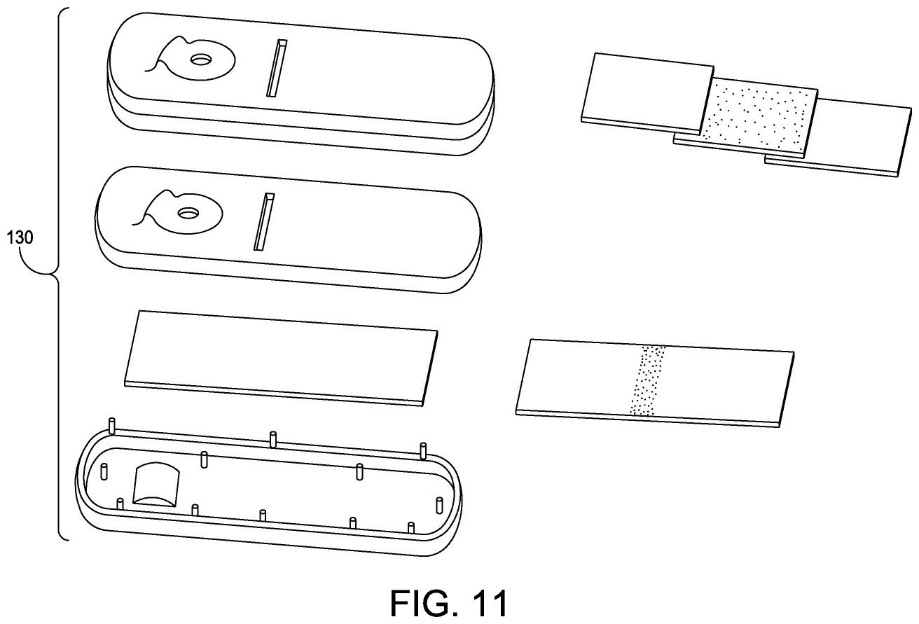

Referring to FIG. 11, a device 130 includes a cassette that houses the plasma separation membrane. In some embodiments, the plasma separation membrane described above is housed in a plastic housing of the cassette, which provides features to increase usability and increase likelihood the user collects a viable sample. The cassette includes a finger wiper feature, which enables a user to frequently transfer blood from fingertip to device. A port directs a blood sample onto the correct area of the membrane. The cassette further includes a window to enable the user to visually determine whether he or she has deposited a sufficient sample. The plastic housing includes vent holes on sides and bottom and may include features to hold desiccant to enhance drying.

### Finger Hug (Lateral) Collection Device

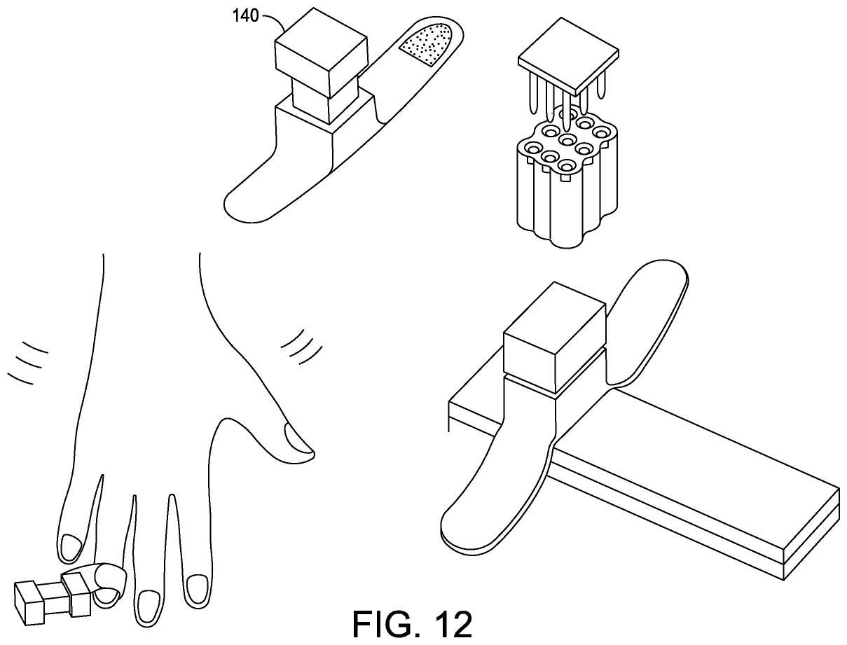

Referring to FIG. 12, a sample collection device 140 includes a housing attached to a strap. The housing supports a plunger array that operates with a capillary array having silk and anticoagulant applied to the capillaries. The arrangement is such that by pressing the plunger array a sample is deposited into the device that may include a silk formulated membrane, such as the membranes described herein. The device is configured to mix the sample with anticoagulant, mix with silk, dry in a well, ship and reconstitute the sample. In operation, after lancing a finger, a user attaches a finger-mounted device to the finger to collect blood into an array of capillaries filled with silk and anticoagulant. This enables collection of a precise amount of whole blood while concealing the blood during collection, and allows for hand to hang at the side to induce blood flow. The device is configured to reduce hemolysis due to finger milking by preventing milking at the fingertip through covering the fingertip with the strap, and to move the point of milking away from fingertip and toward the recommended site at the base of the finger. The device further includes an indicator light, which flashes when all capillaries are fill, which may be accomplished by sensing location of blood in the capillaries, or by monitoring color of capillary stoppers that change color when the sample is in contact. In another embodiment, the capillary plungers turn red to indicate the device has collected sufficient sample. The device is then placed on another device that may or may not include plasma separation, mixing with anticoagulant, mixing with silk, and well for dry-down, shipping, and reconstitution. Sample collection device is depressed to cause plungers to expel sample. The device also can be configured to include a finger lancing feature.

### Vertical Flow Devices

Exemplary vertical flow devices are disclosed herein below in the context of silk. It shall be understood that any other polymer as described herein can be used in the devices, alone or in combination with silk.

In some embodiments, an all-in-one device includes sample metering, plasma separation, mixing with silk, mixing with anticoagulant, and drying down.

In some embodiments, a system with two parts includes a sample metering device that also might include anticoagulant and silk mixing and a sample processing device that has plasma separation and drying down and possible silk mixing and anticoagulant mixing. System with two parts enables micro-sampling (measuring a small volume) and then applying it to second device. Two sample types include whole blood and plasma/serum.

In some embodiments, precise sample metering is provided, which is easy for an untrained home user. An absorbent cap is provided to wick away excess sample in a vial, allowing a user to overfill a vial and still yield a precise sample. In one embodiment, a capillary device that is filled completely horizontally as seen in visual inspection, is then tipped to dispense. In another embodiment, a capillary device with indicator light or sound that goes off when it is completely full.

In some embodiments, a device is provided that enables untrained home users to collect a sample with reduced hemolysis. In one embodiment, a capillary device that straps to finger after lancing the finger to prevent milking or squeezing near the fingertip which leads to hemolysis.

In some embodiments, a device is provided that limits user interaction with blood during fingerstick sample collection to reduce fear reactions from home users. In one embodiment, a capillary device straps to finger after lancing the finger and provides electronic indication of completion. The device is configured to cover the user's fingertip during collection preventing the user from seeing blood. In another embodiment, a capillary device straps to finger after lancing the finger and provides color change in capillary stoppers to indicate when collection is finished, thereby minimizing users seeing blood.

Sponge and Vacuum with Tube Collection Device

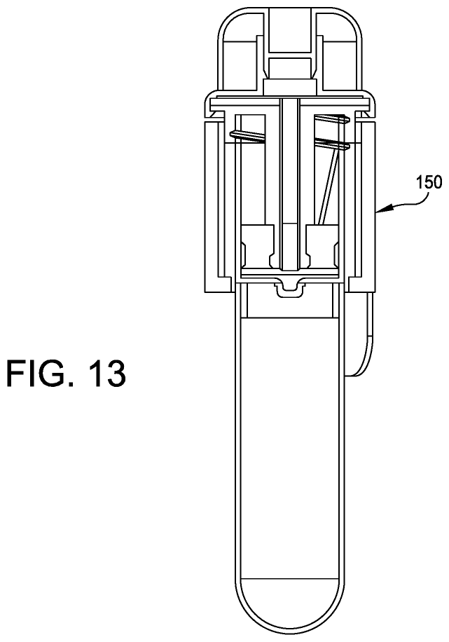

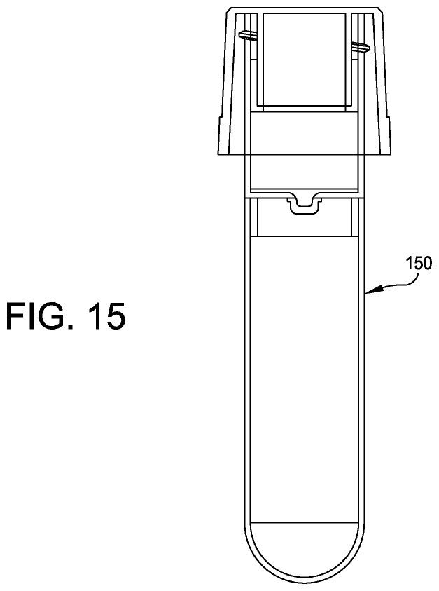

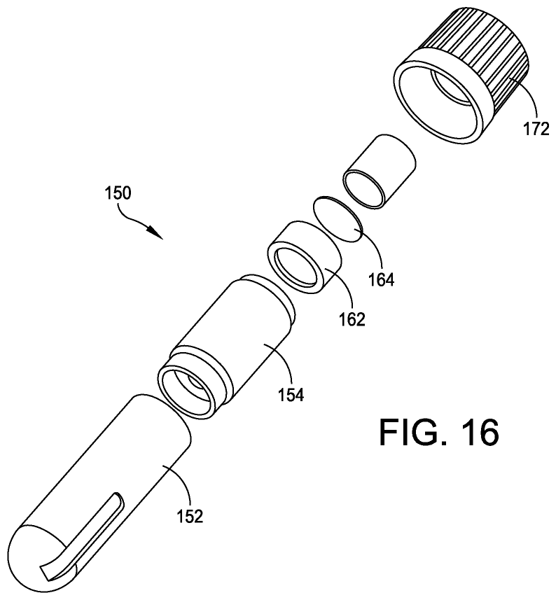

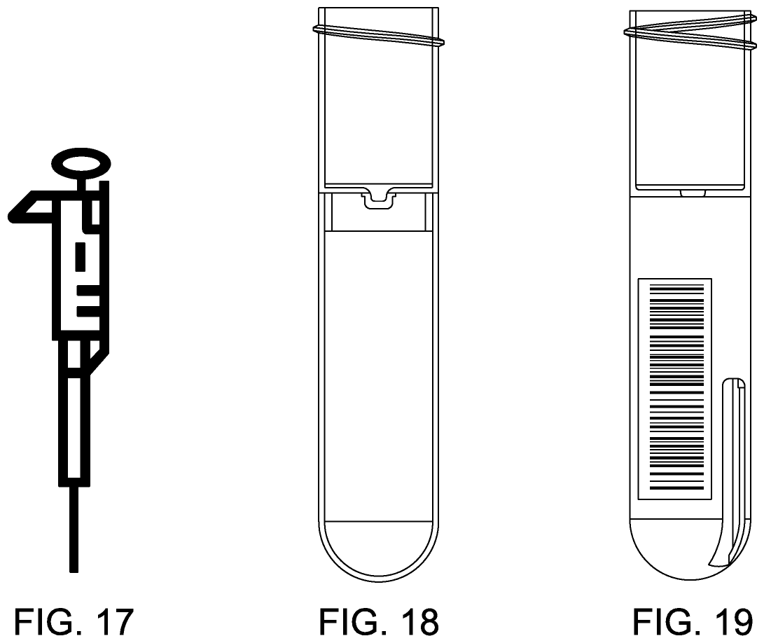

Referring to FIGS. 13-22, a sample collection device 150 includes a tube 152, a well 154, and a thread stop 156. In one embodiment, the tube 152 has a standard outer dimension that fits within an industry-standard tube rack. The tube 152 can be labeled with patient identifying information and/or a barcode, as illustrated in FIG. 19, at a sample collection site and are usable by a clinical lab. The well 154 is positioned in the tube 152, and configured to receive and dry a mixture of a biological sample and a silk fibroin composition as described herein. The sample collection device 150 further includes a sample collection assembly that is supported by the tube 152, well 154 and thread stop 156. As shown, the sample collection assembly includes a plunger 158, a capillary and plunger holder 160, a capillary 162 with lyophilized silk formulation and coated with anticoagulant, a hydrophobidis 164 (e.g., polydimethylsiloxane (PDMS)), a plasma separation membrane 166, a sample collector 168 that is coated with anticoagulant, an absorbent material 170 and an absorbent cap 172, which is secured to the sample collector.

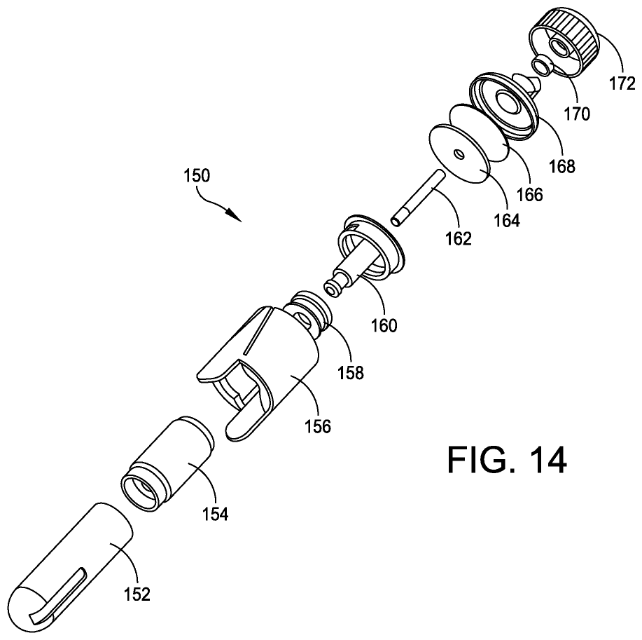

The components of the sample collection device 150 are configured to be assembled in the manner illustrated in FIG. 14. For example, the capillary holder 160 is configured to secure the capillary 162 and the other components of the sample collection assembly to the sample collector 168. The capillary 162 is in fluidic contact with the plasma separation membrane 166, which is positioned in the sample collector 168. The thread stop 156 is configured to be removed to enable the sample collection assembly to be unscrewed from the well 154. The sample collection assembly is configured to receive and mix the biological sample with the silk fibroin composition. The sample collection assembly further is configured to deliver the mixture to the well 154 and to allow a precise amount of the biological sample and the silk fibroin composition to form the mixture. In some embodiments, the sample collector 168 is coated with anticoagulant. In some embodiments, the PDMS is positioned on a surface within the well 154. In some embodiments, the sample collection assembly includes markings or form factor that are aligned with markings or form factor on the well 154 to indicate that the plunger 158 is in a correct position to generate negative pressure in the well.

In a sample collection mode, whole blood is deposited into the sample collector 168 coated with anticoagulant until it is above a line, then capped with the absorbent cap 172, which is configured to be releasably secured to the sample collector. The absorbent cap 172 includes the absorbent material 170 that extends to a precise distance, wicking away excess sample to achieve a precise sample volume. The bottom of the sample collection vessel is the plasma separation membrane 166, eliminating the need for fluidic transfer from the sample collection and plasma separation step.

Using the plasma separation membrane 166 as the bottom of the collection vessel also reduces loss of sample volume and loss of analytes through binding by reducing the number of different vessels and materials with which the sample comes into contact. The plunger 158 is coupled to the capillary holder 160 and the thread stop 156. The user rotates the capillary 162 and the plunger holder 160 in its threads until engaging a detent and/or stopping against the thread stop 156, which moves the plunger linearly, thereby providing negative pressure in the well 154, drawing plasma/serum through the membrane. The result is that the biological sample enters the capillary 162 with lyophilized silk formation and anticoagulant and exits through the plunger 158 into the well 154. In addition, parts of the thread stop 156, sample collector 168, or capillary and plunger holder 160 can incorporate a visual alignment feature that queues the user to align it with a visual indicator on the tube or tube rack, making it easier for untrained users to rotate the correct number of degrees. This plasma separation method allows an untrained user to perform quality plasma separation while minimizing hemolysis and maximizing yield. Once through the plasma separation membrane 166, plasma/serum enters a capillary with lyophilized silk formulation and anticoagulant and exits into the well 154. Capillary enables homogenous mixing of silk formulation with full plasma/serum sample. The user removes the thread stop 156 either through disengaging a snap feature or by peeling off a plastic-like the strip to an orange juice container, enabling sample collection assembly to be unscrewed from the well upwardly with respect to the tube.

In a shipping mode, the user peels a foil seal from inside of the absorbent (e.g., desiccant) cap 172, or removes the absorbent cap from a sealed bag. The absorbent cap 172 is pushed onto well until it engages a stop. The absorbent cap 172 includes the absorbent material 170 (e.g., desiccant material) and a vapor-permeable membrane to allow water vapor to be absorbed. In one embodiment, the tube 152 stays vertical, sample dries in flat dimple well as shown. A tube rack integrated into or separate from the kit packaging can be used to maintain the tube 152 in vertical orientation during plasma separation and drying steps. In another embodiment, a bottom of well is Eppendorf-shaped. The tube 152 is inserted in packaging or a stand by aligning tube orientation feature with a feature on packaging/stand that holds the device at a tilted angle. This enables biological sample to spread out across the side of the device for faster drying. A patient barcode label can be applied at sample collection site to maintain patient-sample match. This can be the same barcode label as used for sample tracking in the clinical lab.

In some embodiments, a dimpled well is provided. A desiccant cap is removed, and a pipette is used to add water to reconstitute the dried film sample to the same original volume. A pipette is also used to mix the sample. The dimpled well is deep enough to enable a pipette tip to access the sample.

In some embodiments, an Eppendorf-shaped well concept is provided. A sample is reconstituted while tilted to ensure pipette water runs over dried sample on side of tube. Then tube is placed vertical so sample is in Eppendorf-shape to provide adequate sample depth for access by a pipette.

In some embodiments, a tube exterior includes standard vacutainer dimensions to fit directly into automated analyzers. The sample stays in the barcode-labeled tube until analysis to maintain patient-sample match. The sample does not require transfer to any other vessel before analysis which reduces loss of sample volume and loss of analytes through binding by reducing the number of different vessels and materials with which the sample comes into contact.

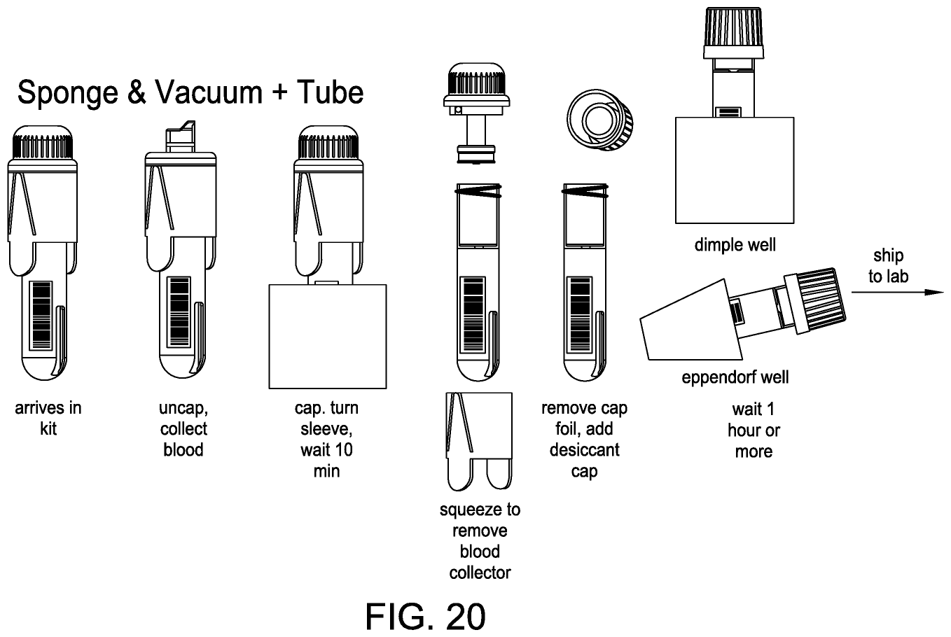

FIG. 20 illustrates a process for collecting a sample. The sample collection device arrives in kit form. Next, the cap of the sample collection device is removed, and a sample is collected by pressing the user's finger against a finger wipe feature. Once collected, the cap is secured to the collector and the sample can rest for a period of time, e.g., ten minutes. The user rotates the capillary and plunger holder, which moves the plunger to draw plasma through the membrane. Next, the user removes thread stop, and enables the sample collection assembly to be unscrewed from well upward. The tube is capped with the desiccant cap. After a period of time, e.g., one hour, the sample collection device can be shipped to a lab for processing.

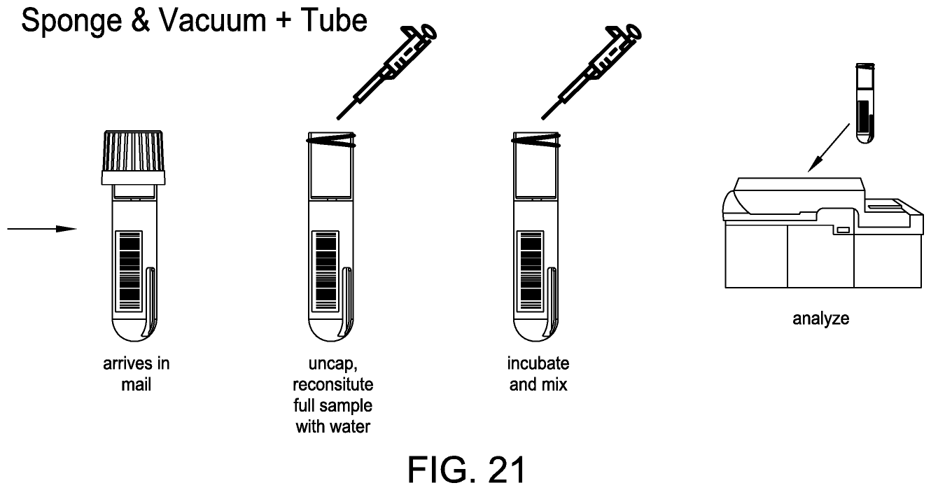

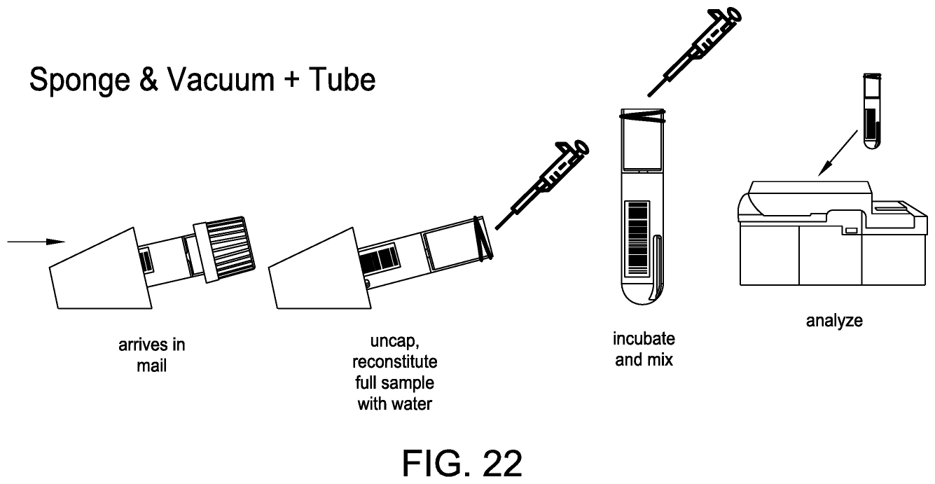

FIG. 21 illustrates a process for analyzing the sample. The sample collection device arrives by mail, and the cap is removed to reveal the sample. The sample is reconstituted with water or other reagents indicated by the target assay and allowed to incubate and mix. Once sufficiently mixed, the sample is analyzed. In another embodiment, FIG. 22 illustrates a process similar to the process shown in FIG. 21, that uses the Eppendorf-shaped well concept. Tube outer diameter and height match standard tube dimensions used in assays and instrumentation known in the art.

### Twist Wand Collection Device

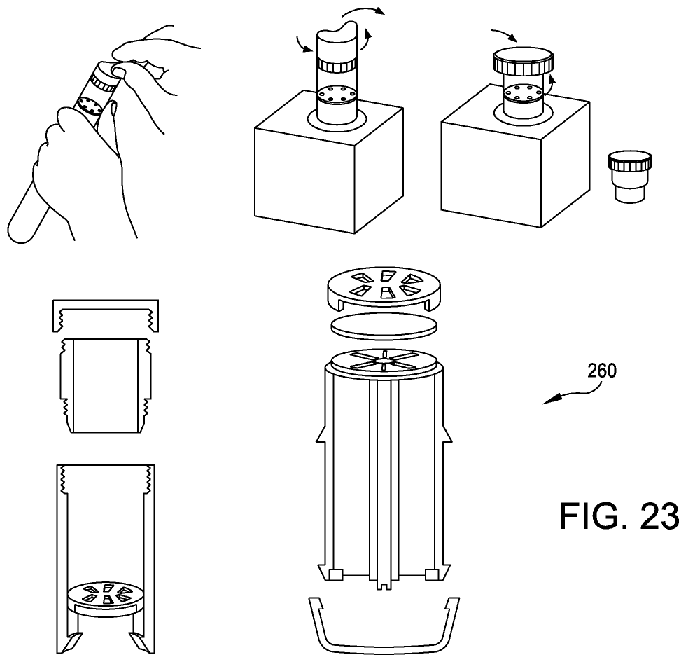

Referring to FIG. 23, a sample collection device 260 includes a container secured to a well. The container houses a membrane, a capillary disc, desiccant, a capillary with silk and a hydrophobic membrane. With help of a raised wiping ridge, blood is deposited into the container or vial until the container is filled above a line. The user twists to close an aperture and separate the top portion of the sample collection container to remove the excess blood. Then the user twists the remaining sample collection vial to align holes allowing blood to pass through to a plasma separation membrane. The user waits for a period of time, e.g., one hour, before mailing. Inside, gravity and a capillary action draws the plasma through a membrane and into the capillary to mix with a silk formulation, then into a well. The well is exposed to desiccant through a vapor-permeable membrane and the sample dries into a film. At the lab, the well is removed and used to reconstitute the sample for analysis.

### Finger Hug and Simple Cup Collection Device

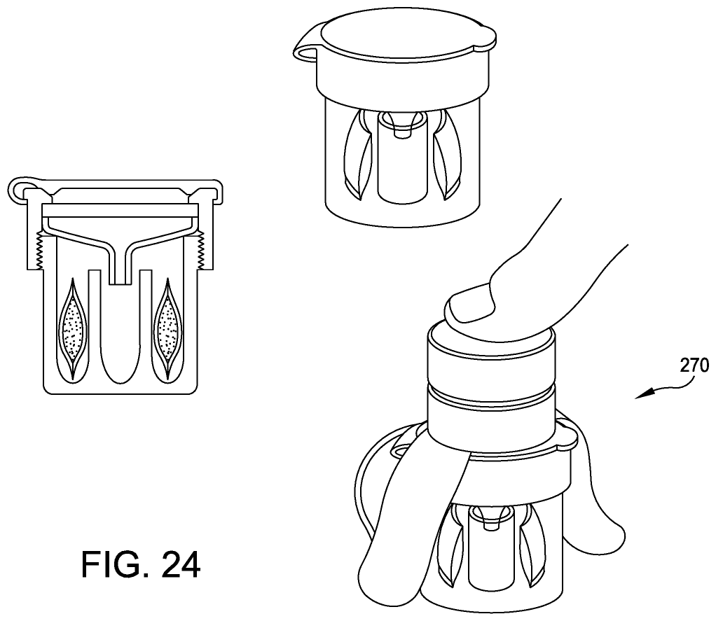

Referring to FIG. 24, a sample collection device 270 includes a container having a flip top. The container houses a membrane, a capillary disfunnel, desiccant sachets and a well for dry-down of the sample. The top is flipped open and whole blood that may or may not be pre-mixed with silk and may or may not be mixed with anticoagulant is deposited onto the plasma separation membrane using a finger hug or another method, and then the lid is closed. Gravity and capillary action help the plasma/serum filter through the membrane and a funnel guides the sample into a well which is open to another chamber with desiccant pouches. In another embodiment the sample passes through a funnel that is coated with anticoagulant and that includes dried silk formulation in the form of a coating, a cake, or pellets. Sample mixes with silk formulation and anticoagulant in the funnel, then falls into a sample well which is open to another chamber with desiccant pouches. The sample dries into a film in the sample well. The lid assembly is unscrewed once it reaches the lab, the membrane and the funnel are removed, and the well is used for sample reconstitution.

### Well Geometry

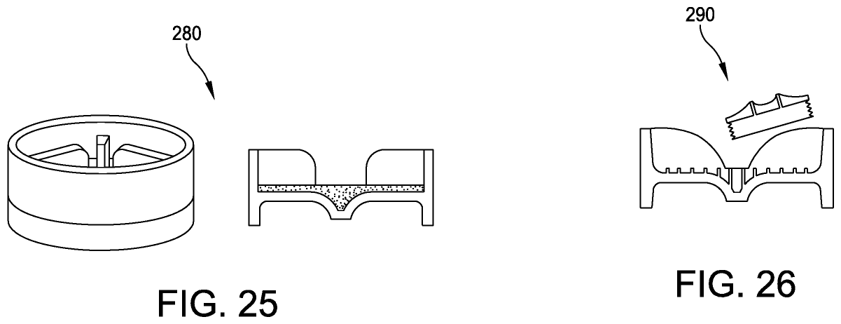

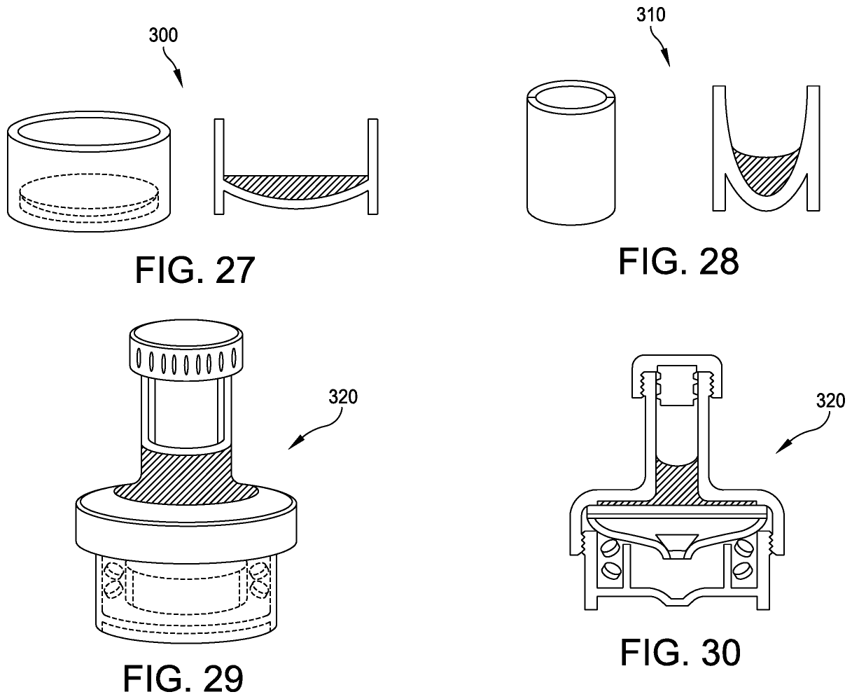

Referring to FIGS. 25-28, various well geometries are illustrated. The well geometry must spread a sample over the largest possible surface area for fast drying, while enabling the lab to add water or other reagents to all of the sample for resuspension. In some embodiments, the well is designed to have a portion deep enough to receive pipette tip to draw the sample from the well. FIG. 25 illustrates a well 280 having a dimple-shaped construction and ribs that guide the pipette tip to the dimple. FIG. 26 illustrates a well 290 similar to the well 280 shown in FIG. 25 with posts which create many meniscuses exposing a larger surface area of the sample to the air to dry quickly. FIG. 27 illustrates a well 300 having a bottom surface that is sufficiently wide to dry the sample and sufficiently deep to receive a tip of a pipette. FIG. 28 illustrates another well 310 that is sufficiently deep to receive a tip of a pipette and can be tilted to spread sample over large side surface for efficient drying.

### Sponge and Plunger Collection Device

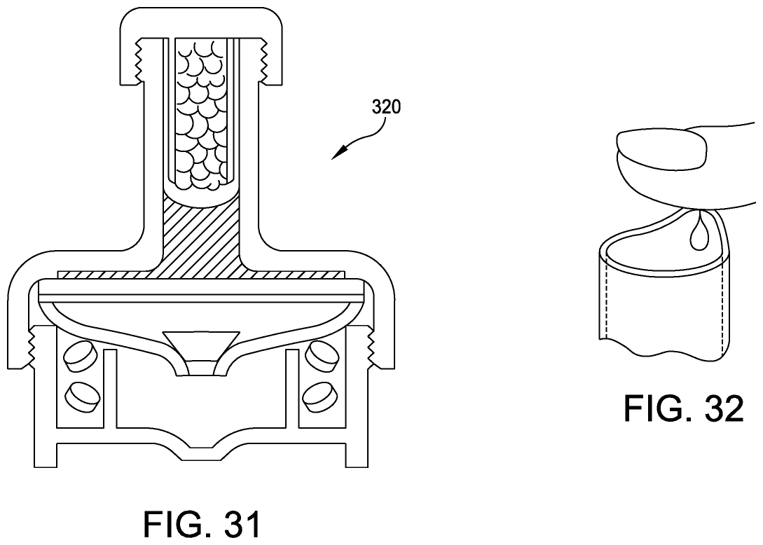

Referring to FIGS. 29-32, a sample collection device 320 includes a container and a removable cap. The container includes a membrane, a capillary disfunnel, a silk cake, desiccant capsules and a well for dry-down. The removable cap includes an absorbent material to absorb excess biological sample. During use, a user fills container or vial until a sample is above a line, then secures the removable cap. As discussed, the removable cap is filled with an absorbent material that extends to a precise distance, wicking away excess sample to achieve a precise sample volume. If gravity and capillary action from insufficient for plasma separation, the absorbent cap can be removed and a plunger cap can be applied to provide positive pressure to the sample forcing it through the membrane and into a funnel. In the funnel, sample mixes with a silk formulation cake, then enters well for dry-down. The user can ship the collection device after waiting one hour. Transparent sides provide the user the ability to inspect the sample for sufficient drying before shipping to prevent shipping with wet sample that may exit the well during shipping if device is turned upside-down.

### Sponge and Vacuum Collection Device

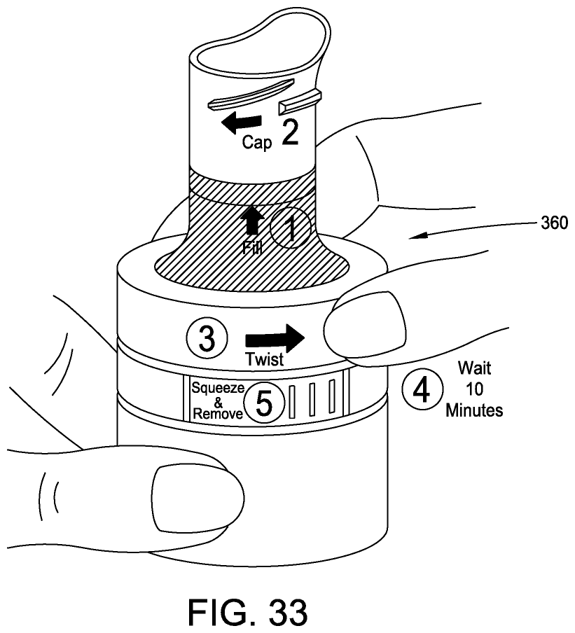

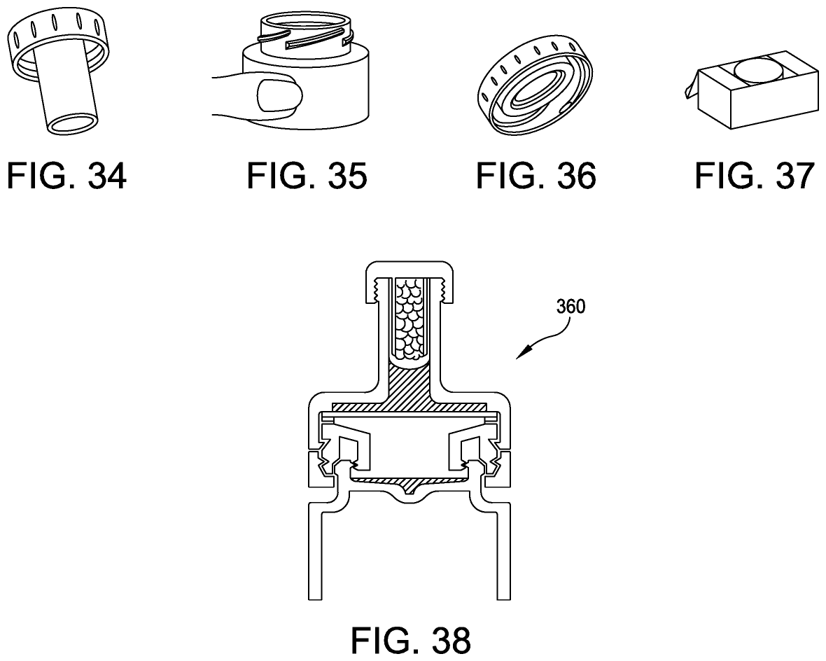

Referring to FIGS. 33-37, a sample collection device 360 includes a container and a removable cap. After lancing the finger, the user fills the vial until it is at or above a line. Next, the user secures the cap with absorbent material onto the container, and immediately twists the top part of the device with respect to the bottom until the top vial part hits a stop. After waiting ten minutes, the user squeezes the container to release a middle ring and unscrews top vial assembly to separate it from the well. Next, the user screws a desiccant cap onto the well and inverts the well several times, e.g., ten times, and then places the collection device in a box for shipping which helps maintain the well in an upright position during shipping to avoid tipping the sample well upside down before sample is dry. This maintains sample in bottom of well during drying to ensure full reconstitution in the lab; to ensure no part of the sample ends up on sides or cap where it will not be easily reconstituted. In a lab, well is used for reconstitution.

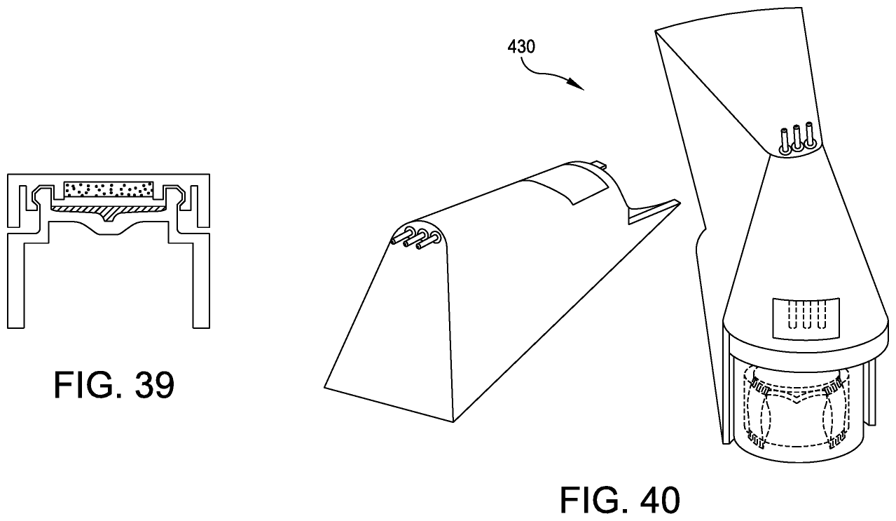

Referring to FIGS. 38 and 39, blood is deposited into the container, which is coated with anticoagulant, until the blood is above the line. Once filled the container is capped with the removable cap. The cap is filled with an absorbent material that extends to a precise distance, wicking away excess sample to achieve a precise sample volume. The user unscrews top vial part until detent to move a plunger linearly providing negative pressure in a well or chamber, drawing plasma/serum through a membrane. The plasma/serum enters a well with lyophilized silk formulation cake and anticoagulant. The user squeezes and removes release ring, removing thread stop and enabling vial assembly to be unscrewed from well. The user caps the well with desiccant-filled cap separated from sample by a vapor-permeable membrane. The user inverts well multiple times (e.g., ten times) to ensure sample is well-mixed with silk formulation and anticoagulant. In the lab, the cap is removed and well is used for reconstitution.

### Capillary Tilt Collection Device

Referring to FIG. 40, a sample collection device 430 includes packaging having three capillaries and a window to view ends of the capillaries. The packaging further includes a simple cup or other container to collect a sample. The device holds capillaries at a convenient distance from a table to enable precise blood collection volume by an inexperienced user. Blood is deposited into the capillaries until all three are completely full as shown through window. The capillary device is tilted over a sample processing device that may include all or some of plasma separation, mixing with anticoagulant, mixing with silk, and well for dry down, shipping, and reconstitution. The blood empties out of the capillaries and into the device through force of gravity. The capillary tilt device is removed and discarded. The device has only two flat sides to make it intuitive how to orient it; one side for blood collection, and another side for depositing blood in sample processing device.

In some embodiments, a sample collection device is configured to move directly from sample collection to plasma separation steps without reducing sample volume by transferring vessels, and reducing user steps for ease of use at home, and accommodating large fingerstick samples, e.g., greater than 100 ul.

In some embodiments, a sample collection device includes a vial with plasma separation membrane as bottom surface.

In some embodiments, a sample collection device includes a vial where the sample can pass through bottom by removing a bottom or revealing holes through user rotation or linear motion, thereby allowing sample to be in contact with a plasma separation membrane.

In some embodiments, a sample collection device is configured to provide precise sample metering and homogenous mixing with silk and anticoagulant before deposit onto a sample processing device.

In some embodiments, a sample collection device is configured to collect blood in a capillary or vial with lyophilized silk formulation and anticoagulant and dispensed onto a device with plasma separation and other sample processing features. This embodiment provides precise metering of sample, and homogenous mixing with silk and anticoagulant before deposit.

In some embodiments, a sample collection device is configured to enable an untrained home user to perform quality plasma separation with acceptable (low) levels of hemolysis and maximizing plasma yield from whole blood sample.

In some embodiments, a sample collection device is configured to apply positive pressure above the sample by enabling the user to rotate a threaded part with a plunger above the sample in a well or in a capillary, with a plasma separation membrane on bottom. Positive pressure is applied by pushing the sample through a membrane. Alternatively, the user pushes linearly on a plunger until it hits a detent or bends or flexes a non-permeable rubber or metal to create positive pressure above the sample.

In some embodiments, a sample collection device is configured to apply negative pressure below sample. In this embodiment, the device includes a connected sample well, a membrane, and a collection well. The user rotates a threaded part until hitting a thread stop or snap detent which pulls back a plunger or bends or flexes a non-permeable material, such as a rubber or metal snap dome, to create a negative pressure in the well, thereby pulling the sample through the membrane.

In some embodiments, a sample collection device is configured to keep a plasma sample in the same container between plasma separation, drying, shipping, and reconstitution in lab, thereby reducing losses in sample volume due to transfer to other containers, and decreasing use steps for untrained user at home.

In some embodiments, a sample collection device is configured so that the constituent parts of the device, including a container or well and a plasma separation assembly, are enclosed in a sealed container with desiccant for shipping. Desiccant is separated physically from the plasma separation assembly, but can interact through small air opening through which desiccant cannot pass. Or, in another embodiment, desiccant is separated from the plasma separation assembly by a vapor-permeable membrane which will not allow liquid to pass. The laboratory that processes the sample removes the plasma separation assembly to access and reconstitute sample upon arrival.

In some embodiments, a sample collection device is configured so that a plasma separation assembly that can be easily detached from a plasma sample well by an untrained home user by removing a thread stop from the device, or by unscrewing the plasma separation assembly from device. A cap with desiccant can be added to well. Desiccant is separated from the plasma separation assembly by a vapor-permeable membrane which will not allow liquid to pass.

In some embodiments, a sample collection device is configured to provide homogeneous mixing with silk formulation.

In some embodiments, a sample collection device is configured to include a silk formulation that is lyophilized in a capillary coated with anticoagulant. The sample is passed through the capillary to provide homogeneous mixing.

In some embodiments, a sample collection device is configured to include a silk formulation that is delivered in cake format in the sample collection device after plasma separation.

In some embodiments, a sample collection device is configured to use the same sample well for sample collection, drying, shipping, reconstitution, and analysis. The well can provide continuous sample tracking from remote sample collection setting to the lab by patient labeling (e.g., patient name, date, patient ID number, barcode, 2D barcode) to keep the sample linked to a patient at all steps of the process.

In some embodiments, a sample collection device is configured to use the same sample well for sample collection, drying, shipping, reconstitution, and analysis. The well enables smaller sample collection as it limits sample losses that occur when transferring samples between multiple containers.

In some embodiments, a sample collection device is configured to enable an untrained home user to dry the sample to the extent it is no longer mobile, and it is ready to ship.

In some embodiments, a sample collection device is configured to include posts positioned at a bottom of a well to create more meniscuses that lead to greater sample surface area exposed to air and promote quicker drying.

In some embodiments, a sample collection device is configured to dry a sample quickly, easily and in an error-free manner by a home user, but provides a well deep enough for receiving a tip of a pipette during reconstitution in lab.

In some embodiments, a sample collection device is configured to include various well constructions. In one embodiment, the well is an Eppendorf well, having a flat side of the well. In one embodiment, a user lays the sample in a tilted holder to spread sample on flat side to maximize surface area exposed to air to reduce drying time and increase profundity of drying. In the laboratory, the well remains tipped in same orientation during reconstitution with water, and then tipped upright to move sample to an Eppendorf area that is optimized for pipette access for mixing and analysis.

In some embodiments, a sample collection device is configured so that a well of the device has a flat bottom to spread the sample as much as possible, with a small dimple in middle to enable pipette access for mixing and analysis.

### Kit Packaging

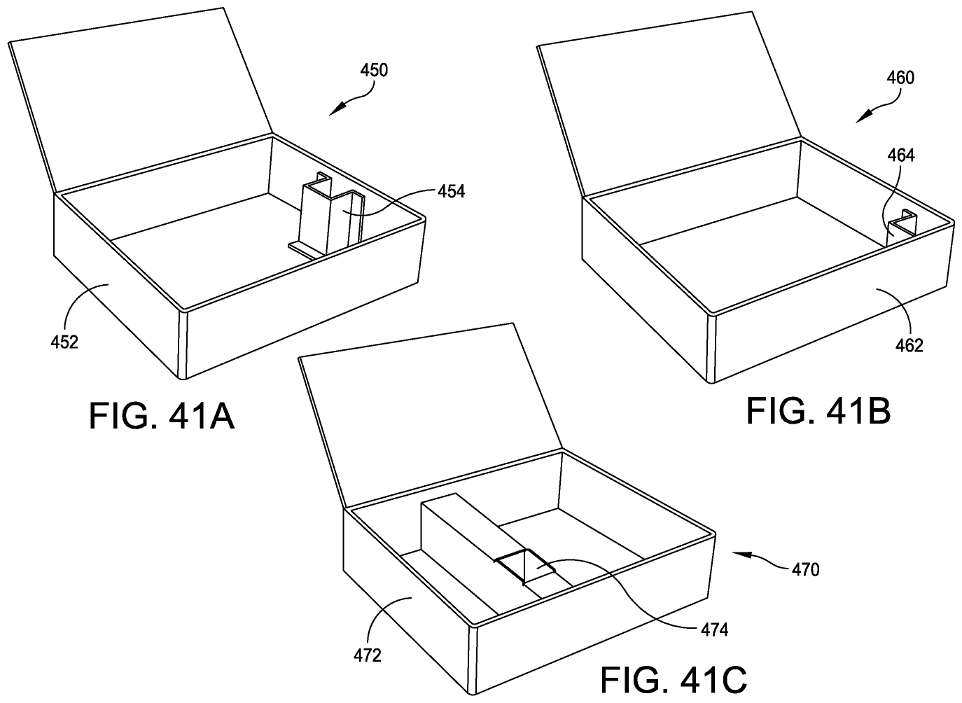

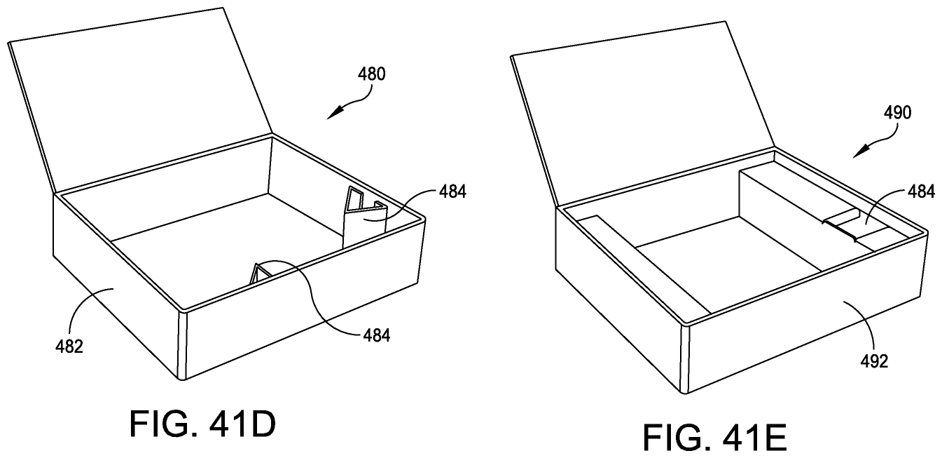

Referring to FIG. 41A, kit packaging that is configured to transport a sample collection device, such as sample collection device 150 described above, is generally indicated at 450. As shown, the kit packaging 450 includes a generally rectangular-shaped box 452 having a paperboard tube rack 454 incorporated into the box. In the shown embodiment, the tube rack 454 includes a square-shaped opening, and is positioned centrally within the box 452 along a side of the box. The tube rack 454 is configured to support the sample collection device in an upright position during plasma separation and dry down.

Referring to FIG. 41B, kit packaging is generally indicated at 460. As shown, the kit packaging 460 includes a generally rectangular-shaped box 462 having a paperboard tube rack 464 incorporated into the box. In this embodiment, the tube rack 464 includes a square-shaped opening, and is positioned in a corner of the box 462. The tube rack 464 is configured to support the sample collection device in an upright position during plasma separation and dry down.

Referring to FIG. 41C, kit packaging is generally indicated at 470. As shown, the kit packaging 470 includes a generally rectangular-shaped box 472 having a paperboard tube rack 474 incorporated into the box. In the shown embodiment, the tube rack 474 includes a square-shaped opening, and is positioned centrally within the box 452 and extends from one side of the box to an opposite side of the box. The tube rack 474 is configured to support the sample collection device in an upright position during plasma separation and dry down.

Referring to FIG. 41D, kit packaging is generally indicated at 480. As shown, the kit packaging 480 includes a generally rectangular-shaped box 482 having one or more paperboard tube racks, each indicated at 484, incorporated into the box. In the shown embodiment, each tube rack 484 includes a triangular-shaped opening, and is positioned along a side of the box 452. The paperboard tub rack 484 is configured to be sized to flex slightly when receiving a sample collection device to make a snug fit with the device. The tube rack 484 is configured to support the sample collection device in an upright position during plasma separation and dry down.

Referring to FIG. 41E, kit packaging is generally indicated at 490. As shown, the kit packaging 490 includes a generally rectangular-shaped box 492 having a paperboard tube rack 494 incorporated into the box. In the shown embodiment, the tube rack 494 includes a square-shaped opening, and is positioned along a side of the box 492 and extends from one side of the box to an opposite side of the box. The tube rack 494 is configured to support the sample collection device in an upright position during plasma separation and dry down. The tube rack 494 can include a top surface that forms a shelf to store instructions, for example.

### II. Biological Samples and Analytes Thereof

The devices, polymer compositions, and methods provided herein may be used to collect and/or to stabilize a biological sample, e.g., during shipping and/or storage as may occur prior to the biological sample being analyzed according to assays and instrumentation known in the art and described herein. Accordingly, the invention provides for improved human health and disease screening, diagnosis, monitoring, and optimization (e.g., health optimization). In particular, the devices, polymer compositions, and methods provided herein may be used to collect, stabilize, and enable the analysis of a biological sample, or analyte thereof, relevant to the evaluation, monitoring, identification, and/or treatment of a disease, a disorder, a condition, a syndrome, and/or a symptom thereof. In some embodiments, the disease, disorder, condition, syndrome, and/or symptom thereof, comprises a cardiovascular disease, a cancer, diabetes (e.g., type 1 diabetes and/or type 2 diabetes), a renal disease, a liver disease, a lysosomal storage disease, a pulmonary disease, a musculoskeletal disease, an endocrine disorder, an immunological disorder, an inflammatory disorder, a metabolic disorder, a chronic obstructive pulmonary disease, a sexually transmitted disease (STD), and/or an infectious disease (e.g., a bacterial infection, a viral infection, a fungal infection, and/or a parasite infection (e.g., a protozoan infection)). In some embodiments, the devices, polymer compositions, and methods provided herein may be used to collect, stabilize, and enable the analysis of a biological sample, or analyte thereof, for the purposes of a health and/or wellness screen (e.g., an evaluation of the metabolism, the dietary intake, and/or the hormone levels of a subject). In some embodiments, the devices, polymer compositions, and methods provided herein may be used to collect, stabilize, and enable the analysis of a biological sample, or analyte thereof, for the purposes of screening for a sexually transmitted disease (STD).

The biological samples described herein may be a composition that is obtained or derived from a subject that comprises a fraction, a component, and/or an analyte (e.g., a cellular, chemical, and/or other molecular entity as described herein) that is to be characterized and/or identified, for example, based on physical, biochemical, chemical, and/or physiological characteristics by laboratory assays and/or instrumentation known in the art. The biological sample may be from a subject that would be expected to or is known to contain the analyte that is to be characterized. In some embodiments, the subject is a mammal, including not limited to, domesticated animals (e.g., cows, sheep, cats, dogs, and horses), primates (e.g., humans and non-human primates such as monkeys), rabbits, and rodents (e.g., mice and rats). In certain embodiments, the subject is a human.

Exemplary biological samples, including, but are not limited to, a bodily fluid, e.g. whole blood, blood plasma, blood serum, urine, feces, saliva, amniotic fluid, and cerebrospinal fluid, breast milk, sputum, tears, ear wax, red blood cells, buffy coat, semen, serous fluid, cells and/or tissues, e.g. a buccal swab sample, a vaginal swab sample, a biopsy material, such as a tumor biopsy sample, and/or a cell culture samples, e.g., a eukaryotic cell culture sample and/or a bacterial cell culture sample.

Exemplary analytes include proteins, nucleic acids, pathogens (e.g., viruses), small molecules, electrolytes, elements, pathogens, lipids, carbohydrates, organic compounds, dissolved gases, plasticizers, environmental hazards, vitamins, polymers, and combinations thereof.

In some embodiments, the analyte may be a protein. Protein analytes may include, but are not limited to, a biomarker, an antibody, a growth factor, a cytokine, a globular protein, a structural protein, and/or an enzyme. In some embodiments the protein is an enzyme (e.g., a liver enzyme, e.g., an alanine aminotransferase, an alkaline phosphatase, and/or an aspartate aminotransferase).

In some embodiments, the analyte may be a biomarker, such as a e.g. a cancer biomarker as described herein. Exemplary cancer biomarkers include, but are not limited to, alpha fetoprotein (AFP), CA15-3, CA27-29, CA19-9, CA-125, Calcitonin, Calretinin, Carcinoembryonic antigen, CD34, CD99MI2, CD117, Chromogranin, Chromosomes 3, 7, 17, and 9p21, Cytokeratin (various types: TPA, TPS, Cyfra21-1), Desmin, Epithelial membrane antigen (EMA), Factor VIII, CD31 FL1, Glial fibrillary acidic protein (GFAP), Gross cystic disease fluid protein (GCDFP-15), HMB-45, Human chorionic gonadotropin (hCG), immunoglobulin, inhibin, keratin(s), lymphocyte marker(s), MART-1 (Melan-A), Myo D1, muscle-specific actin (MSA), neurofilament, neuron-specific enolase (NSE), placental alkaline phosphatase (PLAP), prostate-specific antigen (PSA), PTPR(CD45), S100 protein, smooth muscle actin (SMA), synaptophysin, thymidine kinase, thyroglobulin (Tg), thyroid transcription factor-1 (TTF-1), Tumor M2-PK, and vimentin.

In some embodiments, the analyte may be one typically included in a metabolic panel, e.g., a comprehensive metabolic panel (CMP). In some embodiments, the metabolic panel is a commercially available metabolic panel, such as a commercially available CMP. Exemplary analytes that may be included in a metabolic panel, e.g., a CMP, include, but are not limited to albumin (ALB), blood urea nitrogen (BUN), calcium (CA), carbon dioxide (bicarbonate; CO2), chloride (CL), creatinine (CREAT), glucose (GLUC), potassium (K), sodium (NA), total bilirubin (BIL), total protein (TP), alanine aminotransferase (ALT), alkaline phosphatase (ALP), aspartate aminotransferase (AST), cholesterol (CHOL), high-density lipoproteins (HDL), triglycerides (TRIG), globulin, phosphorus (P), eGFR, and/or c-reactive protein (CRP).

In some embodiments, the analyte may be one typically included in a cardiac risk assessment panel (e.g., c-reactive protein (CRP)).

In some embodiments, the analyte may be one typically included in an electrolyte panel (e.g., carbon dioxide (bicarbonate; CO2), chloride (CL), potassium (K), and/or sodium (NA)).

In some embodiments, the analyte may be one typically included in a hepatic function panel (e.g., alanine aminotransferase (ALT), albumin (ALB), alkaline phosphatase (ALP), aspartate aminotransferase (AST), bilirubin (BIL), and/or total protein (TP)).

In some embodiments, the analyte may be one typically included in an acute hepatitis panel (e.g., Hepatitis A Antibody, IgM; Hepatitis B Core Antibody, IgM; Hepatitis B Surface Antigen; and/or Hepatitis Virus Antibody).

In some embodiments, the analyte may be one typically included in a lipid panel (e.g., cholesterol (CHOL), high-density lipoproteins (HDL), low-density lipoproteins (LDL), very low-density lipoproteins (VLDL), and/or triglycerides (TRIG)). In some embodiments, a lipid panel may include a measurement of an LDL:HDL ratio. In some embodiments, a lipid panel may include a measurement of a total cholesterol:HDL ratio.

In some embodiments, the analyte may be one typically included in a renal function panel (e.g., albumin (ALB), blood urea nitrogen (BUN), creatinine (CREAT), BUN: CREAT ratio, calcium (CA), calcium (CA), carbon dioxide (bicarbonate; CO2), chloride (CL), glucose (GLUC), phosphorus (P), potassium (K), and/or sodium (NA)).

In some embodiments, the analyte may be one typically included in an obstetric panel (e.g., ABO Grouping; antigen and/or antibody for HIV-1 or HIV-2, CB with platelet count and differential, HBsAg screen, Rh factor, rubella antibodies, and/or syphilis serology).

In some embodiments, a cancer biomarker may be identified by standard methods, such as by the use of a tumor marker test and/or a cancer panel. Without wishing to be bound by theory, a tumor marker test and/or a cancer panel can be used to measure the presence, the level, and/or the activity of a protein or a gene in a biological sample (e.g., a tissue sample, a whole blood sample, a blood plasma sample, a blood serum sample, or a sample comprising a bodily fluid) that may indicate the development and/or progression of a cancer.

In some embodiments, the analyte may be a risk marker of developing cardiovascular disease, such as e.g. troponin I, troponin T, B-type natriuretic peptide (BNP), NT-proBNP, sICAM-1, Myeloperoxidase, CRP, fibrinogen, serum amyloid P, Von Willebrand Factor (vWF), α-2-macroglobulin, L-selectin, Apo AI, Apo AII, Apo B, Apo CII, Apo CIII, and/or Apo E.

In some embodiments, the analyte may be an antibody, such as an IgM, IgG, IgE, IgA, and/or IgD antibody. In some embodiments, the antibody may be to a pathogen (e.g., a virus, a parasite, and/or a bacteria), a chemical, and/or a toxin, e.g., an antibody relevant to screening for an infection and/or immunity.

In some embodiments, the antibody may be to a virus. Exemplary viruses include, but are not limited to, a polio virus, a rotavirus, a flavivirus, an influenza virus, a measles virus, a mumps virus, a rubella virus, a herpes virus, a cytomegalovirus, a mycoplasma, an adenovirus, a human immunodeficiency virus (HIV).

In some embodiments, the analyte is a vaccine-elicited neutralizing antibody.

In some embodiments, the analyte may be an enzyme, such as e.g. a lysosomal storage disorder enzyme. Exemplary lysosomal storage enzymes include, but are not limited to α-Galactosidase A (GLA), Acid β-glucosidase (GBA), β-Galactocerebrosidase (GALC), α-L-iduronidase (IDUA), Iduronate-2-sulfatase (IDS), N-acetyl-galactosamine-6-sulfatase (GALNS), N-acetylgalactosamine-4-sulfatase (Arylsulfatase B, ARSB), Acid sphingomyelinase (ASM), and Acid α-glucosidase (GAA).

In some embodiments, the analyte may be a nucleic acid. Exemplary nucleic acids analytes include, but are not limited to, genomic DNA (gDNA), viruses (e.g., a DNA virus and/or an RNA virus), mRNA (e.g., associated with a parasite, such as a malaria parasite), and/or portions or fragments thereof.

In some embodiments, the analyte is a nucleic acid (e.g., an mRNA) associated with a parasite, such as a malaria parasite.

Exemplary small molecule analytes include, but are not limited to, small molecules associated with environmental hazards (e.g., bisphenol A (BPA), perfluorooctanoic acid (PFOA), and/or perfluorooctanesulfonic acid (PFOS)), hormones (e.g., cortisol, progesterone, 11-dehydrocorticosterone, and 21-hydroxypregnenolone), organic compounds (e.g., trimethylamine N-oxide (TMAO)), and metabolites (e.g., α-ketoglutarate).

Exemplary elements include, but are not limited to, lead, arsenic, mercury, calcium, iron, magnesium, potassium, sodium, and zinc.

### III. Stabilizing Compositions and Formulations

The present invention discloses, at least in part, polymer-based biological sample collection devices that are configured to enable the formation of a substantially dried polymer composition, e.g., a silk fibroin composition (e.g., a silk film), comprising a biological sample, that has surprisingly increased stability over time and/or at elevated temperatures, e.g., as compared to a reference sample.

The stabilizing polymer compositions described herein can be formulate to stabilize, e.g., the structure, integrity, configuration, function, and/or activity of a biological sample, or at least one fraction, component, and/or analyte thereof for at least about 1 day (e.g., about 1 day, about 2 days, about 3 days, about 4 days, about 5 days, about 6 days, about 7 days), e.g., at a temperature between about 4° C. and about 45° C. (e.g., about 4° C., about 10° C., about 15° C., about 20° C., about 25° C., about 30° C., about 35° C., about 37° C., about 40° C., or about 45° C.). The polymer compositions described herein can be formulate to stabilize a biological sample, or at least one analyte thereof, for at least about 1 week (e.g., about 1 week, about 2 weeks, about 3 weeks, about 1 month, about 2 months, about 3 months, about 4 months, about 5 months, about six months, about 7 months, about 8 months, about 9 months, about 10 months, about 11 months, about 1 year, or longer), e.g., at a temperature between about 4° C. and about 45° C. (e.g., about 4° C., about 10° C., about 15° C., about 20° C., about 25° C., about 30° C., about 35° C., about 37° C., about 40° C., or about 45° C.). The polymer compositions described herein can stabilize the structure, integrity, configuration, function, and/or activity of a biological sample, or at least one fraction, component, and/or analyte thereof to enable long-term storage (e.g., for biobanking purposes, e.g., for at least about 1 year or longer, e.g., at a temperature between about 4° C. and about 45° C.).

Stabilization of a biological sample, or at least on analyte thereof can be evaluated by downstream analysis using standard laboratory assays and instrumentation. In some embodiments, the polymer-based biological sample collection devices are configured to be compatible with such standard laboratory assays and instrumentation. In some embodiments, the instrumentation is a clinical chemical analyzer. In some embodiments, the instrumentation is an LC-MS/MS instrument, an NMR instrument, and/or an rt-PCR instrument.

The stabilizing polymer compositions described herein can comprise a natural or a synthetic polymer. In some embodiments, the polymer comprises and/or is derived from a peptide or a polypeptide. In some embodiments, the polymer comprises and/or is derived from a silk fibroin protein, a gelatin protein, an albumin protein, an elastin protein, a collagen protein, a heparin protein, and/or a cellulose protein. In some embodiments, the polymer comprises and/or is derived from polyethylene glycol (PEG), polyethylene oxide (PEO), hyaluronic acid, carboxymethylcellulose (CMC), polyvinylpyrrolidone (PVP), and/or polyvinyl alcohol (PVA). However, any suitable natural polymer, synthetic polymer, or biodegradable polymer (e.g., medical grade polymer) may be used in a polymer composition described herein.

In some embodiments, about 0.01 mg to about 100 mg of a stabilizing polymer composition (e.g., silk fibroin composition) (e.g., about 0.05 mg to about 80 mg, about 0.75 mg to about 50 mg, about 0.75 mg to about 6 mg, about 1 mg to about 50 mg, about 1 mg to about 25 mg, about 1 mg to about 10 mg, about 2 to about 10 mg, about 3 to about 6 mg, or about 0.01 mg to about 10 mg) can be incorporated into a device described herein, e.g., as substantially dried (e.g., lyophilized, air-dried, or spray dried), or a liquid formulation.

In some embodiments, the stabilizing polymer composition (e.g., silk fibroin composition) is disposed at location or surface of a device described herein, e.g., in a sample collection and mixing portion, e.g., a capillary tube. In some embodiments, the substantially dried compositions described herein can comprise about 0.01 mg to about 10 mg (e.g., about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, or about 10 mg) of a polymer (e.g., silk fibroin).

In some embodiments, the polymer composition (e.g., a silk fibroin composition) can comprise about 0.01% w/v to about 50% w/v of a polymer (e.g., silk fibroin) (e.g., about 0.01% w/v, about 0.02% w/v, about 0.03% w/v, about 0.04% w/v, about 0.05% w/v, about 0.06% w/v, about 0.07% w/v, about 0.08% w/v, about 0.09% w/v, about 0.1% w/v, about 0.2% w/v, about 0.3% w/v, about 0.4% w/v, about 0.5% w/v, about 0.6% w/v, about 0.7% w/v, about 0.8% w/v, about 0.9% w/v, about 1% w/v, about 2% w/v, about 3% w/v, about 4% w/v, about 5% w/v, about 6% w/v, about 7% w/v, about 8% w/v, about 9% w/v, about 10% w/v, about 11% w/v, about 12% w/v, about 13% w/v, about 14% w/v, about 15% w/v, about 16% w/v, about 17% w/v, about 18% w/v, about 19% w/v, about 20% w/v, about 21% w/v, about 22% w/v, about 23% w/v, about 24% w/v, about 25% w/v, about 26% w/v, about 27% w/v, about 28% w/v, about 29% w/v, about 30% w/v, about 31% w/v, about 32% w/v, about 33% w/v, about 34% w/v, about 35% w/v, about 36% w/v, about 37% w/v, about 38% w/v, about 39% w/v, about 40% w/v, about 41% w/v, about 42% w/v, about 43% w/v, about 44% w/v, about 45% w/v, about 46% w/v, about 47% w/v, about 48% w/v, about 49% w/v, or about 50% w/v), e.g., before drying.

A polymer (e.g., silk fibroin) as described herein, can be present in the device (e.g., in the stabilizing polymer composition) in an amount of about 0.1% w/v to about 20% w/v (weight of polymer to volume of biological sample) (e.g., about 0.5% w/v to about 15% w/v, about 0.75% w/v to about 10% w/v, about 1% w/v to about 5% w/v, about 1% w/v to about 3% w/v, about 1% w/v to about 2% w/v, or about 5% w/v to about 10% w/v polymer).

In some embodiments, the polymer composition can comprise about 25 μL to about 300 μL (e.g., about 25 μL, about 30 μL, about 35 μL, about 40 μL, about 45 μL, about 50 μL, about 55 μL, about 60 μL, about 65 μL, about 70 μL, about 75 μL, about 80 μL, about 85 μL, about 90 μL, about 95 μL, about 100 μL, about 125 μL, about 150 μL, about 175 μL, about 200 μL, about 225 μL, about 250 μL, about 275 μL, about 300 μL) of a polymer described herein, e.g., a silk fibroin composition, e.g., before drying.

When the polymer composition is a silk fibroin composition, the silk fibroin composition can be a low molecular weight silk fibroin composition comprising a population of silk fibroin fragments having a range of molecular weights, characterized in that: no more than 15% of the total number of silk fibroin fragments in the population has a molecular weight exceeding 200 kDa, and at least 50% of the total number of the silk fibroin fragments in the population has a molecular weight within a specified range, wherein the specified range is between about 3.5 kDa and about 120 kDa, or between about 5 kDa and about 125 kDa.

Stated another way, the silk fibroin solution used in the fabrication of a device described herein can comprise a population of silk fibroin fragments having a range of molecular weights, characterized in that: no more than 15% of the total moles of silk fibroin fragments in the population has a molecular weight exceeding 200 kDa, and at least 50% of the total moles of the silk fibroin fragments in the population has a molecular weight within a specified range, wherein the specified range is between about 3.5 kDa and about 120 kDa, or between about 5 kDa and about 125 kDa. (see, e.g., WO2014/145002, incorporated herein by reference herein).

In some instances, the substantially dried polymer composition can comprises a silk fibroin composition comprising a population of silk fibroin having an average molecular weight of about 1 kDa to about 250 kDa (e.g., about 1 kDa to about 20 kDa, about 20 kDa to about 40 kDa, about 40 kDa to about 60 kDa, about 60 kDa to about 80 kDa, about 80 kDa to about 100 kDa, about 100 kDa to about 120 kDa, about 120 kDa to about 140 kDa, about 140 kDa to about 160 kDa, about 160 kDa to about 180 kDa, about 180 kDa to about 200 kDa), e.g., before drying.

In some embodiments, the silk fibroin compositions can be prepared, e.g., according to established methods. In some embodiments, pieces of cocoons from the silkworm Bombyx mori were first boiled in 0.02 M Na₂CO₃ to remove sericin protein which is present in unprocessed, natural silk, prior to analysis by SEC.

In some embodiments, the silk fibroin composition can be a composition or mixture produced by degumming silk cocoons (e.g., silk cocoons from the silkworm Bombyx mori) at an atmospheric boiling temperature (e.g., in an aqueous sodium carbonate solution) for about 480 minutes or less, e.g., less than 480 minutes, less than 400 minutes, less than 300 minutes, less than 200 minutes, less than 180 minutes, less than 120 minutes, less than 100 minutes, less than 60 minutes, less than 50 minutes, less than 40 minutes, less than 30 minutes, less than 20 minutes, less than 10 minutes or shorter. In some embodiments, the silk fibroin composition may be a 10 minute boil (10 MB) silk fibroin composition, a 30 minute boil (30 MB) silk fibroin composition, a 60 minute boil (60 MB) silk fibroin composition, a 120 minute boil (120 MB) silk fibroin composition, a 180 minute boil (180 MB) silk fibroin composition, a 240 minute boil (240 MB) silk fibroin composition, a 300 minute boil (300 MB) silk fibroin composition, a 360 minute boil (360 MB) silk fibroin composition, a 420 minute boil (420 MB) silk fibroin composition, and/or a 480 minute boil (480 MB) silk fibroin composition produced accordingly to a method known in the art and/or described herein. silk fibroin composition or mixture.

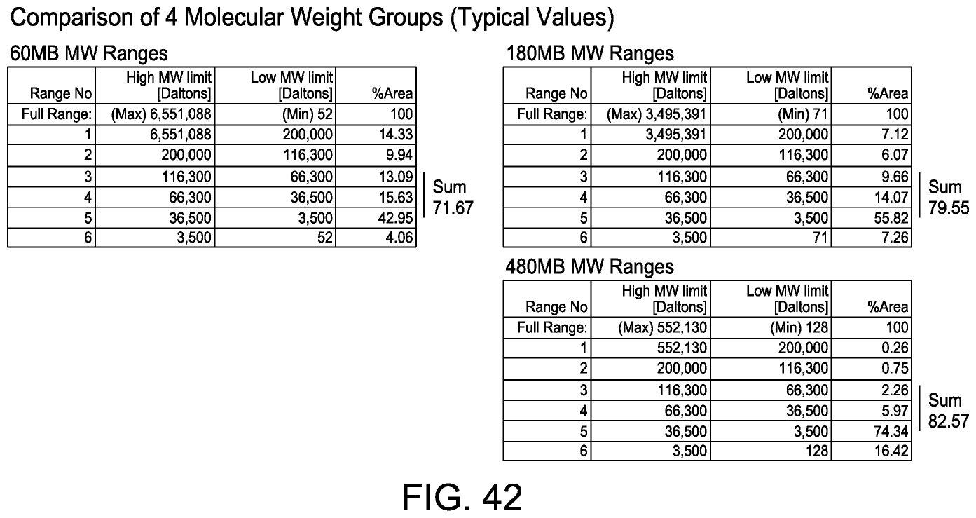

In some instances, the 30 MB silk fibroin composition can comprise a population of silk fibroin having an average molecular weight of about 250 kDa (e.g., 246.9±16.9 kDa). In some instances, the 60 MB silk fibroin composition can be defined according to FIG. 42 and/or comprises a population of silk fibroin having an average molecular weight of about 150 kDa (e.g., 152.8±11.6 kDa). In some instances, the 120 MB silk fibroin composition can comprise a population of silk fibroin having an average molecular weight of about 100 kDa (e.g., 99.7±0.02 kDa). In some instances, the 180 MB silk fibroin composition can be defined according to FIG. 42 and/or comprises a population of silk fibroin having an average molecular weight of about 70 kDa (e.g., 70.5±0.38 kDa). In some instances, the 480 MB silk fibroin composition can be defined according to FIG. 42 and/or comprises a population of silk fibroin having an average molecular weight of about 36 kDa (e.g., 35.9±0.03 kDa). Exemplary silk fibroin (e.g., regenerated silk fibroin) compositions may have different molecular weight profiles, e.g., as determined by size exclusion chromatography (SEC) methods (see, e.g., FIG. 42).

The substantially dried polymer composition can comprise, e.g., a population of silk fibroin having an average dispersity index (e.g., polydispersity index (PDI)) of about 5 to about 20 (e.g., about 5, about 6, about 7, about 8 about 9, about 10, about 11, about 12, about 13, about 14, about 15, about 16, about 17, about 18, about 19, about 20), e.g., before drying.

In some embodiments, the silk fibroin composition comprises silk fibroin in an amount less than 10% (w/v), less than 9% (w/v), less than 8% (w/v), less than 7% (w/v), less than 6% (w/v), less than 5% (w/v), less than 4% (w/v), less than 3.5% (w/v), less than 3% (w/v), less than 2.5% (w/v), less than 2% (w/v), less than 1.5% (w/v), less than 1% (w/v), less than 0.5% (w/v), less than 0.1% (w/v), but greater than 0.001% (w/v), e.g., before drying (e.g., air drying). In some embodiments, silk fibroin is present in an amount between about 0.1% (w/v) to about 3% (w/v), about 1.5% (w/v) to about 2.8% (w/v), or about 2% (w/v) and about 2.5% (w/v), e.g., before drying.

In some embodiments, a biological sample described herein can be formulated in a 1% w/v to about 10% w/v (e.g., about 1%, about 2%, about 3%, about 4%, about 5%, about 6%, about 7%, about 8%, about 9%, or about 10% w/v) 60 MB silk fibroin solution (see, e.g., FIG. 42), e.g., before drying. In some embodiments, a biological sample described herein can be formulated in a 1% w/v to about 10% w/v (e.g., about 1%, about 2%, about 3%, about 4%, about 5%, about 6%, about 7%, about 8%, about 9%, or about 10% w/v) 120 MB silk fibroin solution (see, e.g., FIG. 42), e.g., before drying. In some embodiments, a biological sample described herein can be formulated in a 1% w/v to about 10% w/v (e.g., about 1%, about 2%, about 3%, about 4%, about 5%, about 6%, about 7%, about 8%, about 9%, or about 10% w/v) 180 MB silk fibroin solution (see, e.g., FIG. 42), e.g., before drying. In some embodiments, a biological sample described herein can be formulated in a 1% w/v to about 10% w/v (e.g., about 1%, about 2%, about 3%, about 4%, about 5%, about 6%, about 7%, about 8%, about 9%, or about 10% w/v) 480 MB silk fibroin solution (see, e.g., FIG. 42), e.g., before drying.

In some embodiments, the polymer-based biological sample collection devices comprise and/or are configured to enable the formation of a substantially dry polymer, e.g., silk fibroin composition, wherein the polymer composition, e.g., silk fibroin composition, can be in any form, including, but not limited to, a film, a fiber, a particle, a gel, a hydrogel, a solution, a foam, a sponge, a mat, a mesh, a fabric, a powder, a coating layer, a porous scaffold, a lyophilized form, or any combinations thereof.

In some embodiments, the polymer-based biological sample collection devices are configured to enable the formation of a substantially dry polymer composition comprising a silk fibroin protein, a gelatin protein, an albumin protein, an elastin protein, a collagen protein, a heparin protein, and/or a cellulose protein. In some embodiments, the polymer-based biological sample collection devices are configured to enable the formation of a substantially dry polymer composition comprising a polyethylene glycol (PEG), polyethylene oxide (PEO), hyaluronic acid, carboxymethylcellulose (CMC), polyvinylpyrrolidone (PVP), and/or polyvinyl alcohol (PVA) comprising a biological sample.

In other embodiments, the polymer-based biological sample collection devices are configured to enable the formation of a silk fibroin composition (e.g., a silk matrix) comprising a biological sample that are in liquid form.

In some embodiments, the polymer fibroin composition (e.g., a silk matrix) comprising a biological sample further comprises an excipient as described herein. The silk fibroin composition (e.g., a silk matrix) comprising a biological sample can be produced in the polymer-based biological sample collection devices described herein, e.g., by forming a solution with a polymer (e.g., a lyophilized silk), a biological sample, an excipient, and/or a divalent cation, and substantially drying the resulting solution by air-drying and/or by desiccant facilitated air-drying, as described herein.

### Excipients

The polymer compositions, e.g., silk fibroin compositions, used in the fabrication of the devices described herein may further include an excipient. In some embodiments, inclusion of an excipient may be for the purposes of improving the stability of a collected biological sample, or an analyte thereof. In some embodiments, an excipient described herein may be included to expedite drying (e.g., air drying and/or desiccant facilitated air drying). In some embodiments, an excipient described herein may be included to improve re-solubility of a substantially dried composition as described herein, e.g., prior to analysis.

Exemplary excipients include, but are not limited to, a sugar and/or a sugar alcohol (e.g., sucrose, trehalose, maltose, sorbitol, mannitol, glycerol, or a combination thereof), a protein (e.g., a silk fibroin protein, a gelatin protein, an albumin protein, an elastin protein, a collagen protein, a heparin protein, and/or a cellulose protein), a polymer (e.g., polyethylene glycol (PEG), polyethylene oxide (PEO), hyaluronic acid, carboxymethylcellulose (CMC), polyvinylpyrrolidone (PVP), polyvinyl alcohol (PVA)), a divalent cation (e.g., Ca²⁺, Mg²⁺, Mn²⁺, and Cu²⁺), a salt (e.g., monosodium glutamate), a buffer (e.g., a Tris buffer, a Tris EDTA buffer, a citrate buffer, a phosphate buffer, a MOPS buffer, and/or a HEPES buffer), a hyaluronate, an amino acid (e.g., L-glutamic acid and/or lysine), and solvents (e.g., DMSO, methanol, and/or ethanol).

An excipient as described herein can be present in the device (e.g., in the polymer composition) in an amount less than 70% w/v (e.g., less than 70% w/v, less than 60% w/v, less than 50% w/v, less than 40% w/v, less than 30% w/v, less than 20% w/v, less than 10% w/v, less than 9% w/v, less than 8% w/v, less than 7% w/v, less than 6% w/v, less than 5% w/v, or less than 1% w/v), optionally before drying. In some embodiments, the excipient can be present in an amount between about 1% w/v to about 10% w/v (e.g., about 1% w/v, about 2% w/v, about 3% w/v, about 4% w/v, about 5% w/v, about 6% w/v, about 7% w/v, about 8% w/v, about 9% w/v, or about 10% w/v), optionally before drying.

An excipient as described herein can be present in the device (e.g., in the polymer composition) in an amount of about 0.01% w/v to about 50% w/v (e.g., about 0.01% w/v, about 0.02% w/v, about 0.03% w/v, about 0.04% w/v, about 0.05% w/v, about 0.06% w/v, about 0.07% w/v, about 0.08% w/v, about 0.09% w/v, about 0.1% w/v, about 0.2% w/v, about 0.3% w/v, about 0.4% w/v, about 0.5% w/v, about 0.6% w/v, about 0.7% w/v, about 0.8% w/v, about 0.9% w/v, about 1% w/v, about 2% w/v, about 3% w/v, about 4% w/v, about 5% w/v, about 6% w/v, about 7% w/v, about 8% w/v, about 9% w/v, about 10% w/v, about 11% w/v, about 12% w/v, about 13% w/v, about 14% w/v, about 15% w/v, about 16% w/v, about 17% w/v, about 18% w/v, about 19% w/v, about 20% w/v, about 21% w/v, about 22% w/v, about 23% w/v, about 24% w/v, about 25% w/v, about 26% w/v, about 27% w/v, about 28% w/v, about 29% w/v, about 30% w/v, about 31% w/v, about 32% w/v, about 33% w/v, about 34% w/v, about 35% w/v, about 36% w/v, about 37% w/v, about 38% w/v, about 39% w/v, about 40% w/v, about 41% w/v, about 42% w/v, about 43% w/v, about 44% w/v, about 45% w/v, about 46% w/v, about 47% w/v, about 48% w/v, about 49% w/v, or about 50% w/v), e.g., before drying.

An excipient, as described herein, can be present in the device (e.g., in the stabilizing polymer composition, e.g., silk fibroin composition) in an amount of about 0.1% w/v to about 20% w/v excipient (weight of excipient to volume of biological sample) (e.g., about 0.5% w/v to about 15% w/v, about 0.75% w/v to about 10% w/v, about 1% w/v to about 5% w/v, about 1% w/v to about 3% w/v, about 1% w/v to about 2% w/v, or about 5% w/v to about 10% w/v excipient).

In some embodiments, the polymer composition comprises silk fibroin as an excipient and the silk fibroin is present in an amount between about 0.1% w/v to about 20% w/v silk fibroin (weight of excipient to volume of biological sample) (e.g., about 0.5% w/v to about 15% w/v, about 0.75% w/v to about 10% w/v, about 1% w/v to about 5% w/v, about 1% w/v to about 3% w/v, about 1% w/v to about 2% w/v, or about 5% w/v to about 10% w/v gelatin), e.g., before drying.

In some embodiments, the polymer composition comprises gelatin as an excipient and the gelatin is present in an amount between about 0.1% w/v to about 20% w/v gelatin (weight of excipient to volume of biological sample) (e.g., about 0.5% w/v to about 15% w/v, about 0.75% w/v to about 10% w/v, about 1% w/v to about 5% w/v, about 1% w/v to about 3% w/v, about 1% w/v to about 2% w/v, or about 5% w/v to about 10% w/v gelatin), e.g., before drying.

In some embodiments, the polymer composition comprises albumin as an excipient and the albumin is present in an amount between about 0.1% w/v to about 20% w/v albumin (weight of excipient to volume of biological sample) (e.g., about 0.5% w/v to about 15% w/v, about 0.75% w/v to about 10% w/v, about 1% w/v to about 5% w/v, about 1% w/v to about 3% w/v, about 1% w/v to about 2% w/v, or about 5% w/v to about 10% w/v albumin), e.g., before drying.

In some embodiments, the polymer composition comprises elastin as an excipient and the elastin is present in an amount between about 0.1% w/v to about 20% w/v albumin (weight of excipient to volume of biological sample) (e.g., about 0.5% w/v to about 15% w/v, about 0.75% w/v to about 10% w/v, about 1% w/v to about 5% w/v, about 1% w/v to about 3% w/v, about 1% w/v to about 2% w/v, or about 5% w/v to about 10% w/v albumin), e.g., before drying.

In some embodiments, the polymer composition comprises collagen as an excipient and the collagen is present in an amount about 0.1% w/v to about 20% w/v collagen (weight of excipient to volume of biological sample) (e.g., about 0.5% w/v to about 15% w/v, about 0.75% w/v to about 10% w/v, about 1% w/v to about 5% w/v, about 1% w/v to about 3% w/v, about 1% w/v to about 2% w/v, or about 5% w/v to about 10% w/v collagen), e.g., before drying.

In some embodiments, the polymer composition comprises heparin as an excipient and the heparin is present in an amount about 0.1% w/v to about 20% w/v heparin (weight of excipient to volume of biological sample) (e.g., about 0.5% w/v to about 15% w/v, about 0.75% w/v to about 10% w/v, about 1% w/v to about 5% w/v, about 1% w/v to about 3% w/v, about 1% w/v to about 2% w/v, or about 5% w/v to about 10% w/v heparin), e.g., before drying.

In some embodiments, the polymer composition comprises hyaluronate as an excipient and the hyaluronate is present in an amount between about 0.1% w/v to about 20% w/v hyaluronate (weight of excipient to volume of biological sample) (e.g., about 0.5% w/v to about 15% w/v, about 0.75% w/v to about 10% w/v, about 1% w/v to about 5% w/v, about 1% w/v to about 3% w/v, about 1% w/v to about 2% w/v, or about 5% w/v to about 10% w/v hyaluronate), e.g., before drying.

In some embodiments, the polymer composition comprises a polymer (e.g., polyethylene glycol (PEG), polyethylene oxide (PEO), hyaluronic acid, carboxymethylcellulose (CMC), polyvinylpyrrolidone (PVP), and/or polyvinyl alcohol (PVA)) as an excipient and the polymer is present in an amount between about 0.1% w/v to about 20% w/v polymer (weight of excipient to volume of biological sample) (e.g., about 0.5% w/v to about 15% w/v, about 0.75% w/v to about 10% w/v, about 1% w/v to about 5% w/v, about 1% w/v to about 3% w/v, about 1% w/v to about 2% w/v, or about 5% w/v to about 10% w/v polymer).

In some embodiments, the polymer composition comprises a polymer (e.g., a protein polymer, e.g., silk fibroin) as an excipient and the polymer is present in an amount between about 0.1% (w/v) to about 10% (w/v) (weight of excipient to volume of biological sample) (e.g., about 0.2% (w/v) to about 8% (w/v), or about 0.4% (w/v) to about 6% (w/v), about 0.5% (w/v) to about 3% (w/v), about 0.6% (w/v) to about 2.8% (w/v), about 0.8% (w/v) and about 2.5%, or about 0.1%, or about 2.4% (w/v)).

In some embodiments, the polymer composition comprises a sugar and/or a sugar alcohol as an excipient. In some embodiments, the sugar and/or a sugar alcohol excipient is sucrose, and the sucrose is present in silk fibroin composition in an amount less than 70% (w/v), less than 60% (w/v), less than 50% (w/v), less than 40% (w/v), less than 30% (w/v), less than 20% (w/v), less than 10% (w/v), less than 9% (w/v), less than 8% (w/v), less than 7% (w/v), less than 6% (w/v), less than 5% (w/v), less than 1% (w/v) or less, e.g., before drying.

In some embodiments, the sugar or the sugar alcohol is sucrose present in an amount between about 0.1% w/v to about 20% w/v (weight of excipient to volume of biological sample) (e.g., about 0.5% w/v to about 15% w/v, about 0.75% w/v to about 10% w/v, about 1% w/v to about 5% w/v, about 1% w/v to about 3% w/v, about 1% w/v to about 2% w/v, or about 5% w/v to about 10% w/v sugar or sugar alcohol).

In some embodiments, the sugar or the sugar alcohol is sucrose present in an amount between about 1% (w/v) to about 10% (w/v) (e.g., about 2% (w/v) to about 8% (w/v), about 2.2% (w/v) to about 6% (w/v), about 2.4% (w/v) to about 5.5% (w/v), about 2.5 to about 5%, or about 2.4% (w/v), about 2.5%, or about 5% (w/v)), e.g., before drying.

In some embodiments, the sugar or the sugar alcohol is trehalose present in an amount between about 1% (w/v) to about 10% (w/v), about 2% (w/v) to about 8% (w/v), about 2.2% (w/v) to about 6% (w/v), about 2.4% (w/v) to about 5.5% (w/v), about 2.5 to about 5%, or about 2.4% (w/v), about 2.5%, or about 5% (w/v), e.g., before drying.

In some embodiments, the sugar or the sugar alcohol is sorbitol present in an amount between about 1% (w/v) to about 10% (w/v), about 2% (w/v) to about 8% (w/v), about 2.2% (w/v) to about 6% (w/v), about 2.4% (w/v) to about 5.5% (w/v), about 2.5 to about 5%, or about 2.4% (w/v), about 2.5%, or about 5% (w/v), e.g., before drying.

In some embodiments, the sugar or the sugar alcohol is glycerol present in an amount between about 1% (w/v) to about 10% (w/v), about 2% (w/v) to about 8% (w/v), about 2.2% (w/v) to about 6% (w/v), about 2.4% (w/v) to about 5.5% (w/v), about 2.5 to about 5%, or about 2.4% (w/v), about 2.5%, or about 5% (w/v), e.g., before drying.

In some embodiments, the polymer composition further comprises a divalent cation. In some embodiments, the divalent cation is selected from the group consisting of Ca²⁺, Mg²⁺, Mn²⁺, and Cu²⁺. In some embodiments, the divalent cation is present in the silk fibroin composition before drying in an amount between 0.1 mM and 100 mM. In some embodiments, the divalent cation is present in the silk fibroin composition before drying in an amount between 10⁻¹⁰ to 2×10⁻³ moles.

In some embodiments, the substantially dried polymer composition further comprising a buffer, e.g., before drying. In some embodiments, the buffer has buffering capacity between pH 3 and pH 8, between pH 4 and pH 7.5, or between pH 5 and pH 7. In some embodiments, the buffer is at a concentration of about 1 mM to about 300 mM (e.g., about 1 mM to about 10 mM, about 10 mM to about 20 mM, about 20 mM to about 30 mM, about 30 mM to about 40 mM, about 40 mM to about 50 mM, about 50 mM to about 60 mM about 60 mM to about 70 mM, about 70 mM to about 80 mM, about 80 mM to about 90 mM, about 90 mM to about 100 mM, about 100 mM to about 125 mM, about 125 mM to about 150 mM, about 150 mM to about 175 mM, about 175 mM to about 200 mM, about 200 mM to about 225 mM, about 225 mM to about 250 mM, about 250 mM to about 275 mM, or about 275 mM to about 300 mM). In some embodiments, the buffer is present in the preparation before drying in an amount between 0.1 mM and 100 mM. In some embodiments, the buffer is selected from the group consisting of a MOPS buffer, a HEPES buffer, and a CP buffer. In some embodiments, the buffer is present in an amount between 10⁻¹⁰ to 2×10⁻³ moles.

### IV. Methods of Use

The invention also provides methods for using the polymer-based biological sample collection devices described herein. The invention features a method of making a substantially dried composition, which may be optionally further processed into a reaction mixture and/or an analysis sample for downstream analysis, e.g., by the use of standard laboratory assays and/or instrumentation as described herein.

The method can include the steps of: (i) metering a pre-determined amount of a biological sample (e.g., a whole blood, a blood serum, and/or blood plasma sample) into a device described herein, wherein the device comprises a polymer composition as described herein (e.g., lyophilized silk fibroin composition); (ii) mixing (e.g., to homogeneity) the biological sample of (i) with the lyophilized silk fibroin composition of (i) to form a mixture; (iii) drying (e.g., air drying) the biological sample and/or the mixture, optionally, wherein the drying (e.g., air-drying) of the biological sample and/or the mixture occurs within about 48 hours (e.g., within about 1-5 minutes, e.g., within about 1-10 minutes, e.g., within about 1-20 minutes, e.g., within about 1-30 minutes, e.g., within about 1-40 minutes, e.g., within about 1-50 minutes, e.g., within about 1-60 minutes, e.g., within about 1, 2, 3, 4, 5, 6, 7, 8, 9, 10, 20, 30, 40, 50, or 60 minutes, e.g., within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours), thereby making a substantially dried composition.

In some embodiments, the substantially dried composition produced can retain the structure, integrity, configuration, function, and/or activity of a biological sample, or at least one fraction, component, and/or analyte thereof, for at least about 1 day (e.g., about 1 day, about 2 days, about 3 days, about 4 days, about 5 days, about 6 days, about 7 days), e.g., at a temperature between about 4° C. and about 45° C. (e.g., about 4° C., about 10° C., about 15° C., about 20° C., about 25° C., about 30° C., about 35° C., about 37° C., about 40° C., or about 45° C.). In some embodiments, the substantially dried composition produced can retain the structure, integrity, configuration, function, and/or activity of a biological sample, or at least one fraction, component, and/or analyte thereof, for at least about 1 week (e.g., about 1 week, about 2 weeks, about 3 weeks, about 1 month, about 2 months, about 3 months, about 4 months, about 5 months, about six months, about 7 months, about 8 months, about 9 months, about 10 months, about 11 months, about 1 year, or longer), e.g., at a temperature between about 4° C. and about 45° C. (e.g., about 4° C., about 10° C., about 15° C., about 20° C., about 25° C., about 30° C., about 35° C., about 37° C., about 40° C., or about 45° C.).

The invention also features methods of identifying and/or measuring the level of at least one fraction, component, and/or analyte of a biological sample. Using a device as described herein, the a fraction, component, and/or analyte of a biological sample can be identified and/or measured according to the following steps: (i) metering a pre-determined amount of a biological sample (e.g., a whole blood, a blood serum, and/or blood plasma sample) into a device described herein, wherein the device comprises a silk fibroin composition as described herein (e.g., lyophilized silk fibroin composition); (ii) mixing (e.g., to homogeneity) the biological sample of (i) with the lyophilized silk fibroin composition of (i) to form a mixture; (iii) drying (e.g., air drying) the biological sample and/or the mixture, optionally, wherein the drying (e.g., air-drying) of the biological sample and/or the mixture occurs within about 48 hours (e.g., within about 1-5 minutes, e.g., within about 1-10 minutes, e.g., within about 1-20 minutes, e.g., within about 1-30 minutes, e.g., within about 1-40 minutes, e.g., within about 1-50 minutes, e.g., within about 1-60 minutes, e.g., within about 1, 2, 3, 4, 5, 6, 7, 8, 9, 10, 20, 30, 40, 50, or 60 minutes, e.g., within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours), thereby making a substantially dried composition; (iv) reconstituting the substantially dried composition of (iii) in water, a buffer (e.g., PBS, optionally further comprising a surfactant and/or a sugar), an organic solvent, or a combination thereof to form an analyte sample (e.g., a reaction mixture and/or an analysis sample); (v) optionally, performing an extraction step on the analyte sample to form an extraction sample; (vi) optionally, performing a precipitation step on the analyte sample to form a precipitation sample; (vii) optionally, performing an enrichment step on the analyte sample to form an enrichment sample; (viii) optionally, performing a combination of steps (v)-(vii) to form a combination sample; and (ix) analyzing the analyte sample of (iv), the extraction sample of (v), the precipitation sample of (vi), the enrichment sample of (vii), and/or the combination sample of (viii) using laboratory assays and/or instrumentation known in the art or as described herein, thereby identifying and/or measuring the level of at least one fraction, component, and/or analyte of a biological sample.

In some embodiments, the enrichment step comprises a column enrichment, e.g., by an RNeasy spin column, when the analytes are nucleic acids such as mRNA and/or gDNA. The silk-based biological sample collection devices described herein can be configured to be used with and/or integrated with standard laboratory analysis assays and/or instrumentation. Without wishing to be bound by theory the devices of the invention can be configured to be compatible with and/or can be integrated with standard laboratory assays and/or instrumentation.

Various types of analyses can be performed on the analyte(s) of a biological sample, e.g., depending on the nature of the analyte of interest. Non-limiting examples of analyses can include, but are not limited to, mass spectroscopy, point of care platform tests, genotyping, nucleic acid sequencing, expression analysis (e.g., protein level, or transcript level), binding affinity, enzymatic activity, transfection efficiency, cell counting, cell identification, cell viability, immunogenicity, infectivity, metabolite profiling, bacterial screening (e.g., from fecal, urine, and cultures samples), and any combinations thereof. In some embodiments, at least one component of the biological sample can be subjected to at least one genotyping or nucleic acid sequencing analysis, expression analysis (e.g., protein level and/or transcript level), metabolite profiling, or any combinations thereof. Various methods to perform these analyses can include, but are not limited to, polymerase chain reaction (PCR), real-time quantitative PCR, microarray, western blot, immunohistochemical analysis, enzyme linked immunosorbent assay (ELISA), mass spectrometry, nucleic acid sequencing, flow cytometry, gas chromatography, high performance liquid chromatography, nuclear magnetic resonance (NMR) spectroscopy, or any combinations thereof. Techniques for nucleic acid sequencing are known in the art and can be used to assay the component to determine nucleic acid or gene expression measurements, for example, but not limited to, DNA sequencing, RNA sequencing, de novo sequencing, next-generation sequencing such as massively parallel signature sequencing (MPSS), polony sequencing, pyrosequencing, Illumina (Solexa) sequencing, SOLiD sequencing, ion semiconductor sequencing, DNA nanoball sequencing, Heliscope single molecule sequencing, single molecule real time (SMRT) sequencing), nanopore DNA sequencing, sequencing by hybridization, sequencing with mass spectrometry, microfluidic Sanger sequencing, microscopy-based sequencing techniques, RNA polymerase (RNAP) sequencing, or any combinations thereof.

In some embodiments, the subject is a human patient in need of a diagnosis of a disease and/or disorder. The disease and/or disorder may be selected from a cardiovascular disease, a cancer, diabetes (e.g., type 1 diabetes and/or type 2 diabetes), a renal disease, a liver disease, a lysosomal storage disease, a pulmonary disease, a musculoskeletal disease, an endocrine disorder, an immunological disorder, an inflammatory disorder, a metabolic disorder, a chronic obstructive pulmonary disease, a sexually transmitted disease (STD), and/or an infectious disease (e.g., a bacterial infection, a viral infection, a fungal infection, and/or a parasite infection (e.g., a protozoan infection)).

### V. Exemplary Kits

In certain embodiments, the invention relates to a package or kit comprising a device described herein. In some embodiments, the kits can further comprise a disinfectant (e.g., an alcohol swab). In some embodiments, the kits can further comprise a capillary tube (e.g., to be used for metering of the biological sample, e.g., outside the device), an metering device (e.g., a syringe, a cuvette, and/or a pipette), a lancet or other form of a venipuncture device, a bandage for wound closure, a bag to contain sample and/or to ship sample, an additional amount of a desiccant described herein, a gauze to wipe initial blood drop, a biohazard sealable foil pouch/bag (e.g., for storage and/or shipping), and/or instructions for use. In some embodiments, such packages, and kits described herein can be used for biological sample collection purposes.

It is to be appreciated that embodiments of the devices and methods discussed herein are not limited in application to the details of construction and the arrangement of components set forth in the following description or illustrated in the accompanying drawings. The devices and methods are capable of implementation in other embodiments and of being practiced or of being carried out in various ways. Examples of specific implementations are provided herein for illustrative purposes only and are not intended to be limiting. In particular, acts, elements and features discussed in connection with any one or more embodiments are not intended to be excluded from a similar role in any other embodiments.

Also, the phraseology and terminology used herein is for the purpose of description and should not be regarded as limiting. Any references to embodiments or elements or acts of the systems and methods herein referred to in the singular may also embrace embodiments including a plurality of these elements, and any references in plural to any embodiment or element or act herein may also embrace embodiments including only a single element. References in the singular or plural form are not intended to limit the presently disclosed systems or methods, their components, acts, or elements. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms. Any references to front and back, left and right, top and bottom, upper and lower, and vertical and horizontal are intended for convenience of description, not to limit the present systems and methods or their components to any one positional or spatial orientation.

Having thus described several aspects of at least one embodiment, it is to be appreciated various alterations, modifications, and improvements will readily occur to those skilled in the art. Such alterations, modifications, and improvements are intended to be part of this disclosure and are intended to be within the scope of the disclosure. Accordingly, the foregoing description and drawings are by way of example only, and the scope of the disclosure should be determined from proper construction of the appended claims, and their equivalents.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1. Lyophilization of Silk in Capillary Tubes

Methods:

Low molecular weight silk formulations of 1%, 2% and 4% w/v were pipetted into 1.75 mm diameter plastic capillary tubes coated with lithium heparin. Varying volumes of solution were pipetted into the tube to yield a final silk concentration of 1% w/v when mixed with 50 uL, 100 μL, or 150 μL of whole blood. Silk solutions in capillary tubes were lyophilized using a VirTis 25L Genesis SQ SuperXL-70 Freeze-Dryer (SP Scientific). Tubes were placed directly on a shelf of lyophilizer and frozen at −45 for 180 minutes. Primary drying occurred at −20 and 50 mT for 84 hours, and secondary drying occurred at 4 and 50 mT for 4 hours. Tubes were visually inspected to evaluate any gaps in drying. In addition, tubes were weighed before and after lyophilization to determine presence of any moisture in tubes. Once lyophilized, lithium heparinized whole blood (Research Blood Components) was pipetted into the capillary tubes and visually inspected to determine any issues with solubility.

### Results:

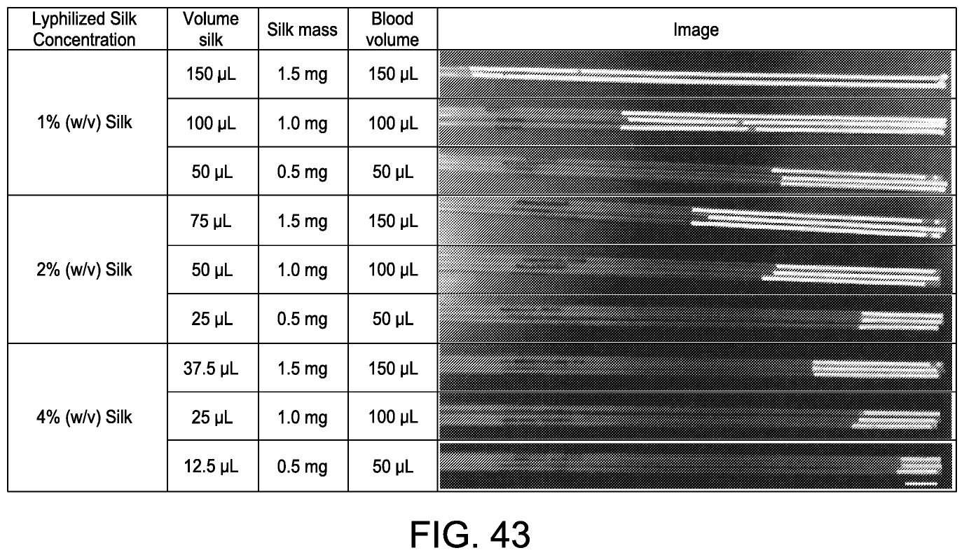

Various amounts of lyophilized silk were mixed with metered volumes of blood to evaluate the formation of a homogenously mixed 1%, 2%, and 4% (w/v) silk fibroin-blood solutions in a capillary tube having an internal diameter (ID) of 1.75 mm. To achieve the 1%, 2%, or 4% (w/v) silk fibroin-blood solution, the amount of blood was held constant to yield the indicated percentage after mixing with 50 μL, 100 μL, or 150 μL of blood (FIG. 43). FIG. 43 shows that a 1% (w/v) silk fibroin-blood solution can be formed, e.g., by fully dissolving the lyophilized silk, when 50 μL silk and 50 μL blood is mixed in a capillary tube having an internal diameter (ID) of 1.75 mm. Fully dissolved samples were observed in all volumes and formulations when whole blood was used.

### Example 2. Desiccant Drying Experiments

### Methods:

2.5 gram and 10 grams of Molecular Sieve (3 Å) were added to 20 mL scintillation vials. Plasma laden silk films were created by reconstituting silk with plasma at a 1:1 volume ratio (final silk concentration=1% w/v). Films (100 μL volume) were then cast onto 10 mm, 12 mm, and 14 mm polydimethylsiloxane discs and placed on top of the desiccant and sealed in the vial. The disks were weighed every hour for up to 8 hours to evaluate the amount of water loss in the film. Vials without any desiccant were used as a control.

### Results:

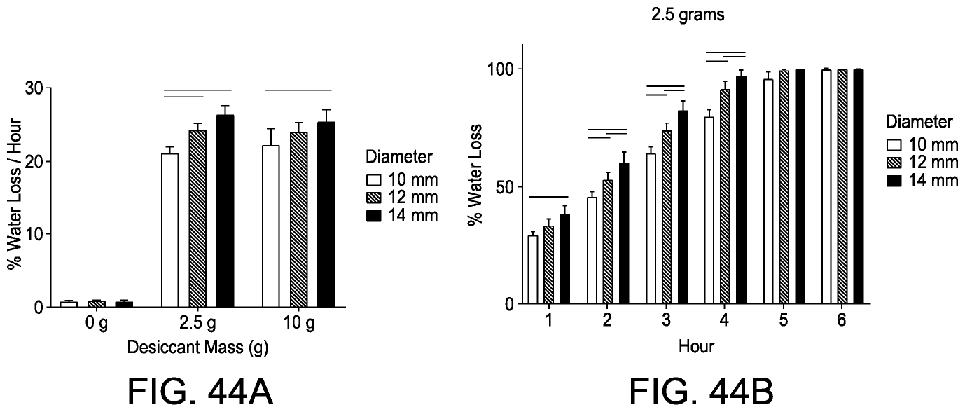

Various amounts of desiccant (e.g., 0 g, 2.5 g, and 10 g) were evaluated for their ability to dry a 100 μL volume of 1% w/v silk cast on a polydimethylsiloxane (PDMS) disk of diameter 10 mm, 12 mm, or 14 mm positioned within a sealed, 20 mL scintillation vial. Linear regression was used to determine the percent of water loss from the sample. It was determined that larger casting surface areas resulted in increased drying rates (FIG. 44A), however increased amount of desiccant from 2.5 g to 10 g did not result in different drying rates. The presence of desiccant, however, was required for the sample to dry completely (FIG. 44B).

### Example 3. Analyte Interference and Recovery Testing (Li-Hep)

### Methods:

Freshly drawn deidentified plasma containing lithium heparin anticoagulant was shipped from Research Blood Components (Brighton, Mass.). Upon receipt of samples, silk films were fabricated by mixing plasma with lyophilized silk solutions or silk/sucrose solutions at a 1:1 volume ratio. Plasma samples were cast onto 12 mm polydimethylsiloxane molds, and dried in a biosafety cabinet for 4 hours. Once dried, films were transferred to tubes and stored at 4° C. overnight. For analysis, samples were delivered to Boston Clinical Laboratories (Waltham, Mass.), where samples were reconstituted with deionized water at 1:1 volume ratio (200 μL water:200 μL plasma laden silk pre-dried). Once reconstituted, samples were run on a Beckman Coulter AU680 to determine total cholesterol (CHOL), high-density lipoprotein (HDL), triglyceride (TRIG), alkaline phosphatase (ALP), aspartate transaminase (AST), and alanine transaminase (ALT) concentrations. Samples were compared to liquid plasma samples stored overnight at 4° C. For interference testing, plasma was used to reconstitute lyophilized silk and silk/sucrose solutions at a 1:1 volume ratio. These samples were immediately processed on a Beckman Coulter AU680 on the same analytes listed above.

### Results:

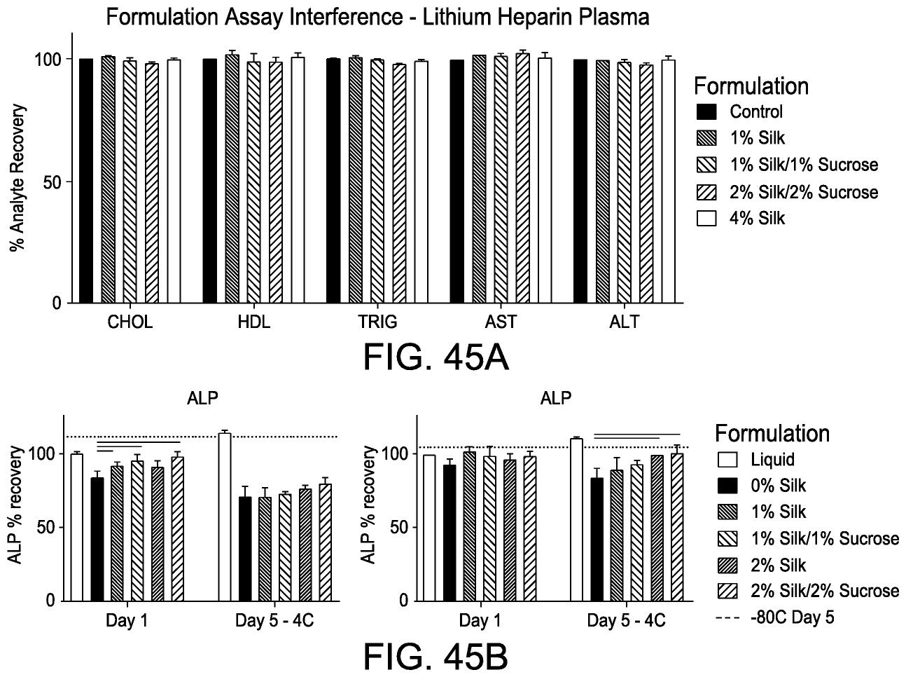

Compatibility of the formulation of the present devices were evaluated with a clinical chemistry analyzer (Beckman Coulter AU680) for relevant cardiovascular and liver disease biomarkers. These markers are routine tests completed in clinical laboratories, and include of total cholesterol (CHOL), high-density lipoprotein (HDL), triglyceride (TRIG), alkaline phosphatase (ALP), aspartate transaminase (AST), and alanine transaminase (ALT) levels. These tests are typically completed on liquid serum or plasma. In the present example, the analyzer was used to compare readings from formulated liquid plasma to assess assay interference, and formulated plasma films to further assess assay interference and recovery from a dried sample. It was determined that there was no interference of silk in the assay, however, an increase of sucrose concentration led to decreasing analyte recovery (FIG. 45A). This is due to a dilution effect from the higher solids. For dried films, however, recovery was analyte dependent with some analytes observing greater recovery in formulated films than others (FIG. 45B).

### Example 4. Evaluation of Alpha-Ketoglutarate (AKG) Stability

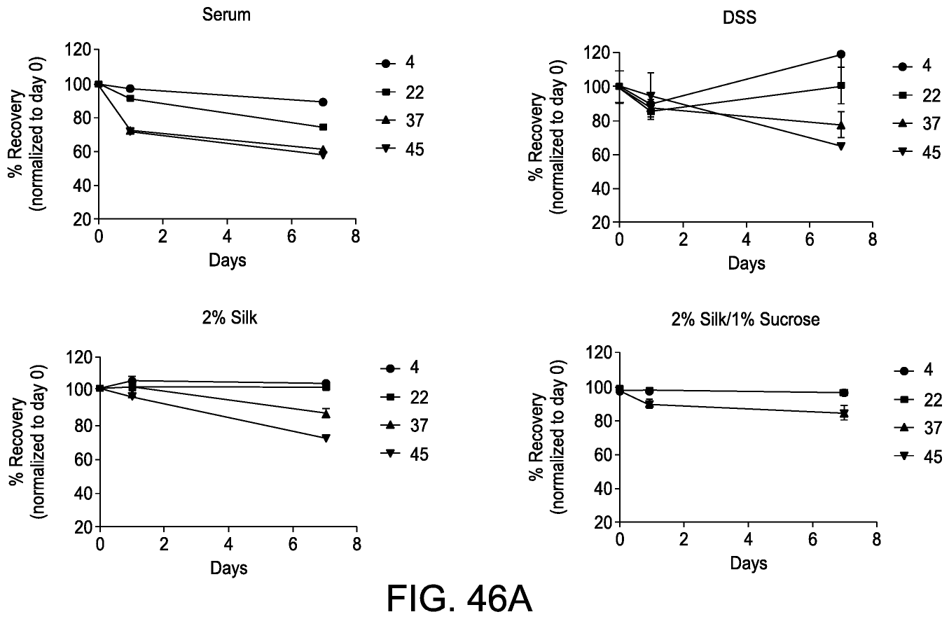

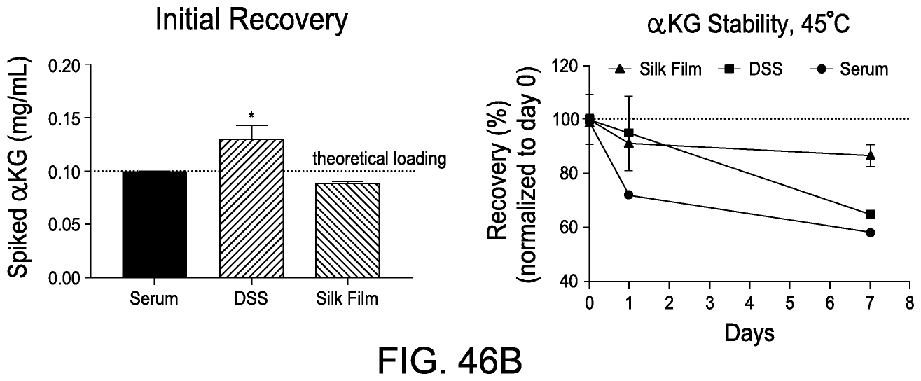

For assessing the stabilizing effect of silk fibroin compositions, a storage study was initiated at to assess the stability of alpha-ketoglutarate (AKG) stability in serum, dried serum spots (DSS), 2% silk film (also referred to as film A), 2% silk film comprising 1% sucrose (also referred to as film B) at 4° C., 22° C., 37° C., and 45° C. over 7 days (FIG. 46A). It was determined that DSS overestimates the concentration of aKG, possibly due to the approximation of the total serum amount in punch. Silk film recoveries were similar between Film A and Film B (FIG. 46B). It was determined that Film B outperformed DSS stability profile by 20% (FIG. 46B).

### Example 5. Analyte Interference and Recovery Testing (EDTA)

### Methods:

Freshly drawn deidentified plasma containing ethylenediaminetetraacetic acid (EDTA) anticoagulant was shipped from Research Blood Components (Brighton, Mass.). Upon receipt of samples, silk films were fabricated by mixing plasma with lyophilized silk solutions or silk/sucrose solutions at a 1:1 volume ratio. Plasma samples were cast onto 16 mm polydimethylsiloxane molds, and dried in a biosafety cabinet for 4 hours. Once dried, films were transferred to tubes and stored at 4° C. or 37° C. for five days. For analysis, samples were delivered to Boston Clinical Laboratories (Waltham, Mass.), where they were reconstituted with deionized water at 1:1 volume ratio (200 μL water:200 μL plasma laden silk pre-dried). Once reconstituted, samples were run on a Beckman Coulter AU680 to determine total cholesterol (CHOL), high-density lipoprotein (HDL), triglyceride (TRIG), alanine transaminase (ALT), gamma-glutamyl transferase (GGT), and C-reactive protein (CRP) concentrations. Samples were compared to liquid plasma samples stored for five days at −80° C., 4° C., and 37° C. For interference testing, plasma was used to reconstitute lyophilized silk and silk/sucrose solutions at a 1:1 volume ratio. These samples were immediately processed on a Beckman Coulter AU680 on the same analytes listed above.

### Results:

Compatibility of the formulation of the present devices were evaluated with a clinical chemistry analyzer (Beckman Coulter AU680) for relevant cardiovascular and liver disease biomarkers. These markers are routine tests completed in clinical laboratories, and include of total cholesterol (CHOL), high-density lipoprotein (HDL), triglyceride (TRIG), alanine transaminase (ALT), gamma-glutamyl transferase (GGT), and C-reactive protein (CRP) levels. These tests are typically completed on liquid serum or plasma. In the present example, the analyzer was used to compare readings from formulated liquid plasma to assess assay interference, and formulated plasma films to further assess assay interference and recovery from a dried sample.

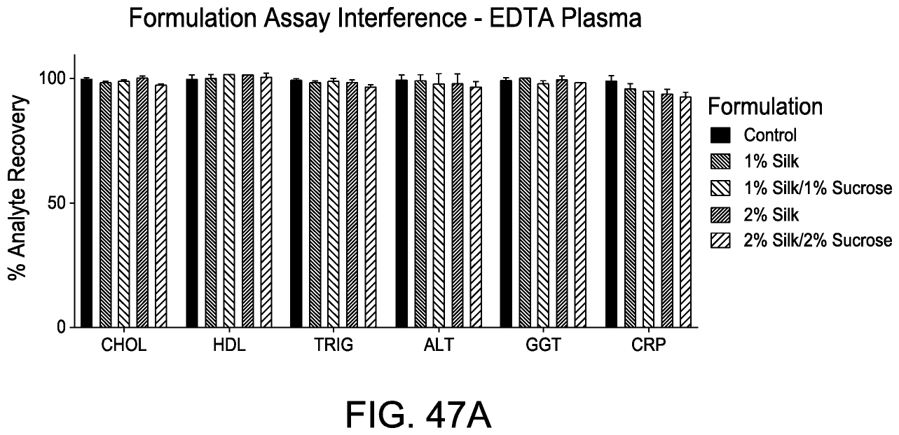

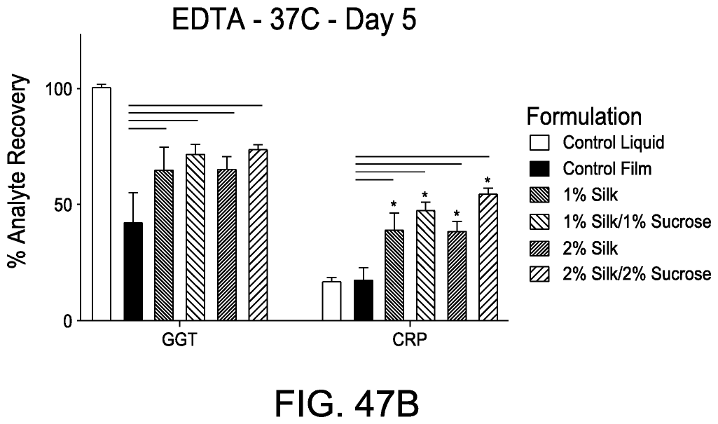

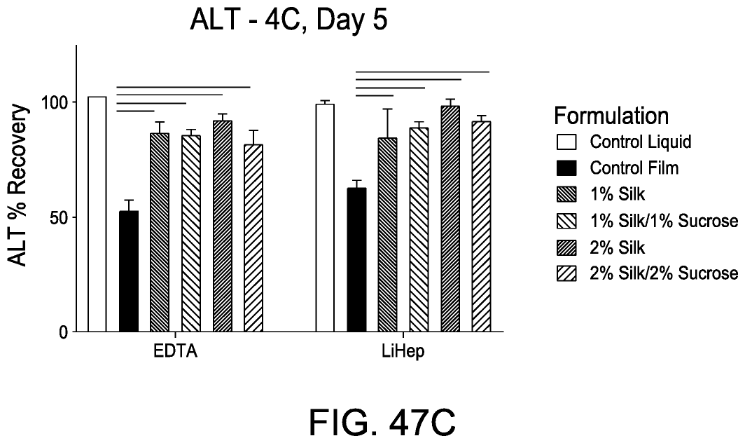

It was determined that there was no interference of silk in the assay, however, an increase of sucrose concentration led to decreasing analyte recovery (FIG. 47A). This is due to a dilution effect from the higher solids. For dried films, similar to the lithium heparin results, recovery was analyte dependent. When stored at 37° C. for 5 days, GGT and CRP exhibit different stability profiles. In both cases, however, formulated films were able to provide greater recovery than non-formulated films. Further, CRP recovery was significantly greater in formulated plasma films compared to the stored liquid control (FIG. 47B). Finally, stabilization with formulation was independent of anticoagulant (EDTA or lithium heparin), as observed with ALT recovery (FIG. 47C).

### Example 6. Formulated Blood Applied to Lateral Flow Separation Membranes

### Methods:

Different formulation compositions containing silk, sucrose, and HEPES were pipetted into 2.35 mm diameter plastic capillary tubes coated with lithium heparin. Varying volumes of solution were pipetted into the tube to yield a final silk concentration to assess drying rates. Formulation loaded capillary tubes were lyophilized using a VirTis 25L Genesis SQ SuperXL-70 Freeze-Dryer (SP Scientific). Tubes were placed directly on a shelf of the lyophilizer and frozen at −45° C. for 180 minutes. Primary drying occurred at −30 and 50 mT for 3.5 days, and secondary drying occurred at 4 and 50 mT for 1 hour. Tubes were visually inspected to evaluate any gaps in drying. In addition, tubes were weighed before and after lyophilization to determine presence of any moisture in tubes. Once lyophilized, lithium heparinized whole blood or heparinized plasma (Research Blood Components) was pipetted into the capillary tubes at a 1:1 volume ratio (50 uL blood:50 uL pre-lyophilized formulation). Formulation dissolution was assessed with the capillary tube being loaded in both the horizontal and vertical orientation.

### Results:

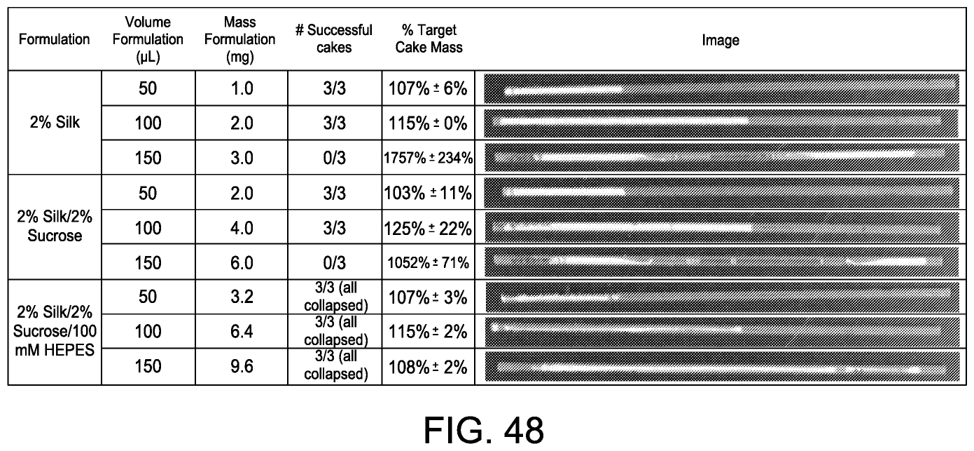

Various formulations were lyophilized and subsequently mixed with whole blood or plasma to assess solubility. As shown in FIG. 48, capillary tubes containing less than 100 uL formulation were able to successfully lyophilize under the used run conditions. Larger volumes, however, did not successfully completely lyophilize, as there was a gap of liquid formulation remaining in the middle of the tube. A longer primary dry duration would improve the success. When HEPES was incorporated into the formulation, the formulations were able to completely lyophilize regardless of loading volume, however the cakes were of poor quality and collapsed within the tube. Blood and plasma were also introduced into the tubes to assess solubility of the cakes. In all instances, both blood and plasma were able to completely dissolve the cake with the cake with tubes held in the vertical or horizontal orientation. Further, addition of sucrose increased the dissolution rate compared to the neat silk sample.

### Example 7. Formulated Blood Applied to Lateral Flow Separation Membranes

### Methods:

Freshly drawn deidentified whole blood containing lithium heparin anticoagulant was shipped from Research Blood Components (Brighton, Mass.). Upon receipt of sample, whole blood was used to reconstitute lyophilized silk/sucrose/HEPES solutions at a 1:1 volume ratio. Formulated or neat blood was directly pipetted onto the end of the an AdvanceDX100 (ADX100) or LF1 (GE Healthcare) paper membrane. Samples were allowed to separate and dry for four hours.

### Results:

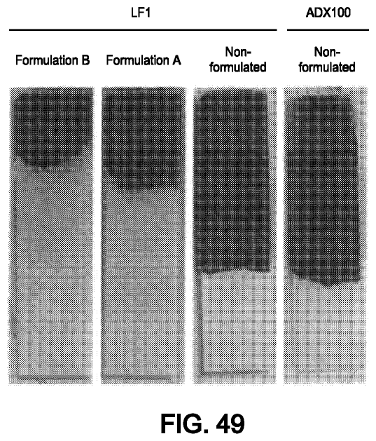

Whole blood samples were mixed with formulation and pipetted onto lateral flow plasma separation membranes to assess differences in separation efficiency compared to non-formulated blood. In this present example, two different formulations were tested with different silk/sucrose/HEPES concentrations in the range of 1-4% silk w/v. As shown in FIG. 49, when spotted onto two different types of membranes, non-formulated blood behaved similarly, with the distance the red blood cells traveled being similar between the ADX100 and LF1 cards. However, when formulation was incorporated into blood, the red blood cells remained more confined to the space where the blood was deposited, and the distance they traveled was significantly less than that of their non-formulated counterparts. In all instances, plasma fully travelled through to the end of the membrane. This data suggests that formulated blood would lead to increased allowable area for plasma to be punched, thereby allowing more testing from a single sample.

### Example 8. Buffered pH Drying

### Methods:

Freshly drawn deidentified whole blood containing lithium heparin anticoagulant was shipped from Research Blood Components (Brighton, Mass.). Upon receipt of sample, whole blood was centrifuged at 2000 g for 10 minutes to separate plasma. One part MOPS or HEPES buffer and 9 parts plasma were mixed to yield a final concentration of 0 mM, 25 mM, 50 mM, or 100 mM buffer in plasma. Plasma was cast onto 16 mm polydimethylsiloxane molds and allowed to dry. Once dry, films were transferred to tubes and reconstituted with deionized water at a 1:1 volume ratio (100 uL water:100 uL plasma pre-dried). During film drying between 0 min and 60 min, plasma was removed from the substrate and the pH of the same was measured. In addition, the pH of the reconstituted sample was measured at 180 min.

### Results:

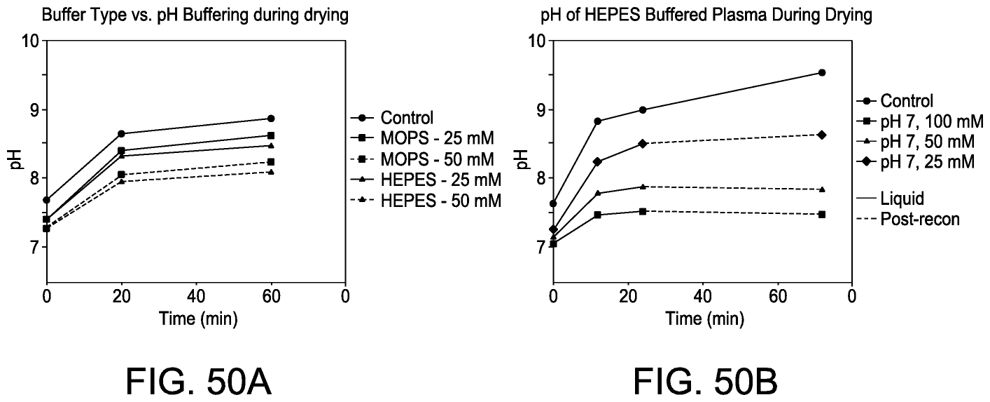

Plasma samples were mixed with MOPS or HEPES buffer to yield final concentrations between 0-100 mM buffer. The pH of the plasma samples was evaluated as the liquid samples underwent the drying process to create air-dried films. As shown in FIG. 50A HEPES buffer improved pH buffering capabilities slightly over MOPS buffer. As a result of this, higher concentrations of HEPES buffer, and pH of the films post-reconstitution was assessed. As shown in FIG. 50B, non-buffered plasma increases approximately 2 pH units as the plasma dries. Samples stored at high pH may experience stability issues and may also interfere with specific clinical assays. Incorporation of HEPES, however, decreases the delta pH in a dose dependent manner. Specifically, by incorporating 100 mM HEPES buffer into the formulation, the plasma film is allowed to dry and be stored in a physiological pH range.

### Example 9. Process Loss from Drying Mitigated Through pH Buffering

### Methods:

Freshly drawn deidentified whole blood containing lithium heparin anticoagulant was shipped from Research Blood Components (Brighton, Mass.). Upon receipt of sample, whole blood was centrifuged at 2000 g for 10 minutes to separate plasma. One part HEPES buffer and 9 parts plasma were mixed to yield a final concentration of 0 mM, 25 mM, 50 mM, or 100 mM HEPES in plasma. Plasma was cast onto 16 mm polydimethylsiloxane molds, and dried in a biosafety cabinet for 4 hours. Once dried, samples were transferred to tubes and reconstituted with deionized water at 1:1 volume ratio (100 uL water:100 uL plasma pre-dried). Samples were then analyzed for alkaline phosphatase (ALP) activity using a plate reader assay (Biovision, Cat. #: K412). ALP activity from films was compared to neat liquid plasma.

### Results:

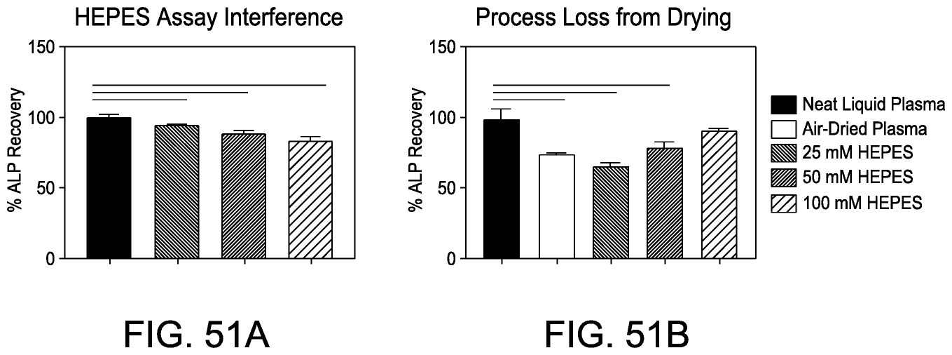

Plasma samples were mixed with HEPES buffer to yield final concentrations between 0-100 mM HEPES, before being cast onto disks and dried. Samples were immediately reconstituted, and assayed for alkaline phosphatase using a plate reader assay (Biovision, Cat. #: K412). As shown in FIG. 51B, it was determined that increasing buffer concentration in the plasma resulted in a reduction of process loss from drying. The presence of HEPES, however, may be interfering with the assay (FIG. 51A), as observed with a reduction in ALP recovery in the 25 mM HEPES film compared to non-formulated film.

### Example 10. Correlation Between Total Solids Loading and ALP Recovery

### Methods:

Freshly drawn deidentified whole blood containing lithium heparin anticoagulant was shipped from Research Blood Components (Brighton, Mass.). Upon receipt of samples, blood samples were centrifuged at 2000 g for 10 minutes to isolate plasma. Liquid plasma was isolated, mixed with different ratios of gelatin, sucrose, and HEPES using a design of experiments methodology to determine if solid type had a synergistic effect on analyte stability. Gelatin and sucrose concentrations in plasma ranged from 0-6%, and HEPES concentration remained at 100 mM, yielding final total solids in the plasma ranging between 2.4% and 14.4%. Formulated samples were cast onto 16 mm polydimethylsiloxane disks, dried, and stored at 37° C. overnight. For analysis, film samples were removed from stability and reconstituted with deionized water. Once in solution, plasma samples were assayed for alkaline phosphatase (ALP) using a plate reader assay (Biovision, Cat. #: K412). ALP recoveries were normalized to baseline/Day 0 liquid plasma.

### Results:

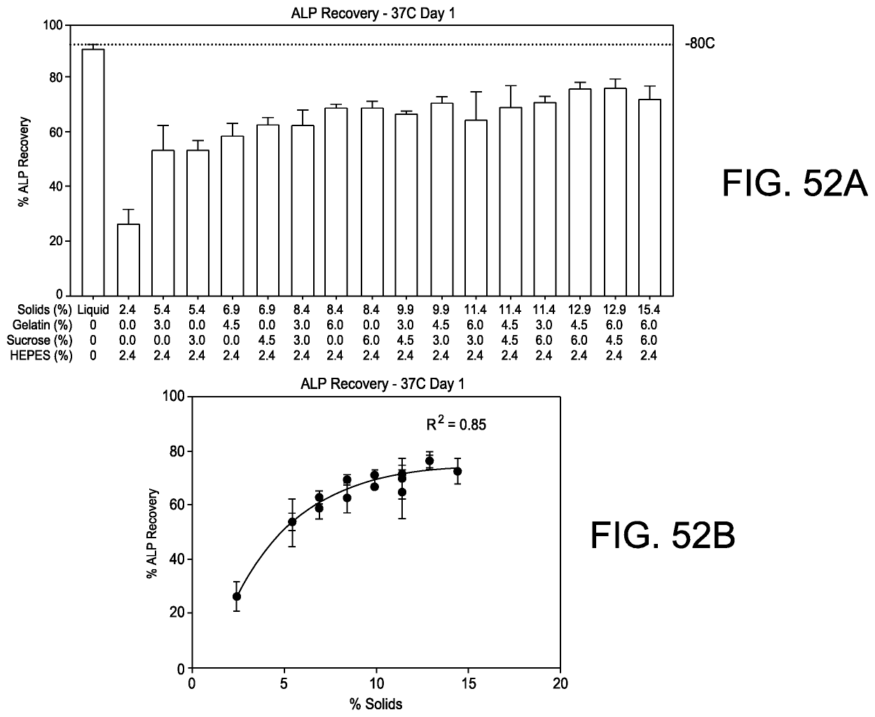

The relation between type of solid (gelatin vs. sucrose), total solid loading, and ALP recovery after storage at 37° C. was evaluated. Plasma films were analyzed after 37° C. for 1 day and compared to neat liquid plasma stored at 4° C. and −80° C. As shown in FIG. 52A, the ALP recovery is correlated with total solids loading in the formulation. Statistics from the design of experiments showed both gelatin and sucrose had equal contribution to the stability of ALP, and there was no synergistic effect. As observed in FIG. 52B, there appears to be an asymptotic trend, suggesting that increasing solids further would have a minimal increase in ALP recovery.

### Example 11. Analyte Interference Testing on Different Clinical Analyzers

### Methods:

Freshly drawn deidentified whole containing lithium heparin anticoagulant was shipped from Research Blood Components (Brighton, Mass.). Upon receipt of samples, blood samples were centrifuged at 2000 g for 10 minutes to isolate plasma. Liquid plasma was isolated, and stored at 4° C. until ready for analysis. For analysis, samples were delivered to Boston Clinical Laboratories (Waltham, Mass.) or East Side Clinical Laboratory (East Providence, R.I.), where plasma was used to reconstitute lyophilized formulations at a 1:1 volume ratio. Formulations contained either silk, sucrose, gelatin, HEPES, or a combination. Plasma was allowed to dissolve the cake for 30 seconds, before being processed on a Beckman Coulter AU680 or Cobas8000 series instrument for the entire comprehensive metabolic panel (CMP). This panel includes albumin (ALB), total protein (TP), creatinine (CREAT), blood urea nitrogen (BUN), bilirubin (BIL), calcium (CA), chloride (CL), sodium (NA), potassium (K), bicarbonate (CO2), glucose (GLUC), alkaline phosphatase (ALP), alanine transaminase (ALT), and aspartate transaminase (AST). Additional analytes, including triglycerides (TRIG) and c-reactive protein (CRP) were also run on Cobas8000 series instrumentation.

### Results:

Compatibility of the formulation of the present devices were evaluated with using two different clinical chemistry analyzers (Beckman Coulter AU680 or Cobas 8000 series) for analytes in the comprehensive metabolic panel, a routine test completed in clinical laboratories. These tests are typically completed on liquid serum or plasma. In the present example, the analyzer was used to compare readings from formulated liquid plasma to assess assay interference.

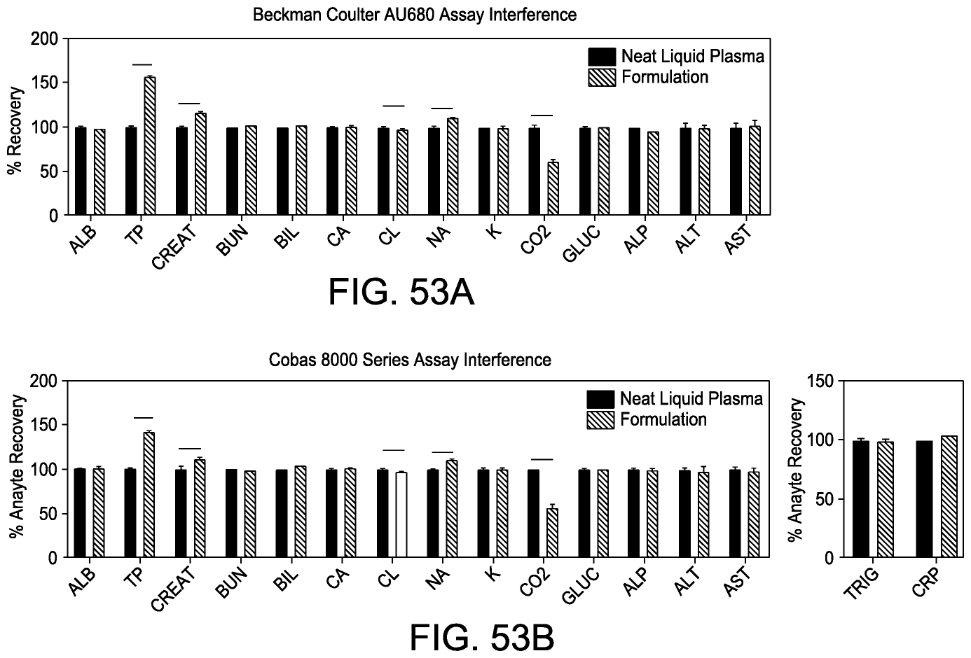

As shown in FIGS. 53A-53B, it was determined that there was no interference of formulation in the majority of analytes tested. Using CLIA guidelines (42 CFR § 493.931 2003), using a single formulation, 70% of the analytes in the CMP were within acceptable limits. Of those analytes outside of CLIA guidelines (total protein, creatinine, sodium, and bicarbonate), the mode of interference could be determined from formulation components (e.g. silk will increase total protein readings), and corrected for in later measurements. In addition, formulation was compatible with both Beckman Coulter and Roche Cobas testing platforms.

### Example 12. Analyte Process Loss after Undergoing Drying

### Methods:

Freshly drawn deidentified whole containing lithium heparin anticoagulant was shipped from Research Blood Components (Brighton, Mass.). Upon receipt of samples, blood samples were centrifuged at 2000 g for 10 minutes to isolate plasma. Silk films were fabricated by mixing plasma with lyophilized silk/sucrose/HEPES solutions at a 1:1 volume ratio. Plasma samples were cast onto 16 mm polydimethylsiloxane molds, and dried in a biosafety cabinet for up to 4 hours. Once dried, films were transferred to tubes and stored at 4° C. until ready for analysis. For analysis, samples were delivered to Boston Clinical Laboratories (Waltham, Mass.), where samples were reconstituted with deionized water to return to the neat plasma concentration. Once reconstituted, samples were run on a Beckman Coulter AU680 analyzer across the entire comprehensive metabolic panel (CMP). Samples were compared to liquid plasma samples stored at 4° C.

### Results:

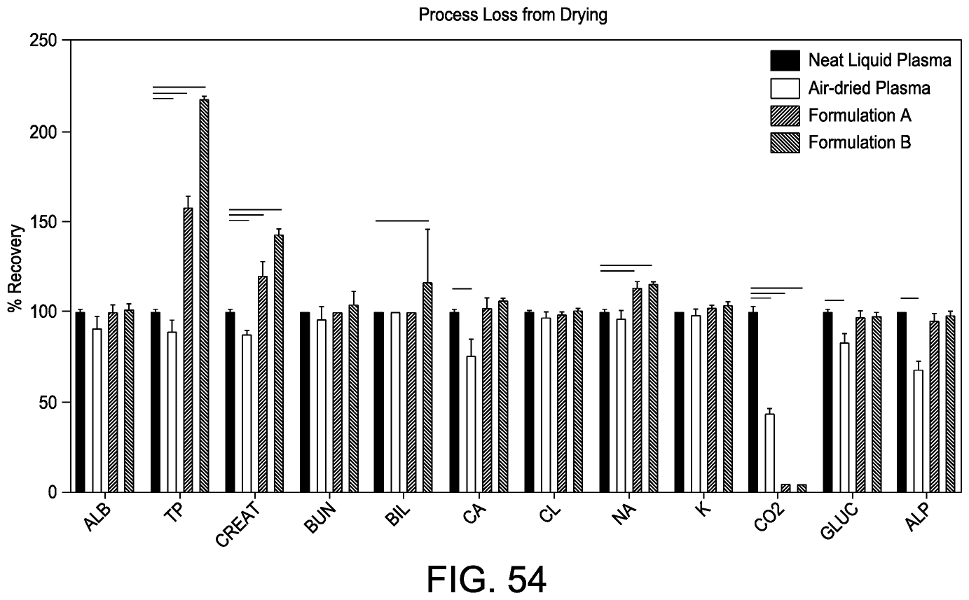

Process loss from drying was evaluated with a clinical chemistry analyzer for the analytes in the comprehensive metabolic panel (CMP). These markers, listed previously, are routine tests completed in clinical laboratories. These tests are typically completed on liquid serum or plasma. In the present example, the analyzer was used to compare readings from stored liquid plasma to air dried films to determine the process loss from drying. In addition, formulated plasma films were also tested to evaluate how formulation improves process loss. As shown in FIG. 54, recovery was analyte dependent. Analytes with known interference (total protein, creatinine, bicarbonate) continued to show similar interfering trends when dried into film format. Other analytes (e.g. BUN, chloride) exhibited minimal process loss in both film types. Several analytes in the neat film format exhibited significant process losses through the air drying, however, the physical entrapment of formulation eliminated these losses, and returned analyte levels to baseline (e.g. calcium, glucose, alkaline phosphatase).

### Example 13. 3-Day Refrigeration-Free Stability of Liver Panel Analytes

### Methods:

Freshly drawn deidentified whole containing lithium heparin anticoagulant was shipped from Research Blood Components (Brighton, Mass.). Upon receipt of samples, blood samples were centrifuged at 2000 g for 10 minutes to isolate plasma. Dried Plasma Spot (DPS) samples were prepared by pipetting 100 uL plasma onto 16 mm disk of Whatman 903 paper. Formulated films were fabricated by mixing plasma with lyophilized silk/sucrose/HEPES solutions at a 1:1 volume ratio, and then cast onto 16 mm polydimethylsiloxane molds. Both DPS and formulated films were dried in a biosafety cabinet for up to 4 hours. Once dried, samples were transferred to tubes and stored at 4° C. or 37° C. until ready for analysis. For analysis, samples were delivered to Boston Clinical Laboratories (Waltham, Mass.). DPS samples were eluted with 300 uL deionized water for 1 hour. Film samples were reconstituted with deionized water to return to the neat plasma concentration. Once ready, samples were run on a Beckman Coulter AU680 analyzer for liver panel analytes aspartate transaminase (AST) and alanine transaminase (ALT). Samples were compared to neat liquid plasma samples stored at same temperature conditions, and normalized to −80° C. stored frozen plasma samples.

### Results:

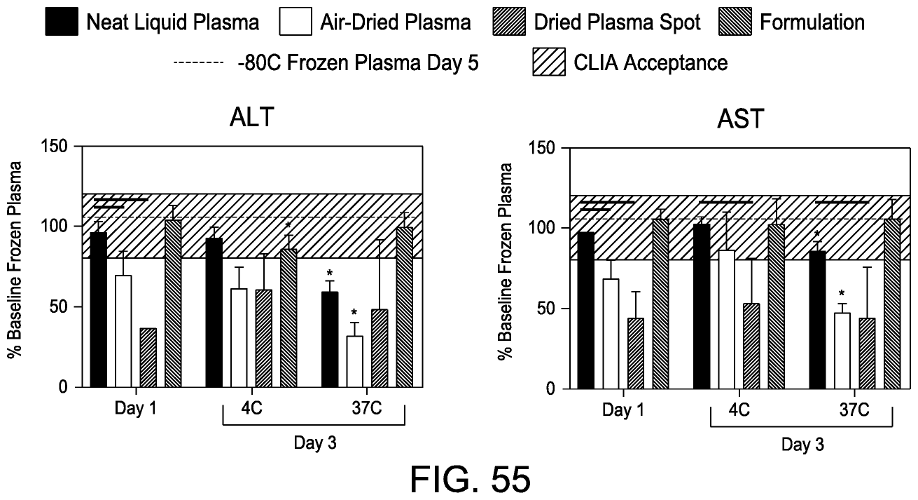

Refrigeration free stability of liver panel analytes was assessed with a clinical chemistry analyzer. These markers are routine tests completed in clinical laboratories, and typically completed using liquid serum or plasma samples. In the present example, the analyzer was used to compare readings from stored liquid plasma, plasma dried on Whatman 903 paper (dried plasma spot, DPS), and formulated air dried films to determine how formulation can improve stability of these labile enzymes. As shown in FIG. 55, the process loss from drying is overcome when dried with formulation. When stored at elevated temperatures, enzyme activity in neat liquid plasma decreases significantly after 3-days. Further, enzymes are not fully recovered and are highly variable when dried in paper (DPS), regardless of storage length or temperature. When plasma is dried with formulation, however, analyte levels remain consistent with baseline frozen plasma, and 3-day refrigeration free stability is achieved.

### Example 14. Cardiovascular Marker Thermostability

### Methods:

Freshly drawn deidentified whole containing lithium heparin anticoagulant was shipped from Research Blood Components (Brighton, Mass.). Upon receipt of samples, blood samples were centrifuged at 2000 g for 10 minutes to isolate plasma. Plasma was used to reconstitute lyophilized cakes containing gelatin/sucrose/HEPES solutions at 1:1 volume ratio. Plasma films were prepared by casting non-formulated or formulated plasma onto 16 mm polydimethylsiloxane disks and drying in a biosafety cabinet for up to 4 hours. Once dried, film samples and liquid plasma were transferred to tubes and stored at 37° C. or 45° C. for 5 days. Additional liquid plasma aliquots were stored at −80° C. for 5 days. For analysis, samples were taken out of stability and delivered to East Side Clinical Laboratory (East Providence, R.I.). Plasma film samples were reconstituted with deionized water to return to the neat plasma concentration. Once ready, samples were analyzed on Cobas8000 series instrumentation for cholesterol (CHOL), high-density lipoproteins (HDL), triglycerides (TRIG), and c-reactive protein (CRP).

### Results:

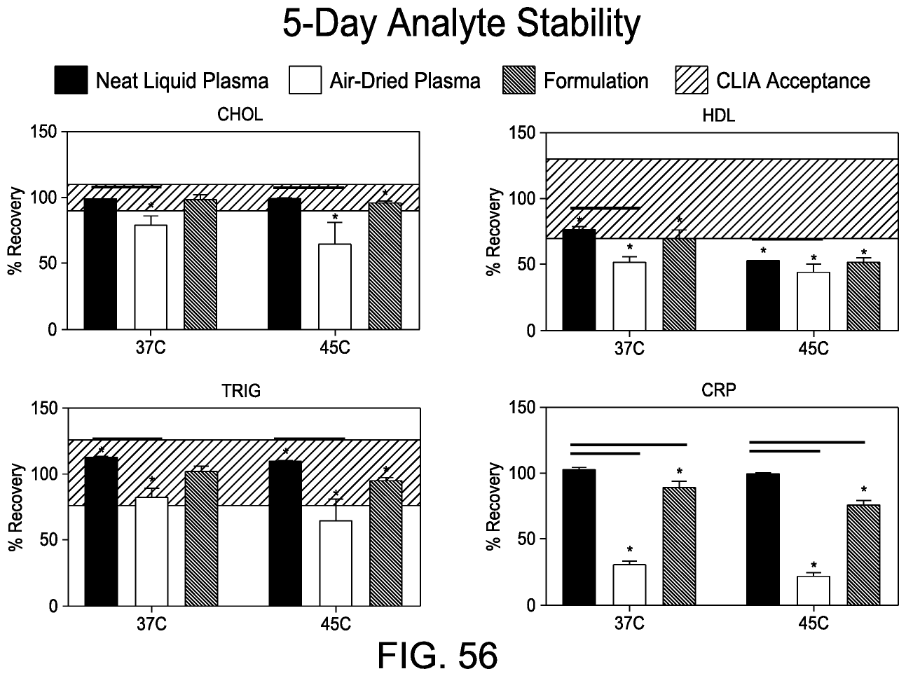

As shown in FIG. 56, recovery of analytes was analyte and temperature dependent, however in all cases, air-dried plasma (no formulation) showed a significant reduction in recovery compared to neat liquid plasma. Further, when dried with formulation, an increase in recovery against the non-formulated film was observed. Formulated films exhibited stability of CHOL and TRIG when stored at 37° C. for 5 days. Both liquid plasma and formulated films, however, showed similar significant losses in HDL recovery at elevated temperatures.

### Example 15. Formulation Effect on Plasma Separation on Different Paper Membranes

### Methods:

Freshly drawn deidentified whole containing lithium heparin anticoagulant was shipped from Research Blood Components (Brighton, Mass.). Upon receipt of samples, blood was used to reconstitute lyophilized cakes containing gelatin/sucrose/HEPES solutions at 1:1 volume ratio (individual formulation components ranged from 0-4% w/v). Formulated or neat whole blood (control) was directly pipetted onto the end of the of a LF1 (GE Healthcare), 1660, or 1667 (Ahlstrom-Munksjö) 64 mm×16 mm paper membrane. Samples were allowed to separate and fully dry overnight. ImageJ (NIH) was used to determine total area of the hematocrit and plasma region of the membranes. The plasma region of each membrane was cut and the mass of the isolated plasma region was obtained by subtracting a mass of a blank piece of each respective membrane of the same area.

### Results:

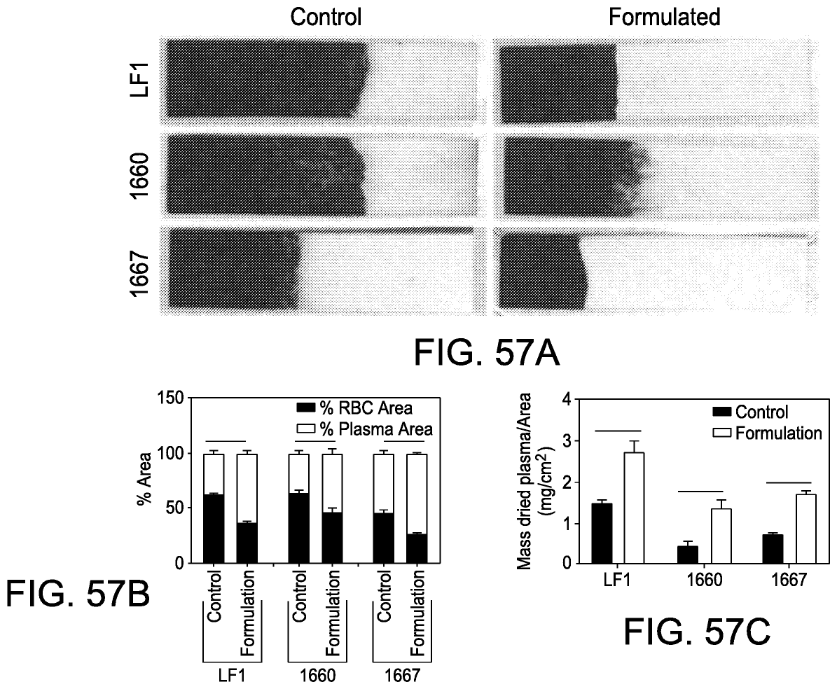

As shown in FIGS. 57A-57C, blood separates different based on the type of paper membrane and if the blood was formulated prior to blotting on the membranes. FIG. 57B, indicates that when blood was formulated, there was a significant increase in available plasma area in the paper membrane. This would ultimately allow for more tests to be completed from a single separation membrane due to the increase punch-able area. In addition, the increase in mass in the plasma region per square area suggests that formulation is drying with the plasma, thereby protecting the analytes during drying and storage.

### Example 16. Formulation Component Effect on Blood Separation on Paper

### Methods:

Freshly drawn deidentified whole containing lithium heparin anticoagulant was shipped from Research Blood Components (Brighton, Mass.). Upon receipt of samples, blood was used to reconstitute lyophilized cakes containing silk, sucrose, HEPES, gelatin, and/or bovine serum albumin (BSA) at a 1:1 volume ratio (individual formulation components ranged from 0-4% w/v). Formulated or neat whole blood was directly pipetted onto the end of the of a LF1 (GE Healthcare) 64 mm×16 mm paper membrane. Samples were allowed to separate and fully dry overnight. ImageJ (NIH) was used to determine total area of the hematocrit and plasma region of the membranes. The plasma region of each membrane was cut and the mass of the isolated plasma region was obtained by subtracting a mass of a blank piece of each respective membrane of the same area.

### Results:

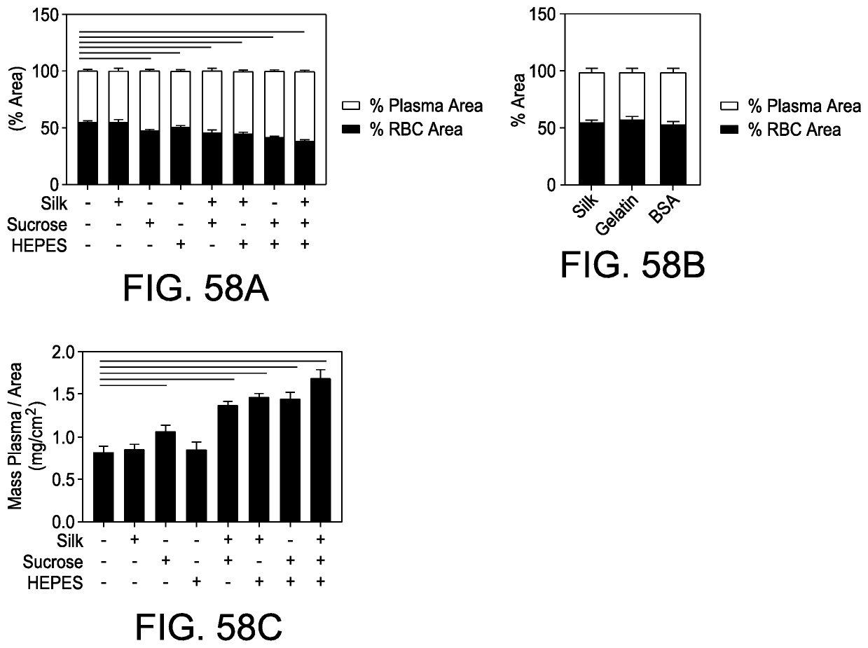

As shown in FIG. 58A, inclusion of different formulation components has a different effect on the plasma separation behavior. For example, indicates silk and HEPES alone do not significantly increase the available plasma area in the membrane compared to the neat/control sample. When combined, however, the available plasma significantly increases, suggesting a synergistic effect is occurring. Furthermore, sucrose alone in formulation had a greater effect of available plasma area than silk, even though the same loading amount was used between the two components. There was no difference in the separation behavior of the membranes when different proteins were evaluated (FIG. 58B). A similar effect of available plasma area and mass of dried plasma in the isolate plasma fraction can be observed in FIG. 58C, overall suggesting formulation is drying within the plasma region in select formulations.

### Example 17. Formulation Spreading Along Plasma Separation Membranes

### Methods:

Deionized water was used to reconstitute lyophilized cakes containing silk, sucrose, and/or HEPES at a 1:1 volume ratio. Formulation was directly pipetted onto the end of the of a LF1 (GE Healthcare) 64 mm×16 mm paper membrane. Samples were allowed to spread and fully dry overnight. Once dry, a 5 mm punch was used to cut segments along the strip. Mass of formulation within the punch were determined by subtracting the mass of non-formulated punches.

### Results:

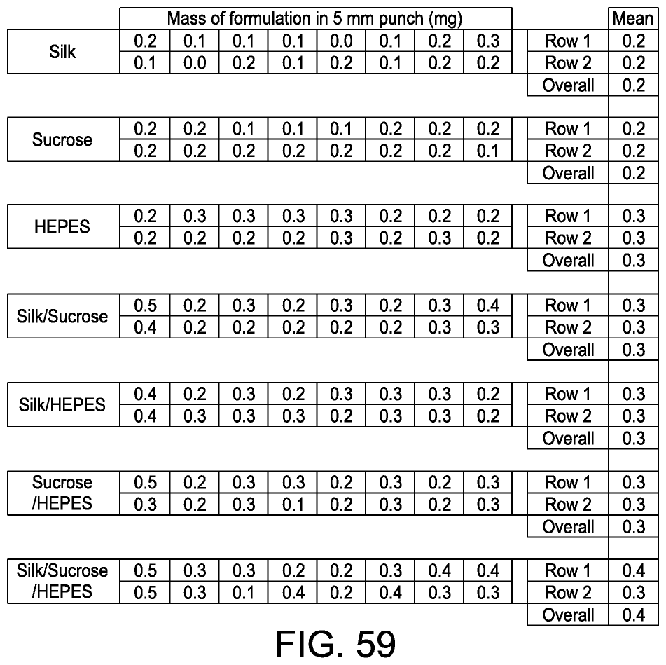

As shown in FIG. 59, formulation is allowed to fully spread throughout the entire strip. The increase in mass throughout the entire strip corroborates data with increased mass in plasma when formulated blood in deposited onto the membrane. This suggests that formulated blood will separate and dry with formulation incorporated within both the red blood cell and plasma region of the paper membrane.

### Example 18. Liver Enzyme Linearity

### Methods:

Freshly drawn deidentified whole blood containing lithium heparin anticoagulant was shipped from Research Blood Components (Brighton, Mass.). Upon receipt, an aliquot of the blood samples were centrifuged at 2000 g for 10 minutes to isolate plasma. Plasma was used to reconstitute lyophilized cakes containing silk/sucrose/HEPES solutions at 1:1 volume ratio. Plasma films were prepared by casting non-formulated or formulated plasma onto 16 mm polydimethylsiloxane disks and drying in a biosafety cabinet for up to 4 hours. For AdvanceDX 100 (ADx) samples, 200 uL whole blood was directly pipetted onto the end of the card, and allowed to separate and dry. Once dried, film, ADx, and liquid plasma samples were transferred to tubes and stored at 4° C. or 37° C. for 3 days. For analysis, samples were taken out of stability and analyzed using a Beckman Coulter AU680 (Boston Clinical Laboratories, Waltham, Mass.), Roche cobas 8000 series (East Side Clinical Laboratory, East Providence, R.I.), or a Roche cobas c111 (Vaxess Technologies, Cambridge, Mass.). Plasma film samples were reconstituted with deionized water to return to the neat plasma concentration. Plasma regions of the ADx samples were punched and eluted in deionized water at room temperature for 1 hour while vortexing. Once ready, samples were assayed for aspartate aminotransferase (AST), alanine aminotransferase (ALT), and sodium. Plasma sodium was used to determine the dilution factor for the ADx samples.

### Results:

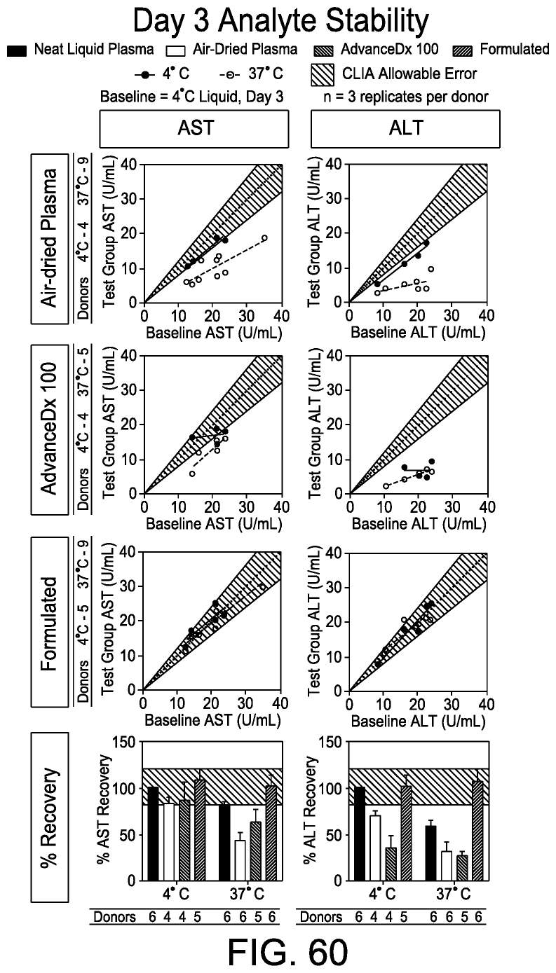

As shown in FIG. 60, ALT and AST were more stable in formulated films than in all other formats at 3-day ground shipment temperature of 37° C. compared to baseline. Analytes recovered from plasma films and AdvanceDX100 cards were in some instances correlated to liquid plasma analyte levels upon initial recovery; however, at elevated storage temperatures the original correction factor was no longer viable. Formulated films, however, exhibited similar recoveries regardless of storage conditions.

### EQUIVALENTS

While this invention has been particularly shown and described with references to preferred embodiments thereof, it will be understood by those skilled in the art that various changes in form and details may be made therein without departing from the spirit and scope of the invention as defined by the appended claims. Those skilled in the art will recognize, or be able to ascertain using no more than routine experimentation, many equivalents to the specific embodiments of the invention described specifically herein. Such equivalents are intended to be encompassed in the scope of the appended claims.

1. A biological sample collection and processing device comprising:
(i) a sample collection and mixing portion configured to receive and mix a biological sample, the sample collection and mixing portion including a silk fibroin composition, the sample collection and mixing portion being configured to allow a sufficient amount of the biological sample and the silk fibroin composition to form a mixture; and
(ii) a drying and recovery portion configured to allow substantially drying the biological sample and/or the mixture.

2. The device of claim 1, wherein:
(a) the silk fibroin composition is substantially dried, lyophilized, air-dried, spray dried, or liquid;
(b) the silk fibroin composition comprises about 0.1% w/v to about 20% w/v silk fibroin;
(c) the silk fibroin composition comprises a population of silk fibroin having an average molecular weight of about 1 kDa to about 250 kDa;
(d) the silk fibroin composition comprises a 10 minute boil (10 MB) silk fibroin composition;
(e) the silk fibroin composition comprises a 30 minute boil (30 MB) silk fibroin composition;
(f) the silk fibroin composition comprises a 60 minute boil (60 MB) silk fibroin composition;
(g) the silk fibroin composition comprises a 120 minute boil (120 MB) silk fibroin composition;
(h) the silk fibroin composition comprises a 180 minute boil (180 MB) silk fibroin composition;
(i) the silk fibroin composition comprises a 240 minute boil (240 MB) silk fibroin composition;
(j) the silk fibroin composition comprises a 300 minute boil (300 MB) silk fibroin composition;
(k) the silk fibroin composition comprises a 360 minute boil (360 MB) silk fibroin composition;
(l) the silk fibroin composition comprises a 420 minute boil (420 MB) silk fibroin composition;
(m) the silk fibroin composition comprises a 480 minute boil (480 MB) silk fibroin composition; and/or
(n) the silk fibroin composition comprises a population of silk fibroin having an average polydispersity index (PDI) of about 5 to about 20.

3. The device of claim 1, wherein the device comprises about 0.01 mg to about 100 mg of the silk fibroin composition or about 25 μL to about 300 μL of the silk fibroin composition.

4.-6. (canceled)

7. The device of claim 1, wherein the silk fibroin composition further comprises an excipient.

8. The device of claim 7, wherein:
(a) the excipient is a sugar and/or a sugar alcohol chosen from sucrose, trehalose, maltose, sorbitol, mannitol, glycerol, or a combination thereof;
(b) the excipient is a protein chosen from a gelatin protein, an albumin protein, an elastin protein, a collagen protein, a cellulose protein, or a combination thereof;
(c) the excipient is a polymer chosen from a polyethylene glycol (PEG), a polyethylene oxide (PEO), a hyaluronic acid, a carboxymethylcellulose (CMC), a polyvinylpyrrolidone (PVP), a polyvinyl alcohol (PVA), or a combination thereof;
(d) the excipient is a divalent cation chosen from Ca²⁺, Mg²⁺, Mn²⁺, Cu²⁺ or a combination thereof;
(e) the excipient is a salt comprising monosodium glutamate;
(f) the excipient is a buffer chosen from a Tris buffer, a Tris EDTA buffer, a citrate buffer, a phosphate buffer, a MOPS buffer, a HEPES buffer, or a combination thereof;
(g) the excipient is an amino acid chosen from L-glutamic acid, lysine, or a combination thereof; and/or
(h) the excipient is a solvent chosen from DMSO, methanol, ethanol, or a combination thereof.

9. The device of claim 1, wherein:
(a) the device is configured to reconstitute a dried silk fibroin composition to a pre-defined volume; and/or
(b) the device is configured to meter about 10 μL to about 300 μL of the biological sample.

10. (canceled)

11. The device of claim 1, wherein the sample collection and mixing portion includes a tube which is a capillary tube and/or a removable absorbent cap configured to absorb excess sample.

12. (canceled)

13. The device of claim 1, wherein the drying and recovery portion is a removable container configured to be releasably secured to the sample collection and mixing portion.

14. The device of claim 1 further comprising:
(a) a porous membrane for plasma separation positioned in the collection and mixing portion such that it functions as a bottom of the collection portion; and/or
(b) a sample collector positioned in the collection and mixing portion, wherein the sample collector is coated with anticoagulant and/or comprises a finger sample wiper feature.

15.-17. (canceled)

18. The device of claim 1, wherein:
(a) the drying and recovery portion includes a removable desiccant cap configured to include desiccant material; and/or
(b) the drying and recovery portion includes a tube and a well positioned in the tube, the well being configured to contain the biological sample, the silk fibroin composition, and/or the mixture for easy elution.

19. (canceled)

20. The device of claim 19, further comprising a plunger configured to create a negative pressure in the well thereby drawing the sample through a porous membrane and into the well directly or via a part that channels the flow, wherein the tube contains a desiccant configured to facilitate air-drying of the biological sample and/or the mixture in the well, and wherein the desiccant is chosen from the group comprising a molecular sieve, a silica gel, a montmorillonite clay, a calcium oxide, a calcium sulfate, and mixtures thereof.

21.-22. (canceled)

23. The device of claim 19, wherein:
(a) the well includes a geometry to spread the collected sample over a large surface area;
(b) the well includes a centrally located dimple to provide adequate sample depth for pipette access and ribs to guide a tip of a pipette to the dimple; and/or
(c) the well includes posts to create a meniscus in the sample to facilitate air-drying.

24.-25. (canceled)

26. A sample collection device comprising:
a tube;
a well positioned in the tube, the well being configured to receive and dry a mixture of a biological sample and a stabilizing composition comprising a polymer, a sugar, and/or a sugar alcohol; and
a sample collection assembly configured to receive and mix the biological sample with the stabilizing composition, and to deliver the mixture to the well, the sample collection assembly being configured to allow a precise amount of the biological sample and the stabilizing composition to form the mixture.

27.-55. (canceled)

56. The device of claim 1, wherein the drying occurs:
(a) occurs within about 48 hours;
(b) within about 1 to about 10 hours;
(c) within about 3 to about 6 hours;
(d) within about 1 to about 2 hours; or
(e) within less than about 1 hours.

57. The device of claim 1, wherein the device is configured to produce and to retain a substantially dried composition, wherein the substantially dried composition stabilizes the structure, integrity, configuration, function, and/or activity of a biological sample, or at least one fraction, component, and/or analyte thereof for a period of time.

58.-62. (canceled)

63. The device of claim 1, wherein the biological sample is from a human subject.

64.-80. (canceled)

81. A method of making a substantially dried composition or identifying and/or measuring the level of at least one fraction, component, and/or analyte of a biological sample, said method comprising the steps of:
(i) metering a pre-determined amount of a biological sample into a device of claim 1, wherein the device comprises a silk fibroin composition;
(ii) mixing the biological sample of (i) with the lyophilized silk fibroin composition of (i) to form a mixture;
(iii) drying the biological sample and/or the mixture,
thereby making a substantially dried composition.

82.-86. (canceled)

87. A substantially dried composition made by the use of a device of claim 1.

88.-97. (canceled)

98. A mixture and/or an analysis sample made by the use of a device of claim 1.

99. A reaction mixture and/or an analysis sample made by reconstituting the substantially dried composition of claim 87.

100.-103. (canceled)
